SEQUENCE LISTING (24 sequences)

SEQ ID NO: 1 - DNA, 20 bp, synthetic construct
  source 1..20: mol_type = other DNA
  misc_feature 1..20: note = PyroMark assay primers for measuring mt.11944 heteroplasmy - forward
cttcattattagcctcttac

SEQ ID NO: 2 - DNA, 20 bp, synthetic construct
  source 1..20: mol_type = other DNA
  misc_feature 1..20: note = PyroMark assay primers for measuring mt.11944 heteroplasmy - reverse
gtctgagtgtatatatcatg

SEQ ID NO: 3 - DNA, 20 bp, synthetic construct
  source 1..20: mol_type = other DNA
  misc_feature 1..20: note = PyroMark assay primers for measuring mt.11944 heteroplasmy - sequencing
ctattgaatttatggtgact

SEQ ID NO: 4 - DNA, 19 bp, synthetic construct
  source 1..19: mol_type = other DNA
  misc_feature 1..19: note = PyroMark assay primers for measuring mt.11944 heteroplasmy - forward
atattctccaacaacaacg

SEQ ID NO: 5 - DNA, 19 bp, synthetic construct
  source 1..19: mol_type = other DNA
  misc_feature 1..19: note = PyroMark assay primers for measuring mt.11944 heteroplasmy - reverse
gttattattagtcgtgagg

SEQ ID NO: 6 - DNA, 17 bp, synthetic construct
  source 1..17: mol_type = other DNA
  misc_feature 1..17: note = PyroMark assay primers for measuring mt.11944 heteroplasmy - sequencing
ctattgctgatggtagt

SEQ ID NO: 7 - DNA, 23 bp, synthetic construct
  source 1..23: mol_type = other DNA
  misc_feature 1..23: note = ddPCR EvaGreen primers ND5 - forward
tgcctagtaatcggaagcctcgc

SEQ ID NO: 8 - DNA, 20 bp, synthetic construct
  source 1..20: mol_type = other DNA
  misc_feature 1..20: note = ddPCR EvaGreen primers ND5 - reverse
tcaggcgttggtgttgcagg

SEQ ID NO: 9 - DNA, 20 bp, synthetic construct
  source 1..20: mol_type = other DNA
  misc_feature 1..20: note = ddPCR EvaGreen primers VDAC1 - forward
ctcccacatacgccgatctt

SEQ ID NO: 10 - DNA, 19 bp, synthetic construct
  source 1..19: mol_type = other DNA
  misc_feature 1..19: note = ddPCR EvaGreen primers VDAC1 - reverse
gccgtagcccttggtgaag

SEQ ID NO: 11 - DNA, 45 bp, synthetic construct
  source 1..45: mol_type = other DNA
  misc_feature 1..45: note = Mt-Nd5 m. 12,436
tttggcctccacccatgactaccatcagcaatagaaggccctaca

SEQ ID NO: 12 - DNA, 45 bp, synthetic construct
  source 1..45: mol_type = other DNA
  misc_feature 1..45: note = Mt-Nd5 m. 11,944
taattacaacctggcactgagtcaccataaattcaatagaaaact

SEQ ID NO: 13 - DNA, 19 bp, synthetic construct
  source 1..19: mol_type = other DNA
  misc_feature 1..19: note = Reverse PCR primer for mt.12,436
  misc_feature 1: note = 5' biotin
gttattattagtcgtgagg

SEQ ID NO: 14 - DNA, 20 bp, synthetic construct
  source 1..20: mol_type = other DNA
  misc_feature 1..20: note = Reverse PCR primer for mt.11,944
  misc_feature 1: note = 5' Biotin
gtctgagtgtatatatcatg

SEQ ID NO: 15 - DNA, 15 bp, synthetic construct
  source 1..15: mol_type = other DNA
  misc_feature 1..15: note = Sequencing primer for mt.12,436
ttggcctccacccat

SEQ ID NO: 16 - DNA, 18 bp, synthetic construct
  source 1..18: mol_type = other DNA
  misc_feature 1..18: note = Sequencing primer for mt.11,944
taattacaacctggcact

SEQ ID NO: 17 - DNA, 28 bp, synthetic construct
  source 1..28: mol_type = other DNA
  misc_feature 1..28: note = Forward for LbNOX
ggtggtgctagccgcatgaaggtcaccg

SEQ ID NO: 18 - DNA, 29 bp, synthetic construct
  source 1..29: mol_type = other DNA
  misc_feature 1..29: note = Forward for mitoLbNOX
ggtggtgctagccgcatgctcgctacaag

SEQ ID NO: 19 - DNA, 30 bp, synthetic construct
  source 1..30: mol_type = other DNA
  misc_feature 1..30: note = Reverse
ggtggtggatccttacttgtcatcgtcatc

SEQ ID NO: 20 - DNA, 24 bp, synthetic construct
  source 1..24: mol_type = other DNA
  misc_feature 1..24: note = Forward primer 1
actgatattactatccctaggagg

SEQ ID NO: 21 - DNA, 21 bp, synthetic construct
  source 1..21: mol_type = other DNA
  misc_feature 1..21: note = Reverse primer 1
tttgagtagaaccctgttagg

SEQ ID NO: 22 - DNA, 19 bp, synthetic construct
  source 1..19: mol_type = other DNA
  misc_feature 1..19: note = Forward primer 2
ggcctgataatagtgacgc

SEQ ID NO: 23 - DNA, 23 bp, synthetic construct
  source 1..23: mol_type = other DNA
  misc_feature 1..23: note = Reverse primer 2
ggttgggtttagtttttgtttgg

SEQ ID NO: 24 - protein, 4 aa, synthetic construct
  REGION 1..4: note = Figure 40H y axis
GSSG

(19) United States
(12) Patent Application Publication
(10) Pub. No.: US 2026/0263567 A1
(43) Pub. Date: Sep. 10, 2026

(54) TUMOUR SENSITISATION TO CHECKPOINT INHIBITORS WITH REDOX STATUS MODIFIER

(71) Applicant: CANCER RESEARCH TECHNOLOGY LIMITED, London (GB)

(72) Inventors: Payam GAMMAGE, London (GB); Mahnoor MAHMOOD, New York, NY (US)

(21) Appl. No.: 19/123,256

(22) PCT Filed: Oct. 24, 2023
(86) PCT No.: PCT/GB2023/052787

§ 371 Date: Apr. 22, 2025

(30) Foreign Application Priority Data
Oct. 24, 2022 (GB) 2215725.9
Mar. 21, 2023 (GB) 2304109.8

(51) Int. Cl. A61K 38/44; A61K 39/00; A61K 39/395; A61P 35/00

(52) U.S. Cl. CPC A61K 38/44; A61K 39/3955; A61P 35/00; A61K 2039/505

(57) ABSTRACT

The present invention relates to methods of sensitising a subject having a cancer or a pre-cancer to treatment with an immune checkpoint inhibitor, as well as agents for use in sensitising a subject to such treatment.

The present invention relates to methods of sensitising a subject having a cancer or a pre-cancer to treatment with an immune checkpoint inhibitor, as well as agents for use in sensitising a subject to such treatment.

### BACKGROUND

Cancer immunotherapy involves the attack of cancer cells by a patient's immune system. Regulation and activation of T lymphocytes depends on signaling by the T-cell receptor and co-signaling receptors that deliver positive or negative signals for activation. Immune responses by T cells are controlled by a balance of costimulatory and inhibitory signals, called immune checkpoints.

Immunotherapy with immune checkpoint inhibitors is revolutionising cancer therapy. However, some patients show little or no improvement with immune checkpoint inhibitor therapies. Accordingly, methods for sensitising patients to such treatments are still needed. The present invention aims to address this need at least in part.

### BRIEF SUMMARY OF THE DISCLOSURE

The present invention is based on the inventors' surprising finding that cancer cells with heteroplasmic deleterious mutations in mitochondrial DNA (mtDNA) may have an altered tumour microenvironment. Specifically, the present inventors have shown that cancer cells with a deleterious mtDNA mutation present at a high mutation load are associated with different immune cell populations being present in the tumour microenvironment. As discussed in the Examples section of the present application, the inventors have found that microenvironments of tumours comprising such cancer cells are enriched in Natural Killer (NK) cells, monocytes, CD4+ T cells, and interferon-stimulated gene (ISG) expressing immune cells, but have reduced macrophage levels and tumour associated neutrophil levels, as compared to the microenvironments of tumours with cancer cells that have no or low deleterious mtDNA mutation load.

The inventors believe that the presence of a deleterious mtDNA mutation load alters the cancer cell metabolism in a way that alters the tumour microenvironment making it conducive to infiltration by certain populations of immune cells. As discussed in the Examples section of the present disclosure, the inventors have identified that cancer cells with heteroplasmic mutations in the MT-ND5 gene showed increased levels of reduced nicotinamide adenine dinucleotide (NADH) leading to disrupted NAD+:NADH ratio and altered cellular redox balance. This may result in reverse flux of Malate Dehydrogenase 2 (MDH2) and accumulation of cytosolically derived malate via Malate Dehydrogenase 1 (MDH1). The increased MDH1 activity may drive glycolysis and result in excess glucose consumption and excess lactate release. Indeed, the inventors have shown that these mutations promoted utilization of pyruvate as a terminal electron acceptor and increased glycolytic flux driven by an over-reduced NAD pool and NADH shuttling between GAPDH and MDH1, mediating a Warburg-like metabolic shift. Surprisingly, the inventors found that, despite these changes, oxygen consumption and ATP synthesis remained unaffected at a 60% mutation load (also referred to herein as “variant allele frequency” or “VAF”), although the inventors believe that these parameters would be impacted by a higher mutation load. Without wishing to being bound by this hypothesis, the inventors believe that these metabolic changes promote the recruitment and/or survival of specific immune cell types (such as those mentioned above) into the tumour, which are less sensitive to altered redox status (for example an altered glucose to lactate ratio). This reduced sensitivity, the inventors hypothesise, could be due to the cells' preferential utilisation of lactate as a carbon fuel source or lower dependency on glucose.

The inventors then embarked on a study to determine whether these findings might be associated with clinical outcome. Using a mouse model, the inventors surprisingly showed that tumours with >40% VAF responded well to a PD1 inhibitor, whereas tumours with little or no VAF responded less favourably.

The inventors found that this difference in treatment responsiveness was valid in the context of treatment with an immune checkpoint inhibitor (such as a PD-1 inhibitor, PD-L1 inhibitor, or CTLA4 inhibitor). These findings were also supported by a retrospective study on a small clinical cohort of human patients with >50% VAF due to mutations in a variety of different mtDNA genes (such as MT-COI, MT-ND4, MT-CYB, MT-TY, and/or the mtDNA control region). The inventors believe that this difference in responsiveness to treatment with an immune checkpoint inhibitor (e.g. PD-1 inhibitor, a PD-L1 inhibitor and/or a CTLA4 inhibitor) is due to the metabolic changes caused by high VAF sensitising the cancer or pre-cancer cells in the tumour to the treatment (and the resultant changes to the immune microenvironment of the cancer or pre-cancer). Based on these data, the inventors conclude that a cancer or pre-cancer can be sensitised to treatment with an immune checkpoint inhibitor (such as PD-1 inhibitor, a PD-L1 inhibitor, and/or CTLA4 inhibitor) by mimicking this metabolic change (i.e. by altering the redox status in the cancer or a pre-cancer, for example the lactate to glucose ratio in the cancer or a pre-cancer).

The inventors further showed that upon providing to a cancer cell (e.g. a melanoma cancer cell) an agent that alters the redox status, for example lactate to glucose ratio, the cancer cells had an increased response to immune checkpoint inhibitor treatment (such as anti-PD1 treatment). Specifically, the inventors modified wild-type Hcmel12 cells to constitutively express cytoLBnox, which reproduces key elements of the cell-extrinsic, mutant Mt-Nd5-associated metabolic phenotype, notably glucose uptake and lactate release. When grafted into mice, Hcmel12 cytoLBnox tumours demonstrated comparable time to endpoint and tumour weight at endpoint as wild-type or Mt-Nd5 mutant tumours. However, when challenged with anti-PD1 treatment, Hcmel12 cytoLBnox tumours recapitulate the response of Hcmel12 mt-Nd5 m.12,436^{80%} tumours, confirming that specific changes in redox metabolism are sufficient to sensitize the tumour to immune checkpoint blockade (for example a PD-1 inhibitor, a PD-L1 inhibitor, and/or CTLA4 inhibitor).

Moreover, the inventors have found that treatment responsiveness to an immune checkpoint inhibitor may be further (synergistically) improved in tumours with a high mtDNA mutation load or expressing cytoLbNOX, by co-treatment with compounds that reduce levels of tumour resident neutrophils (such as anti-Ly6G antibodies).

The present inventors have also shown that agents that alter the redox status, for example the lactate to glucose ratio, in a cancer or a pre-cancer (such as cytoLbNOX or mitoLbNOX) may increase the sensitivity to an immune checkpoint inhibitor in a cancer that has baseline sensitivity to immune checkpoint inhibitors, as shown in the immunogenic 4434 mouse model.

The invention therefore provides an agent that alters the redox status (for example alters the lactate to glucose ratio) in a cancer or a pre-cancer for use in sensitising a subject having cancer or pre-cancer to an immune checkpoint inhibitor.

Also provided is an immune checkpoint inhibitor for use in treating a subject having a cancer or a pre-cancer, wherein the subject has been exposed to an agent that alters the redox status (for example alters the lactate to glucose ratio) in the cancer or pre-cancer.

The invention also provides a method of sensitising a subject having a cancer or a pre-cancer to an immune checkpoint inhibitor, comprising exposing the subject to an agent that alters the redox status (for example alters the lactate to glucose ratio) in the cancer or pre-cancer.

Further provided is a method of treating a cancer or a pre-cancer in a subject, comprising administering an immune checkpoint inhibitor to the subject, wherein the subject has been exposed to an agent that alters the redox status (for example alters the lactate to glucose ratio) in the cancer or pre-cancer.

The invention also provides a method of treating a cancer or a pre-cancer in a subject, comprising:
(i) exposing the subject to an agent that alters the redox status (for example alters the lactate to glucose ratio) in the cancer or pre-cancer; and
(ii) administering an immune checkpoint inhibitor to the subject.

Suitably, the immune checkpoint inhibitor may be selected from the group consisting of a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and KIR inhibitor.

Suitably, the immune checkpoint inhibitor may be selected from the group consisting of a PD-1 inhibitor, a PD-L1 inhibitor, and CTLA4 inhibitor.

Suitably, the agent may increase the lactate to glucose ratio.

Suitably, the agent alters (e.g. increases) the lactate to glucose ratio in the interstitial fluid of the cancer or pre-cancer.

Suitably, the increase in lactate to glucose ratio may be to above 3:1.

Suitably, a sample of the cancer or precancer may have a deleterious mitochondrial DNA (mtDNA) mutation load of less than 50%.

Suitably, the deleterious mitochondrial DNA (mtDNA) mutation load may be less than 40%, less than 30%, or less than 20%.

Suitably, the agent may be selected from the group consisting of:
a) a compound that drives glycolytic flux through MDH1, optionally wherein the compound is selected from the group consisting of: isocitrate, aconitate, citrate, oxaloacetate, NADH and NAD+ precursors;
b) a compound that modulates NAD(H) redox handling via the malate-aspartate shuttle, optionally wherein the compound is selected from the group consisting of: isocitrate, aconitate, citrate, oxaloacetate, malate, fumarate, argininosuccinate
c) lactate;
d) a glucose metabolising enzyme and/or a lactate metabolising enzyme;
e) an inhibitor of an enzyme that decreases glycolytic flux in cancer cells or pre-cancer cells, optionally wherein the enzyme is pyruvate dehydrogenase or pyruvate carboxylase, optionally wherein the inhibitor is a small molecule;
f) an activator of an enzyme that increases gycolytic flux in cancer cells or pre-cancer cells;
g) an activator of an enzyme that increases lactate efflux in cancer cells or pre-cancer cells, optionally wherein the enzyme is MDH1 or GAPDH, optionally wherein the activator is a small molecule;
h) an inhibitor of an enzyme that decreases lactate efflux in cancer cells or pre-cancer cells;
i) a small molecule inhibitor of an enzyme in the malate-aspartate shuttle, optionally wherein the enzyme is selected from the group consisting of GOT1, GOT2, MDH1, MDH2, Glutamate-Aspartate carrier, and a-ketoglutarate-malate carrier;
j) a small molecule activator of an enzyme in the malate-aspartate shuttle, optionally wherein the enzyme is selected from the group consisting of GOT1, GOT2, MDH1, MDH2, Glutamate-Aspartate carrier, and a-ketoglutarate-malate carrier;
k) an inhibitor of complex I, complex II, complex III or complex IV;
l) a compound that increases a deleterious mtDNA mutation load in the cancer or pre-cancer, optionally wherein the compound induces a deleterious mtDNA mutation; and/or
m) a compound that decreases neutrophils in the subject, and/or decreases neutrophils in the cancer or pre-cancer, optionally wherein the neutrophils are tumour infiltrating neutrophils (TANs).

Suitably, the agent may be selected from the group consisting of a NADH oxidase and a NADPH oxidase.

Suitably the agent may be the enzyme NADH oxidase (for example from Lactobacillus brevis) or a nucleic acid that encodes said enzyme. Suitably, the NADH oxidase may be cytosolic or mitochondrial.

Suitably, the agent may be for use in combination with a tumour-associated neutrophil 20 reducing compound (such as anti-Ly6G antibody).

Suitably, the cancer or pre-cancer may be selected from the group consisting of: a childhood cancer, haematological cancer, and a myeloid cancer.

Suitably, the childhood cancer may be selected from the group consisting of: leukemia, brain cancer, spinal cord cancer, neuroblastoma, Wilms tumor, lymphoma (such as Hodgkin and non-Hodgkin), rhabdomyosarcoma, retinoblastoma, and bone cancer (such as osteosarcoma and Ewing sarcoma).

Suitably, the PD-1 inhibitor may be nivolumab.

Suitably, the compound that increases a deleterious mtDNA mutation load in the cancer or pre-cancer may be selected from the group consisting of a mitochondrial base editing enzyme (such as DdCBEs) and a mitochondrial heteroplasmy manipulating enzyme (such as mtZFNs or mitoTALENs).

Suitably, the deleterious mtDNA mutation may be selected from the group consisting of:
(i) a tRNA mutation having a MitoTIP RAW score of at least 12.6, or at least 16.25;
(ii) a rRNA mutation;
(iii) a truncation mutation in a mtDNA gene;
(iv) a missense mutation in a mtDNA gene, wherein the missense mutation has an Apogee score of more than 0.5, optionally wherein the missense mutation is selected from a frameshift mutation, an insertion mutation or a deletion mutation; and/or
(v) a mutation in a mtDNA D-loop region selected from the group consisting of: the H-strand promoter (545-567), MT-HV2 (hypervariable segment 2) m.57-372, and MT-HV1 (hypervariable segment 1)—m.16024-16390.

Suitably, the deleterious mtDNA mutation may be in a gene selected from the group consisting of: MT-ND5, MT-ND1, MT-ND2, MT-ND3, MT-ND4, MT-ND4L, MT-ND6, MT-CO1, MT-C02, MT-C03, MT-CYB, MT-ATP6, MT-ATP8, MT-TL1, MT-TA, MT-TC, MT-TD, MT-TE, MT-TF, MT-TG, MT-TH, MT-TI, MT-TK, MT-TL2, MT-TM, MT-TN, MT-TP, MT-TQ, MT-TR, MT-TS1, MT-TS2, MT-TT, MT-TV, MT-TW, MT-TY, MT-RNR1 and MT-RNR2.

Suitably, the MT-ND5 deleterious mtDNA mutation may be a truncating mutation that is in a region selected from: m.12418-12425:A indel or m.12385-12390:C indel.

Suitably, the deleterious mtDNA mutation may be a truncation, missense, insertion, or frameshift mutation.

As would be clear to a person of skill in the art, any embodiments that relate to sensitising a subject to an immune checkpoint inhibitor (such as PD-1 inhibitor, PD-L1 inhibitor, and/or CTLA4 inhibitor) (including methods or agents for use in sensitising) equally apply to the methods of treatment (or agents for use in treatment) described herein unless the context specifically requires otherwise.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows that mtDNA mutations are abundant in different cancers and provides information on MT-ND5. A—Percentage of well-covered tumours with different types of somatic mtDNA variants per cancer type. Boxes from left to right are: truncating, 2+ types non truncating, rRNA, tRNA, missense, silent, wildtype. Right: number of well-covered samples per cancer type. NSC, non-small-cell cancer. Data from Gorelick et al., 2021. B—Circular mtDNA genome annotated with 73 homopolymer repeat loci ≥5 bp in length. Dot height from the circular mtDNA genome indicates the number of affected samples and dot width indicates the length of the repeat region (5-8 bp). The six solid-colour homopolymer loci highlighted were found to be statistically enriched hotspots for frameshift indels in tumours. Data from Gorelick et al., 2021. C—Space-filling model of respiratory complex I with main reactions/functions annotated. D—Space-filling model of respiratory complex I with internally localised, buried MT-ND5 and its interaction with NDUFB8 protein that is exposed to solvent on both the upper and lower surfaces of complex I highlighted.

FIG. 2 shows how recurrent mutations in tumour Mt-Nd5 were modelled. A—Schematic of mouse mitochondrial genome indicating sites to which DdCBEs are targeted. B—Schematic of TALE DNA binding domains for DdCBE pairs targeted to induce premature stop codons at m.11,944 and m.12,436. C—Schematic of TALE-DdCBE library screening method employed. Briefly, candidate pairs were cloned into vectors co-expressing fluorescent marker proteins, allowing sorting of transfected B78 murine melanoma cells by fluorescence-activated cell sorting (FACS). Cells are then assessed for mutagenic efficiency by sequencing.

FIG. 3 shows how recurrent mutations in tumour Mt-Nd5 were generated. A—Heteroplasmy of cells transfected with indicated constructs as determined by pyrosequencing. N transfections indicates cells transfected and recovered once, or four times sequentially. B—mtDNA copy number of cells in FIG. 4A as measured by droplet digital PCR (ddPCR). C—Western blot analysis of marker proteins for the respiratory chain complexes. Complex I (Ndufb8), complex II (Sdhb), complex III (Uqcrc2), complex IV (Mt-Col) and complex V (Atp5a). Ponceau is shown as a loading control.

FIG. 4 shows how recurrent mutations in tumour Mt-Nd5 were generated. A—Blue native (BN) PAGE and blotting for respiratory chain complexes using antibodies as in FIG. 4C. In gel activity of complex I and complex II following BN PAGE is also shown, along with Coomassie loading control. B—Basal oxygen consumption rate (OCR) of cells as assessed by Seahorse. C—Energy charge state analysis of cells using metabolite abundance data of AMP, ADP and ATP derived from mass spectrometric metabolomic measurements. D—NAD+:NADH ratio as calculated using metabolite abundance data derived from mass spectrometric metabolomic measurements.

FIG. 5 shows the impact of mt-Nd5 mutations on cellular energetics and metabolism. Metabolite abundances derived from mass spectrometric metabolomic measurements of high VAF mutant cells are plotted against each other, revealing consistent metabolic changes due to the two distinct truncating mutations in Mt-Nd5.

FIG. 6 shows that glutamine tracing reveals an increase in MDH1-derived malate abundance in the cytosol. A—heatmap indicating significantly elevated abundance of specific metabolites related to the tricarboxylic acid (TCA) cycle, urea cycle and fumarate adducts. B—schematic of labelling fate for 1-¹³C glutamine. C—abundance of malate m+1. D—abundance of argininosuccinate m+1. E—abundance of a-ketoglutarate (a-KG) m+1. F—abundance of aconitate m+1. G—abundance of aspartate m+1.

FIG. 7 shows that glucose tracing suggests an increase in malate abundance via reverse MDH2 flux. A—schematic of labelling fate for U-¹³C glucose. B—ratio of malate m+3:citrate m+3 derived from mass spectrometric analyses. C—ratio of citrate m+3:aconitate m+3 derived from mass spectrometric analyses. D—ratio of citrate m+3:pyruvate m+3 derived from mass spectrometric analyses.

FIG. 8 shows that MDH1 may mediate the increase in glycolytic intermediates in mutant cells. A—heatmap representation of glycolytic intermediate abundance as detected by mass spectrometry. B—heatmap representation of glycolytic intermediate abundance upon siRNA mediated depletion of MDHI1, as detected by mass spectrometry. C—western blot analysis demonstrating knockdown of MDH1 as compared with scrambled siRNA control.

FIG. 9 shows the abundance of specific metabolites in cells treated with siRNA. A —schematic of labelling fate for 4-²H₁ glucose. B—malate+1 abundance in cells treated with scrambled siRNA derived from mass spectrometric analyses. C—lactate+1 abundance in cells treated with scrambled siRNA derived from mass spectrometric analyses. D—NADH+1 abundance in cells treated with scrambled siRNA derived from mass spectrometric analyses. E—malate+1 abundance in cells treated with siRNA to MDHI1 derived from mass spectrometric analyses. F—lactate+1 abundance in cells treated with siRNA to MDH1 derived from mass spectrometric analyses.

FIG. 10 shows the impact on cancer metabolism in the context of the Krebs cycle (A and B).

FIG. 11 shows the experimental set up used to analyse the situation in vivo.

FIG. 12 shows the impacts in vivo. A—survival curve of mice engrafted with subcutaneous tumours. Humane endpoint is reached when tumours measure 15 mm in any dimension. n=12 mice per genotype. B—dissected tumour weight. n=10-12 tumours per genoptype. C—difference in mean heteroplasmy between injected cancer cells and resulting bulk tumour heteroplasmy measurement, as determined by pyrosequencing. n=11-12 per genotype. D —mtDNA copy number analysis of bulk tumour as determined by ddPCR. n=10-12 per genotype. E—metabolite abundances of tumours with indicated genotypes. n=7-10 per genotype.

FIG. 13 shows tumour transcriptional profiling. A—volcano plot presenting differential gene expression between bulk transcriptomics from mtDNA wild-type and 60% VAF m.11,944 G>A tumours. B—PCA plot of samples compared in A. Each point is a single tumour. C—volcano plot presenting differential gene expression between bulk transcriptomics from mtDNA wild-type and 40% m.11,944 G>A VAF tumours. D—PCA plot of samples compared in C. Each point is a single tumour. E—volcano plot presenting differential gene expression between bulk transcriptomics from 40% VAF m.11,944 G>A and 60% VAF m.11,944 G>A tumours. F—PCA plot of samples compared in E. Each point is a single tumour.

FIG. 14 shows differentially expressed genes; bulk tumour GSEA—wild-type vs VAF >50%. A—summary of differentially expressed genes between mtDNA wild-type and 60% VAF m.11,944 G>A tumours. B—provides the same information as FIG. 13A. C—Significant hits from gene set enrichment analysis (GSEA) of differentially expressed genes.

FIG. 15 shows differentially expressed genes; bulk tumour GSEA—VAF <50% vs VAF >50%. A—summary of differentially expressed genes between 40% VAF and 60% VAF m.11,944 G>A tumours. B—provides the same information as FIG. 13C. C—Significant hits from gene set enrichment analysis (GSEA) of differentially expressed genes.

FIG. 16 shows the reshaped immune microenvironment in mtDNA mutant tumours. A—Proportion of natural killer (NK) cells detected in tumours following dissociation and flow cytometry. n=4-8. B—Proportion of tumour-associated macrophages (TAMs) detected in tumours following dissociation and flow cytometry. n=9-14. C—Proportion of immature monocytes detected in tumours following dissociation and flow cytometry. n=10-14. D—Flow cytometry gating strategy for defining TAMs and monocyte populations. E—Flow cytometry gating strategy for defining natural killer cells.

FIG. 17 shows scRNAseq profiling of tumours defines altered immune populations. A—UMAP representation of Seurat clustered single cell RNA sequencing (scRNAseq) data of >100,000 cells harvested from whole, dissociated mtDNA wild-type and 60% VAF m.12,436G>A tumours. n=3 of each genotype. B—Cell type assignments of scRNAseq data based on CellRanger. C—relative proportions of intermediate monocytes in cluster 3. D —relative proportions of NK cells in cluster 11. E—relative proportions of macrophages in cluster 6. F—relative proportions of classical monocytes in cluster 13. G—relative proportions of interferon stimulatory gene expressing immune cells (ISG Expr. Imm. cells) in cluster 7. H—relative proportions of CD4+NK-like T cells in cluster 24.

FIG. 18 shows scRNAseq profiling of tumours described in FIG. 17. A—GSEA results across all defined clusters for hallmark geneset Interferon gamma response. B—GSEA results across all defined clusters for hallmark geneset Interferon alpha response.

FIG. 19 shows VAF >50% mtDNA mutant melanoma responds to PD1 immune checkpoint blockade. A—schematic of experimental timeline. Cells are engrafted at DO. Once the tumour is palpable, typically at day 7, mice are dosed with intraperitoneal (IP) anti-PD1 monoclonal antibody every 3 days until the experiment concludes at day 21, mice are sacrificed and tumours excised. B—weight of excised tumours from mice bearing tumours with indicated genotype treated with anti-PD1 antibodies as defined in A. n=4-5. C—representative images of excised tumours from B. D—weight of excised tumours from mice bearing tumours with indicates genotype treated with anti-CTLA4 antibodies as defined in A. n=4-7. E —representative images of excised tumours from D.

FIG. 20 shows that this leads to therapeutic sensitivity in humans. A—Stratification of a metastatic melanoma patient cohort by mtDNA status. B— Response rate of patients to nivolumab by tumour mtDNA mutation status. P-values were determined using one-way ANOVA test with Sidak multiple comparisons test (C,F), one-tailed student's t-test (I) or chi-squared test (H) were applied. Error bars indicate SD. Measure of centrality is mean.

FIG. 21 shows that mitochondrial base editing leads to isogenic cell lines bearing two independent truncating mutations in mt-Nd5. A Schematic of TALE-DdCBE design employed. TALEs were incorporated into a backbone containing a mitochondria-targeting cassette, split-half DdCBE and uracil glycosylase inhibitor (UGI). B Schematic of the murine mtDNA. Targeted sites within mt-Nd5 are indicated. C TALE-DdCBE pairs used to induce a G>A mutation at mt.12,436 and mt.11,944. D Workflow used to produce mt-Nd5 mutant isogenic cell lines. E Heteroplasmy measurements of cells generated in D (n=6 separate wells were sampled). F Immunoblot of indicative respiratory chain subunits. Representative result is shown. G Assembled complex I abundance and in-gel activity. Representative result is shown. H mtDNA copy number (n=9 separate wells were sampled). I Basal oxygen consumption rate (OCR) (n=9-12 measurements (12 wells per measurement) were made). J Energy (adenylate) charge state (n=17 separate wells were sampled). K Proliferation rate of cell lines in permissive growth media. (n=12 separate wells were measured in three batches) L NAD+:NADH ratio (n=11-12 separate wells were measured). All P-values were determined using a one-way ANOVA test with (E, H-I, K) Sidak multiple comparisons test or (J,L) Fisher's LSD Test. Error bars indicate SD. Measure of centrality is mean.

FIG. 22 shows that mutant cells undergo a metabolic shift towards glycolysis due to cellular redox imbalance. A Heatmap of unlabelled steady-state abundance of select mitochondrial metabolites, arginine, argininosuccinate (AS) and terminal fumarate adducts succinylcysteine (succ. Cys) and succinicGSH (succ.GSH). B Labelling fate of ¹³C derived from 1-¹³C-glutamine. C Malate m+1 abundance, derived from 1-¹³C-glutamine with indicated treatment (n=6-11 separate wells were sampled). D Heatmap of unlabelled steady-state metabolite abundances for select intracellular glycolytic intermediates and extracellular lactate (ex. lactate). E Labelling fate of U-¹³C-glucose. F Abundance of U-¹³C-glucose derived lactate m+3 with indicated treatment (n=6-9 separate wells were sampled). G Labelling fate of ²H derived from 4-²H₁-glucose; mitoLbNOX not shown for clarity. H Malate m+1 abundance, derived from 4-²H₁-glucose with indicated treatment (n=5-16 separate wells were sampled). I IC₅₀ curves for 2-DG (n=4 separate wells measured per drug concentration). This was repeated 3 times and a representative result is shown. P-values were determined using a one-way ANOVA test with (A, D) Sidak multiple comparisons test or Fisher's LSD Test (C, F, H). Error bars indicate SD. Measure of centrality is mean.

FIG. 23 shows that tumour mtDNA mutations reshape the immune microenvironment. A Survival of C57/BL6 mice subcutaneously injected with indicated cells (n=5-12 animals per condition). B Tumour weight at endpoint (n=5-12 tumours per genotype). C Geneset enrichment analysis (GSEA) of bulk tumour RNA sequencing (RNAseq) data (n=5-6 tumours per genotype). Only genesets with adj. P-value <0.1 are shown. D GSEA of RNAseq obtained from Hartwig Medical Foundation (HMF) metastatic melanoma patient cohort. Cancers are stratified by mtDNA status into wild-type and mtDNA mutant with >50% variant allele frequency (VAF). E UMAP of seurat clustered whole tumour scRNAseq from indicated samples. F UMAP indicating cell type IDs. DC, dendritic cells. pDC, plasmacytoid dendritic cell. G GSEA of malignant cells identified in scRNAseq analysis. UMAPs coloured by GSEA score for: H interferon alpha response; I interferon gamma response; J inflammatory response; K IL2-Stat5 signalling. L Proportion of tumour resident neutrophils relative to total malignant and non-malignant cells (n=17 tumours). M UMAP coloured by GSEA for OXPHOS geneset. One-way ANOVA test with Sidak multiple comparisons test (B), Wilcoxon signed rank test (G-K) and two-tailed student's t-test (L-O) were applied. Error bars indicate SD (B) or SEM (L-O). Measure of centrality is mean. Box plots indicate interquartile range (J-M). NES: normalised expression score. In FIG. 23C, in each pair the top bar is m.11,944 and bottom bar is m.12,436.

FIG. 24 shows that mtDNA mutation-associated microenvironment remodelling sensitises tumours to checkpoint blockade. A Schematic of the experimental plan and dosing regimen for B78-D14 tumours with anti-PD1 monoclonal antibody (mAb). B Representative images of harvested tumours at day 21. C Tumour weights at day 21 (n=10-19 tumours per genotype). D Schematic of experimental plan and dosing regimen for Hcmel12 tumours with anti-PD1 mAb. E Representative images of harvested tumours at day 13. F Tumour weights at day 13 (n=7 tumours per genotype). G Stratification of a metastatic melanoma patient cohort by mtDNA status. H Response rate of patients to nivolumab by tumour mtDNA mutation status. One-way ANOVA test with Sidak multiple comparisons test (C), student's one-tailed t-test (F) or chi-squared test (H) were applied. Error bars indicate SD. Measure of centrality is mean.

FIG. 25 shows results of Mitochondrial base editors for two independent targets in mt-Nd5. A Immunoblot of DdCBE pair expression post-sort. αHA and αFLAG show expression of left (TALE-L) and right TALEs (TALE-R) respectively. Representative result is shown. B Off-target C>T activity of DdCBEs on mtDNA. Figure shows mutations detected at heteroplasmies >2% and is a measure of mutations detected relative to wild-type. These mutations likely do not impact our key observations as both models behave similarly across experiments.

FIG. 26 shows results of proteomic analysis of isogenic mt-Nd5 mutant cell lines reveals significant changes primarily in complex I genes. Volcano plot showing detected differences in protein abundance of A mt.12436^{60%} cells and B mt.11944^{60%} cells versus wild-type. Differences of p<0.05 and log₂ fold change >0.5 shown in red (n=3 separately collected cell pellets were measured per cell line). Heatmaps of protein abundances for C complex 1, D complex II, E complex III, F complex IV and G complex V nuclear and mitochondrial subunits. Wilcoxon signed rank test (A, B) and a one-way ANOVA test with Sidak multiple comparisons test (C-G) were applied.

FIG. 27 shows that mt.-Nd5 truncations alter the intracellular redox state with no significant impact on mitochondrial mRNA expression or membrane potential. A Expression of mitochondrial genes (n=12 separate cell pellets were sampled per genotype). B Measurements of the electrical component of the proton motive force, Δ^{ψ}, the chemical component of the proton motive force ΔpH and total protonmotive force, ΔP (n=4 separate wells were sampled per genotype). C GSH:GSSG ratio (n=6-12 separate wells were sampled per cell type). A high GSH:GSSG ratio represents a more reductive intracellular environment. D Mitochondrial NADH oxidation state (n=4 separate wells for sampled per genotype). All P-values were determined using a one-way ANOVA test with Sidak multiple comparisons test. Error bars indicate SD. Measure of centrality is mean. In FIG. 27A top bar in each group of three is m.11,944, middle bar is m.12,436, and bottom bar is wild-type.

FIG. 28 results of U-¹³C-glutamine labelling which show that a proportion of the increased malate abundance is derived from cytosolic reductive carboxylation of glutamine. A Labeling fate of ¹³C derived from U-¹³C-glutamine via oxidative decarboxylation versus reductive carboxylation of glutamine. B Malate m+3 abundance, derived from U-¹³C-glutamine (n=9 separate wells were sampled per genotype). C malate m+3:malate m+2 ratio, derived from U-¹³C-glutamine (n=9 separate wells were sampled per genotype). D AS m+3:AS m+2 ratio, derived from U-¹³C-glutamine (n=9 separate wells were sampled per genotype). All P-values were determined using a one-way ANOVA test with Sidak multiple comparisons test. Error bars indicate SD. Measure of centrality is mean.

FIG. 29 shows that increased malate abundance in the cytosol occurs at the level of MDH1 but is not directly due to global redox changes. A Labeling fate of ¹³C derived from 1-¹³C-glutamine which exclusively labels metabolites derived from the reductive carboxylation of glutamine. B Aconitate m+1 abundance, derived from 1-¹³C-glutamine (n=9 separate wells were sampled per genotype). C Aspartate m+1 abundance, derived from 1-¹³C-glutamine (n=9 separate wells were sampled per genotype). D AS m+1 abundance, derived from 1-¹³C-glutamine (n=9 separate wells were sampled per genotype). E Immunoblot of siRNA mediated depletion of Mdhl. Representative image shown. F Immunoblot of cytoLbNOX expression 36 hrs post-sort, detected using αFLAG. Representative image shown. G AS m+1 abundance, derived from 1-¹³C-glutamine with indicated treatment (n=6-12 separate wells were sampled per genotype per condition). All P-values were determined using a one-way ANOVA test with Sidak multiple comparisons test. Error bars indicate SD. Measure of centrality is mean.

FIG. 30 shows increased malate abundance in mutant cells is partially due to MDH2 reversal. A Labeling fate of ¹³C derived from U-¹³C-glucose. B Pyruvate m+3 abundance, derived from U-¹³C-glucose (n=7-8 separate wells were sampled per genotype). C Citrate m+2:pyruvate m+3 ratio, derived from U-¹³C-glucose (n=6-7 separate wells were sampled per genotype). D Malate m+3:citrate m+3 ratio, derived from U-¹³C-glucose (n=7-8 separate wells were sampled per genotype). E Immunoblot of mitoLbNOX expression 36 hrs post-transfection, detected using αFLAG. Representative image shown. All P-values were determined using a one-way ANOVA test with Sidak multiple comparisons test. Error bars indicate SD. Measure of centrality is mean.

FIG. 31 shows results of 4-²H₁-glucose tracing which demonstrates that shuttling of electrons between MDH1 and GAPDH drives aerobic glycolysis. A Lactate m+1 abundance, derived from 4-²H₁-glucose with indicated treatment (n=7-9 separate wells were sampled per genotype per condition). B NADH m+1 abundance, derived from 4-²H₁-glucose with indicated treatment (n=6-8 separate wells were sampled per genotype per condition). All P-values were determined using a one-way ANOVA test with Sidak multiple comparisons test. Error bars indicate SD. Measure of centrality is mean.

FIG. 32 shows that mutant cells demonstrate a heteroplasmy dose-dependent sensitivity to respiratory chain inhibitors. A IC₅₀ curve for metformin. IC₅₀ for wild-type=26.31±1.49 mM, for mt.12436^{60%}=16.60±2.43 mM, for mt.12436^{80%}=5.89±0.71 mM and for mt.11944^{80%}=22.93±0.70 mM B IC₅₀ curve for rotenone. IC₅₀ for wild-type=0.236±0.026 μM, for mt.12436^{60%}=0.235±0.035 μM, for mt.12436^{80%}=0.493±0.108 μM and for mt.11944^{60%}=0.205±0.033 μM and C IC₅₀ curve for oligomycin. IC₅₀ for wild-type=13.81±3.80 μM, for mt.12436^{60%}=13.52±3.32 μM, for mt.12436^{80%}=7.75±0.56 μM and for mt.11944^{80%}=13.54±3.32 μM (n=4 separate wells per drug concentration per genotype). This was repeated 3 times and a representative result is shown.

FIG. 33 shows that Allografted B78-D14 lineage tumours have no major macroscopic differences. Representative H&E sub-section of A wild-type, B m.12,436^{40%} and C m.12,436^{60%} tumours. D Change in detected heteroplasmy in bulk tumour samples (n=5-12 tumours per genotype). E Bulk tumour mtDNA copy number (n=4-13 tumours per genotype). F Heatmap of steady-state abundance of metabolically terminal fumarate adducts, succinylcysteine and succinicGSH, demonstrating that metabolic changes observed in vitro are preserved in vivo (n=12 tumours per genotype). All P-values were determined using a one-way ANOVA test with Sidak multiple comparisons test. Error bars indicate SD. Measure of centrality is mean.

FIG. 34 Bulk tumour transcriptional signatures show dose-dependent, heteroplasmy changes in immune-relevant transcriptional phenotypes. GSEA of bulk tumour RNAseq data (n=5-6 tumours per genotype) showing A mutant^{40%} versus wild-type and B mutant^{60%} versus mutant^{40%}. Only genesets with adj. p-value <0.1 are shown unless otherwise stated. Wilcoxon signed rank test applied. In each pair the top bar is m.11,944 and bottom bar is m.12,436.

FIG. 35 shows that Malignant cells were identified for scRNAseq analysis as aneuploid cells with low or nil Ptprc (CD45) expression and high epithelial score. UMAP indicating A Ptprc expression, B epithelial score and C aneuploidy as determined by copykat prediction. These criteria were employed as the B78 cells lack distinct transcriptional signatures. In each pair the top bar is m.11,944 and bottom bar is m.12,436.

FIG. 36 shows that mutant cells did not have significant changes in transcriptional signatures in vitro. A Significantly co-regulated transcripts from combined 60% mutant cells versus wild-type (n=12 cell pellets were sampled per genotype). Volcano plot showing differences in gene expression of A mt.12436^{60%} cells and B mt.11944^{60%} cells versus wild-type. Differences of p <0.05 and log 2 fold change >1 shown in red (n=12 separate wells were sampled). Wilcoxon signed rank test applied.

FIG. 37 shows results of scRNAseq analyses which reveal distinct alterations in the tumour immune microenvironment of mtDNA mutant tumours. Proportion of tumour resident: A immature monocytes; and B CD4+ T-cells relative to the total malignant and non-malignant cells (n=3-7 tumours per genotype). C UMAP coloured by GSEA NES score for allograft rejection geneset. Proportion of tumour resident: D CD4+ T cells; and E natural killer (NK) cells relative to the total malignant and non-malignant cells (n=3-7 tumours per genotype). F Relative PD-L1 expression within each cell (n=3-7 tumours per genotype). One-way ANOVA test with Wilcoxon signed rank test (A) and two-tailed student's t-test (A-B, D-E) were applied. Error bars indicate SEM. Measure of centrality is mean. Box plots indicate interquartile range (A-B, D-E). NES: normalised expression score. DC, dendritic cell.

FIG. 38 shows that remodelling of the tumour microenvironment in mutant cells sensitizes tumours to checkpoint blockade. Harvested tumour weight at day 21 (n=5-15 tumours per genotype). One-way ANOVA test with Sidak multiple comparisons test was applied. Error bars indicate SD. Measure of centrality is mean. In figure a, b, d and e, the order of bars in the graphs, left to right is control (ctrl), ND5^{60%}, and ND5^{80%}.

FIG. 39 shows that HcMel12 mutant cells recapitulate the cellular and metabolic phenotypes observed in B78-D14 cells. A Heteroplasmy changes upon subsequent transfection of melanoma cell lines (n=3 separate cell pellets per genotype). B Immunoblot of indicative respiratory chain subunits. Representative result is shown. C mtDNA copy number (n=12 separate wells per genotype). D Basal oxygen consumption rate (OCR) (n=6 measurements (12 wells per measurement) per genotype). E Proliferation rate of cell lines in permissive growth media (n=3 separate wells per genotype) F Energy (adenylate) charge state (n=9 separate wells per genotype). G NAD+:NADH ratio (n=9 separate wells per genotype). H GSH:GSSG ratio (n=8-9 separate wells per genotype). I Heatmap of unlabelled steady-state abundance of select mitochondrial metabolites, arginine, argininosuccinate (AS) and terminal fumarate adducts succinylcysteine (succ. Cys) and succinicGSH (succ.GSH) (n=9 separate wells per genotype). J Heatmap of unlabelled steady-state metabolite abundances for select intracellular glycolytic intermediates and extracellular lactate (ex. lactate) (n=9 separate wells per genotype). P-values were determined using a one-way ANOVA test with (C-D) Sidak multiple comparisons test, Fisher's LSD Test (E) or (F-J) a one-tailed student's t-test. Error bars indicate SD. Measure of centrality is mean.

FIG. 40 shows that untreated Hcmel12 lineage tumours recapitulate B78-D14 lineage. A Survival of C57/BL6 mice subcutaneously injected with indicated cells (n=9-10 animals per genotype). B Tumour weight at endpoint (n=9-10 tumours per genotype). C Change in detected heteroplasmy in bulk tumour samples (n=9 tumours per genotype). D Bulk tumour mtDNA copy number (n=9 tumours per genotype). E Heatmap of steady-state abundance of metabolic terminal fumarate adducts, succinylcysteine and succinicGSH, demonstrating that metabolic changes observed in B78 mutant tumours are preserved in vivo (n=9 tumours per genotype). P-values were determined using a one-way ANOVA test with (B,D) Sidak multiple comparisons test or student's one-tailed t-test (E). Error bars indicate SD. Measure of centrality is mean.

FIG. 41 shows that constitutive expression of cytoLbNOX phenocopies metabolic changes observed in mt-Nd5 mutant cells. A. Immunoblot of cytoLbNOX expression in clonal population, detected using αFLAG. Representative image shown. B. Immunoblot of indicative respiratory chain subunits. Representative result is shown. C. mtDNA copy number (n=9 separate wells per genotype). D Basal oxygen consumption rate (OCR) (n=9-15 measurements (6 wells per measurement) per genotype) A significant decrease is observed in HcMel12 cytoLbNOX, akin to the decrease in basal OCR measured in m.12,436^{80%} cells. E. NAD+:NADH ratio (n=11-12 separate wells per genotype). F. Heatmap of metabolite abundance of glucose m+3, lactate m+3, pyruvate m+3, and terminal fumarate adducts succinylcysteine (succ. Cys) and succinicGSH (succ.GSH) in U-¹³C-glucose labelling of B78 cells. B78 wild-type cells were transiently transfected with cytoLbNOX and metabolites were extracted 3 days post-sort. A significant increase in lactate abundance was observed in cytoLbNOX-expressing cells mimicking that observed in m.12,436^{80%} cells. (n=9-13 separate wells per genotype). All P-values were determined using a one-paired student's t-test. Error bars indicate SD. Measure of centrality is mean.

FIG. 42 Hcmel12 mutant and cytoLbNOX tumours showed decreased neutrophil and increased CD4+ T-cell infiltration relative to wild-type tumours. A Gating strategy for Zombie+ live cells. B Gating strategy for neutrophils in tumours, lymph nodes and spleens. C Gating strategy for CD4+ T-cells, CD8+ T-cells, NK T-cells and macrophages in tumours, lymph nodes and spleens. Abundance of specific immune cells in D tumour, E tumour draining lymph node and F spleen in untreated mice (n=4-8 samples per genotype). Tissue was harvested on day 13. Natural Killer T-cells: CD4−CD8−NK1.1+. Macrophages: Cd11b+Ly6C−F4/80+. Neutrophils: CD11b+Ly6C+Ly6G+. All P-values were determined using a one-way ANOVA test with Fisher's LSD Test. Measure of centrality is mean.

FIG. 43 Anti-PD1 response is dependent on the abundance of tumour-resident neutrophils in syngeneic models of Hcmel12 melanoma. A Schematic of the experimental plan and dosing regimen for Hcmel12 tumours with anti-PD1 monoclonal antibody (mAb) and either G-CSF or anti-Ly6G. B Tumour weight of untreated mice compared to mice treated with G-CSF or anti-Ly6G (n=7-8 tumours per genotype). Log 2 fold change of tumour neutrophils in untreated and treated mice relative to untreated control for C G-CSF and D anti-Ly6G (n=4-8 samples per genotype). Tumour weight of mice treated with anti-PD1 or antiPD1 and E G-CSF or F anti-Ly6G (n=7-8 tumours per genotype). Neutrophils: CD11b+Ly6C+Ly6G+. All P-values were determined using a one-way ANOVA test with Fisher's LSD Test. Error bars indicate SD. Measure of centrality is mean.

FIG. 44 Hcmel12 mutant and cytoLbNOX tumours show differential sensitivity to immune checkpoint inhibitors (also referred to herein as immune checkpoint blockage, or ICB). A Schematic of experimental plan and dosing regimen for Hcmel12 tumours with anti-PD1, anti-PDL1 or anti-CTLA4 mAbs. B Representative images of harvested tumours at day 13 for each drug regimen. C Tumour weights at day 13 (n=10-12 tumours per genotype) for each drug regimen. Survival of C57BL/6 mice subcutaneously injected with indicated cells (n=10-15 animals per genotype) on sustained anti-PD1 therapy. Only tumours that hit endpoint of 15 mm shown for cytoLbNOX. E Tumour weight at endpoint for mice on sustained anti-PD1 therapy (n=3-15 tumour per genotype). Tumour volume changes recorded from injection date for F wild-type and m.12436^{80%} (n=15 tumours per genotype) and G cytoLbNOX tumours (n=10 tumours per genotype) on sustained anti-PD1. One-way ANOVA with Sidak multiple comparisons test (C,E) or Log-rank (Mantel-Cox) test (D) were applied. Tumour volume calculated as 0.5*L*W² based on calliper measurements. Error bars indicate SD. Measure of centrality is mean.

FIG. 45 Immunogenic 4434 tumours maintain differential anti-PD1 sensitivity. A Schematic of the experimental plan and dosing regimen for 4434 tumours with anti-PD1 mAb. B Representative images of treated tumours at day 20 and untreated tumours at endpoint. C Tumour weights at day 21 (n=13 tumours per genotype). All P-values were determined using a one-tailed student's t-test. Error bars indicate SD. Measure of centrality is mean.

FIG. 46 Wild-tumours implanted on the opposite flank to mitochondrial mutant or cytoLbNOX tumours are sensitised to anti-PD1. A Schematic of tumour injection sites and dosing regimen for Hcmel12 tumours with anti-PD1 mAb. Representative images of B culled mice and C harvested tumours at day 13 for each condition. D Tumourweights at day 13 (n=6-10 tumours per genotype). E Wild-type tumour weights at day 13 with respective flanking tumour genotype (n=8-11 wild-type tumours). F Heatmap of circulating immune populations in blood sampled on day 11. NK cells: CD4−CD8−NK1.1+. Neutrophils: CD11b+Ly6C+Ly6G+. Monocytes: CD11b+Ly6C+F4/80−. Conventional dendritic cells (cDCs): CD11c+MHCII+. All P-values were determined using a one-way ANOVA test with Fisher's LSD Test. Error bars indicate SD. Measure of centrality is mean.

FIG. 47 Flow cytometry of treated contralateral tumours reveal increases in CD4+ T-cells. Proportion of A CD4+ T-cells, B NK T-cells, C CD8+ T-cells, D tumour-associated macrophages (TAMs), E neutrophils and F monocytes (n=6-12 tumours per condition). All P-values were determined using a one-way ANOVA test with Fisher's LSD Test. Error bars indicate SD. Measure of centrality is mean.

FIG. 48 Tumour weight and growth rate of wild type tumours and complex IV mutated tumours.

FIG. 49 Effects of anti-PD1 treatment on wild type tumours and complex IV mutated tumours. The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

FIG. 50 A) Endpoint tumour weight of tumours from C57/BL6 mice subcutaneously injected with indicated tumour cell genotype (n=9-18 animals per genotype). Measure of centrality is the mean. Error bars indicate SD. B) Survival of C57/BL6 mice subcutaneously injected with indicated cells (n=9-18 animals per genotype). Log rank (Mantel-Cox) test applied.***P=<0.001. C) Heatmap of unlabelled steady-state abundance of select metabolites from endpoint tumours of indicated genotype grown subcutaneously in C57/BL6 animals. Succ. cys, succinylcysteine. n=5-42 tumours per genotype. All P-values were determined using a one-way ANOVA with Fisher's LSD test.*P=<0.05. D) Immunoblot analysis of endpoint whole tumour protein extracts from indicated genotype grown subcutaneously in C57/BL6 animals. E) Quantification of pSTAT1 levels across indicated tumour genotypes. n=3 tumours per genotype. All P-values were determined using a one-way ANOVA with Fisher's LSD test. Measure of centrality is the mean. Error bars indicate SD.*P=<0.05, **P=<0.01, ***P=<0.001.

FIG. 51. There are no common bulk tumour metabolite changes across conditions. Heatmap of metabolite abundance changes relative to wild-type tumours for respective tumour lineages (n=6-38 tumours per genotype). One-tailed student's t-test (B78 and 4434) or one-way ANOVA with Sidak multiple comparisons test (Hcmel12) were applied. Error bars indicate SD. Measure of centrality is mean.

Various aspects of the invention are described in further detail below.

### DETAILED DESCRIPTION

The present disclosure is based on the inventors' identification of a subpopulation of cancer or pre-cancer patients that respond more favourably to treatment with an immune checkpoint inhibitor (such as a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and/or KIR inhibitor). Based on the data provided in the Examples below, the inventors conclude that these patients have an altered cancer or pre-cancer lactate to glucose ratio, and therefore an altered cancer or pre-cancer redox status (indicative of Warburg-like metabolic shift). This altered redox status results in a change to the overall tumour microenvironment such that different ratios of immune cells are present within the cancer or pre-cancer. Specifically, the inventors observed that cancers or pre-cancers with an altered redox status, for example altered lactate to glucose ratio have increased numbers of Natural Killer (NK) cells, monocytes, CD4+NK-like T cells, and interferon-stimulated gene (ISG) expressing immune cells, and decreased numbers of macrophages.

Accordingly, in one aspect, the present invention provides an agent that alters the redox status (for example alters the lactate to glucose ratio) lactate to glucose ratio of a cancer or a pre-cancer for use in sensitising a subject having cancer or pre-cancer to an immune checkpoint inhibitor. In one example, the agent alters the redox status (for example alters the lactate to glucose ratio) in the interstitial fluid of the cancer or a pre-cancer.

In a related aspect, the present invention provides a method of sensitising a subject having a cancer or a pre-cancer to an immune checkpoint inhibitor, comprising exposing the subject to an agent that alters the redox status (for example alters the lactate to glucose ratio) in the cancer or pre-cancer. In one example, the agent alters the redox status (for example alters the lactate to glucose ratio) in the interstitial fluid of the cancer or a pre-cancer.

In a further aspect, the invention provides an immune checkpoint for use in treating a subject having a cancer or a pre-cancer, wherein the subject has been exposed to an agent that alters the redox status (for example alters the lactate to glucose ratio) in the cancer or pre-cancer. In one example, the agent alters the redox status (for example alters the lactate to glucose ratio) in the interstitial fluid of the cancer or a pre-cancer.

The invention further provides a method of treating a cancer or a pre-cancer in a subject, comprising administering an immune checkpoint inhibitor to the subject, wherein the subject has been exposed to an agent that alters the redox status (for example alters the lactate to glucose ratio) in the cancer or pre-cancer. In one example, the agent alters the redox status (for example alters the lactate to glucose ratio) in the interstitial fluid of the cancer or a pre-cancer.

In a further aspect, the invention provides a method of treating a cancer or a pre-cancer in a subject, comprising:
(i) exposing the subject to an agent that alters the redox status (for example alters the lactate to glucose ratio) in the cancer or pre-cancer; and
(ii) administering an immune checkpoint inhibitor to the subject. In one example, the agent alters the redox status (for example alters the lactate to glucose ratio) in the interstitial fluid of the cancer or a pre-cancer.

As used herein, the term “sensitising”, in the context of a treatment with an immune checkpoint inhibitor, refers to increasing the sensitivity or reducing the resistance of a subject's cancer or pre-cancer to an immune checkpoint inhibitor treatment. Sensitisation may be of a cancer or pre-cancer that was not sensitive to an immune checkpoint inhibitor treatment prior to the subject being exposed to the agent, or increasing the sensitivity of a cancer or pre-cancer that was sensitive (at least partially) to an immune checkpoint inhibitor treatment prior to the subject being exposed to the agent. A subject (or a subject's cancer or pre-cancer) that has been sensitised is more likely to respond favourably to, or benefit from, such a treatment. In other words the immune checkpoint inhibitor treatment is likely or expected to have a therapeutic effect on the subject's cancer or pre-cancer, and/or to improve the therapeutic effect on the subject's cancer or pre-cancer. Such a therapeutic effect may include a clinical improvement of the cancer or pre-cancer in a subject with this disease or condition. A clinical improvement may be demonstrated by an improvement of the pathology and/or symptoms associated with the cancer or pre-cancer. Suitably, therapeutic effect may be demonstrated by preventing the development of the cancer or pre-cancer in a subject, slowing or halting the progression of the cancer or pre-cancer in the subject, or reversing the cancer or pre-cancer. Suitably, the cancer or pre-cancer may be reversed partially, or completely. Clinical improvement of the pathology may be demonstrated by one or more of the following: reduced cancer or pre-cancer biomarker levels in the subject, reduced cancer or pre-cancer cell number in the subject, increased time to regrowth of cancer upon stopping of treatment, prevention or delay of pre-cancer development into cancer, prevention of regrowth of cancer upon stopping treatment, decreased tumour invasiveness, reduction or complete elimination of metastasis, increased cancer cell differentiation, or increased survival rate. Other suitable indications of clinical improvement in the pathology will be known to the skilled person. It will be appreciated that indications of clinical improvement of the pathology will vary depending on the type of cancer. Clinical improvement of symptoms associated with cancer may be, but are not limited to, partial or complete alleviation of pain and/or swelling, increased appetite, reduced weight loss, and/or reduced fatigue.

Suitably, sensitised subjects may have about a 1.25-fold, 1.50-fold, 1.75-fold, 2-fold, 2.25-fold, 2.5-fold, 2.75-fold, 3-fold or more, increased likelihood of a PD-1 inhibitor and/or a PD-L1 inhibitor treatment having a therapeutic effect as compared to subjects that have not been sensitised.

As used herein, the term “cancer” refers to a large family of diseases which involve abnormal cell growth with the potential to invade or spread to other parts of the body due to the presence of “cancerous cells”. The cancerous cells may form a subset of neoplasms or tumours. A neoplasm or tumour is a group of cells that have undergone unregulated growth, and will often form a mass or lump, but may be distributed diffusely. The tumour or neoplasm may comprise a mixture of cancerous cells (and/or pre-cancerous cells) and healthy (i.e. non-cancerous) cells. The term “tumour” as used herein, encompasses the cancerous and/or pre-cancerous cells, healthy cells (for example stromal cells), as well as the tumour microenvironment which comprises immune cells and the interstitial fluid. The immune cells in the tumour microenvironment may be refers to as the “immune microenvironment” of the tumour.

The term “interstitial fluid” refers to the fluid that occupies the space between the cells (healthy, cancerous, and/or pre-cancers) of the tumour. The interstitial fluid may comprise, metabolites, ions, signalling molecules, proteins, extracellular vesicles, and/or other components secreted by the cells of the tumour and immune cells present therein. As it will be appreciated by the person skilled in the art, a change in the cells of the tumour may lead to change in the interstitial fluid. Merely by way of example, a change in the metabolic status of the cells of the tumour may result in an alteration of the metabolites in the interstitial fluid. As shown by the present inventors, such a change in the metabolic status of the cells of the tumour may alter the tumour microenvironment, for example by altering the immune cell populations within the tumour.

“Cancer cells” may be defined by one or more of the following characteristics: reduced differentiation, self-sufficiency in growth signalling, insensitivity to anti-growth signals, evasion of apoptosis, enabling of a limitless replicative potential, induction and sustainment of angiogenesis, and/or activation of metastasis and invasion of tissue.

A cancer may be a solid cancer or a liquid cancer. Suitably, a cancer may be selected from the group consisting of: a childhood cancer, haematological cancer, and a myeloid cancer. Suitably, a childhood cancer may be selected from the group consisting of: leukaemia, brain cancer, spinal cord cancer, neuroblastoma, Wilms tumour, lymphoma (such as Hodgkin and non-Hodgkin), rhabdomyosarcoma, retinoblastoma, and bone cancer (such as osteosarcoma and Ewing sarcoma). Suitably, the cancer may be a skin cancer. Suitably, the skin cancer may be selected from the group consisting of melanoma, basal cell carcinoma, squamous cell carcinoma, Kaposi's sarcoma, and keratoacanthoma. More suitably, the skin cancer may be melanoma. The present application provides examples relating to melanoma. However, the skilled person would appreciate that the aspects of the present invention may apply to other cancers. Nevertheless, the aspects of the present invention may work particularly well in the context of melanoma.

As used herein, “pre-cancer” or a “pre-cancerous condition” is an abnormality that has the potential to become cancer (such a cancer mentioned hereinabove), wherein the potential to become cancer is greater than the potential if the abnormality was not present, i.e., was normal. Examples of pre-cancer include but are not limited to adenomas, hyperplasias, metaplasias, dysplasias, benign neoplasias (benign tumours), premalignant carcinoma in situ, and polyps. In one example, the pre-cancer is a pre-cancer tumour. Such a tumour may comprise pre-cancerous and healthy cells.

As will be clear to a person skilled in the art, the “cancer” and/or “pre-cancer” may be referred to as “a tumour”.

Suitably, in the context of the present disclosure, the cancer or pre-cancer may have a deleterious mitochondrial DNA (mtDNA) mutation load. Typically, in the context of the disclosure, a subject that is more likely to benefit from sensitisation as described herein will have a cancer or pre-cancer with a low deleterious mitochondrial DNA (mtDNA) mutation load. In this context, sensitisation may mimic the metabolic changes seen in subjects with a high deleterious mitochondrial DNA (mtDNA) mutation load (see Examples below). Notwithstanding this, sensitisation as described herein may also be beneficial to subjects with a cancer or pre-cancer having a high deleterious mitochondrial DNA (mtDNA) mutation load (for example to further increase the therapeutic effect of a PD-1 inhibitor and/or PD-L1 inhibitor treatment).

Suitably, the cancer or pre-cancer may have a low deleterious mitochondrial DNA (mtDNA) mutation load. In the context of the present disclosure, a low deleterious mitochondrial DNA (mtDNA) mutation load may be a mutation load of less than 50% when determined solely or substantially only on cancer or pre-cancer cells. For example, a low deleterious mitochondrial DNA (mtDNA) mutation load may be a mutation load of less than 40%, or less than 30%, when determined solely or substantially only on cancer or pre-cancer cells. More suitably, a low deleterious mitochondrial DNA (mtDNA) mutation load may be a mutation load of less than 20% when determined solely or substantially only on cancer or pre-cancer cells. Suitably, in the context of the present disclosure, a low deleterious mitochondrial DNA (mtDNA) mutation load may be a mutation load of less than 30%, less than 20%, less than 10%, when determined on a sample from the subject. It will be appreciated by a person skilled in the art that a sample will typically comprise a mixture of cancerous cells (and/or pre-cancerous cells) and healthy cells, found within the tumour.

Suitably, the cancer or pre-cancer may have a high deleterious mitochondrial DNA (mtDNA) mutation load. In the context of the present disclosure, a high deleterious mitochondrial DNA (mtDNA) mutation load may be a mutation load of at least 50% or at least 60%, or more, when determined solely or substantially only on cancer or pre-cancer cells. For example, a high deleterious mitochondrial DNA (mtDNA) mutation load may be a mutation load of at least 70%, at least 80% or more, when determined solely or substantially only on cancer or pre-cancer cells. More suitably, a high deleterious mitochondrial DNA (mtDNA) mutation load may be a mutation load of at least at least 60% when determined solely or substantially only on cancer or pre-cancer cells. Suitably, in the context of the present disclosure, a high deleterious mitochondrial DNA (mtDNA) mutation load may be a mutation load of at least 30%, at least 40%, at least 50% or more, when determined on a sample from the subject. It will be appreciated by a person skilled in the art that a sample will typically comprise a mixture of cancerous cells (and/or pre-cancerous cells) and healthy cells, found within the tumour.

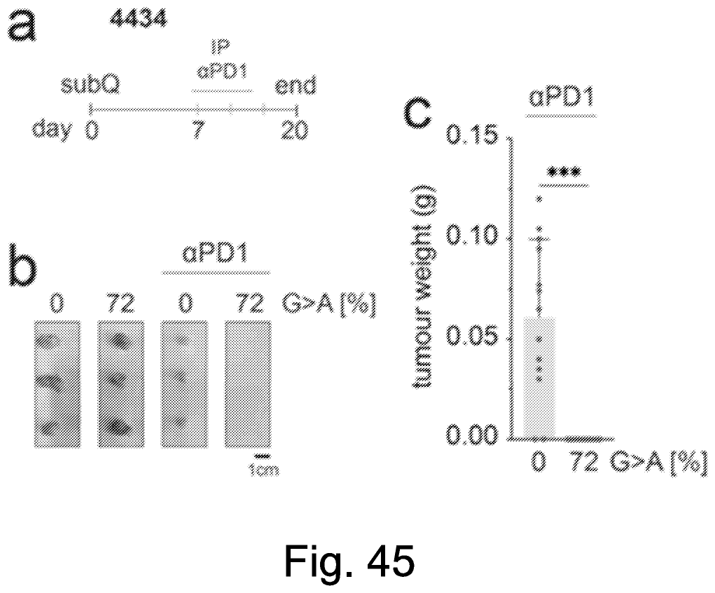

Suitably, the cancer or pre-cancer may have a high nuclear mutation burden. Such a cancer may be referred to as TMB-H (tumour mutation burden-high) cancer. Suitably, the TBM-H cancer may be a solid cancer. Suitably, the solid cancer may be selected from the group consisting of skin cancer (such as melanoma), lung cancer, liver cancer, kidney cancer, and head and neck cancer. Such cancers are generally found to have better sensitivity to immune checkpoint inhibitors, and the present inventors believe that by treating these cancers with agents that alter the redox status (for example alter the lactate to glucose ratio), the sensitivity to checkpoint inhibitors may be further increased. In fact, as shown in FIG. 45, cancers with an altered redox status (for example altered lactate to glucose ratio) due to mtDNA mutations were found to completely regress upon treatment with a checkpoint inhibitor (such as anti-PD1 antibody).

Suitably, the cancer or pre-cancer may have a high nuclear mutation burden and a hight mtDNA mutation load.

In the context of the present disclosure, the term “subject” includes humans and mammals (e.g., mice, rats, pigs, cats, dogs, and horses). In suitable embodiments, subjects are mammals, particularly primates, especially humans. In suitable embodiments, subjects are livestock such as cattle, sheep, goats, cows, swine, and the like; poultry such as chickens, ducks, geese, turkeys, and the like; and domesticated animals particularly pets such as dogs and cats. In certain embodiments (e.g., particularly in research contexts) subject mammals will be, for example, rodents (e.g., mice, rats, hamsters), rabbits, primates, or swine such as inbred pigs and the like. Herein, the terms, “patients” and “subjects” may be used interchangeably.

Immune checkpoint inhibitors are agents that inhibit proteins or peptides (e.g. immune checkpoint proteins) which are blocking the immune system, e.g., from attacking cancer cells. In some examples, the immune checkpoint protein blocking the immune system prevents the production and/or activation of T cells. An immune checkpoint inhibitor can be an antibody or antigen-binding fragment thereof, a protein, a peptide, a small molecule, or combination thereof. Typically, the inhibitor interacts directly to a target immune checkpoint protein (or its ligand, where appropriate) and thereby disrupts its function/biological activity. For example, it may bind directly to a target immune checkpoint protein (or its ligand, where appropriate). In one example, direct binding to a target immune checkpoint protein (or its ligand, where appropriate) inhibits, prevents or reduces the formation of protein complexes which are needed for immune checkpoint protein function/biological activity.

PD-1 inhibitors, PD-L1, and PD-L2 inhibitors are a group of checkpoint inhibitors that block or reduce the activity of PD-I, PD-L1 and PD-L2 immune checkpoint proteins. A review describing immune checkpoint pathways and the blockade of such pathways with immune checkpoint inhibitor compounds is provided by Pardoll in Nature Reviews Cancer (April, 2012). Immune check point inhibitor compounds display anti-tumour activity by blocking one or more of the endogenous immune checkpoint pathways that downregulate an anti-tumour immune response. The inhibition or blockade of an immune checkpoint pathway typically involves inhibiting a checkpoint receptor and ligand interaction with an immune checkpoint inhibitor compound to reduce or eliminate the signal and resulting diminishment of the anti-tumour response.

The immune checkpoint inhibitor compound may inhibit the signaling interaction between an immune checkpoint receptor and the corresponding ligand of the immune checkpoint receptor. The immune checkpoint inhibitor compound can act by blocking activation of the immune checkpoint pathway by inhibition (antagonism) of an immune checkpoint receptor (some examples of receptors include CTLA-4, PD-1, and NKG2A) or by inhibition of a ligand of an immune checkpoint receptor (some examples of ligands include PD-L1 and PD-L2). In such examples, the effect of the immune checkpoint inhibitor compound is to reduce or eliminate down regulation of certain aspects of the immune system anti-tumour response in the tumour microenvironment.

The immune checkpoint receptor programmed death 1 (PD-1) is expressed by activated T-cells upon extended exposure to antigen. Engagement of PD-1 with its known binding ligands, PD-L1 and PD-L2, occurs primarily within the tumour microenvironment and results in downregulation of anti-tumour specific T-cell responses. Both PD-L1 and PD-L2 are known to be expressed on tumour cells. The expression of PD-L1 and PD-L2 on tumours has been correlated with decreased survival outcomes.

Many PD-1 inhibitors and/or PD-L1 inhibitors are known in the art. In some examples, the PD-1 inhibitor and/or PD-L1 inhibitor is a small organic molecule (molecular weight less than 1000 daltons), a peptide, a polypeptide, a protein, an antibody, an antibody fragment, or an antibody derivative. In some embodiments, the inhibitor compound is an antibody. In some embodiments, the antibody is a monoclonal antibody, specifically a human or a humanized monoclonal antibody.

In some examples, the PD-1 inhibitor is an anti-PD-1 antibody or derivative or antigen-binding 20 fragment thereof. In some embodiments, the anti-PD-1 antibody selectively binds a PD-1 protein or fragment thereof. In some embodiments, the anti-PD1 antibody is nivolumab, pembrolizumab, or pidilizumab.

In some examples, the PD-L1 inhibitor is an anti-PDL-1 antibody or derivative or antigen-binding fragment thereof. In some examples, the anti-PD-L1 antibody or derivative or antigen-binding fragment thereof selectively binds a PD-L1 protein or fragment thereof. Examples of anti-PD-L1 antibodies and derivatives and fragments thereof are described in, e.g., WO 01/14556, WO 2007/005874, WO 2009/089149, WO 2011/066389, WO 2012/145493; U.S. Pat. Nos. 8,217,149, 8,779,108; US 2012/0039906, US 2013/0034559, US 2014/0044738, and US 2014/0356353. In some embodiments, the anti-PD-L1 antibody is MED14736 (durvalumab), MDPL3280A, 2.7A4, AMP-814, MDX-1105, atezolizumab (MPDL3280A), or BMS-936559.

In some examples, the anti-PD-L1 antibody is MEDI4736, also known as durvalumab. MEDI4736 is an anti-PD-L1 antibody that is selective for a PD-L1 polypeptide and blocks the binding of PD-L1 to the PD-1 and CD80 receptors. MEDI4736 can relieve PD-L1-mediated suppression of human T-cell activation in vitro and can further inhibit tumour growth in a xenograft model via a T-cell dependent mechanism. MEDI4736 is further described in, e.g., U.S. Pat. No. 8,779,108. The fragment crystallizable (Fc) domain of MED14736 contains a triple mutation in the constant domain of the IgG1 heavy chain that reduces binding to the complement component C1q and the Fey receptors responsible for mediating antibody-dependent cell-mediated cytotoxicity (ADCC).

CTLA4 inhibitors are inhibitors that block or reduce the activity of CTLA4. The immune checkpoint receptor cytotoxic T-lymphocyte associated antigen 4 (CTLA4 or CTLA-4) is expressed on T-cells and is involved in signaling pathways that reduce the level of T-cell activation. It is believed that CTLA4 can downregulate T-cell activation through competitive binding and sequestration of CD80 and CD86. In addition, CTLA4 has been shown to be involved in enhancing the immunosuppressive activity of T_{Reg} cells.

A CTLA4 inhibitor may prevent or reduce binding to CD80 and/or CD86. In some embodiments, a CTLA-4 inhibitor comprises an antibody binding compound, such as an antibody or an antigen-binding fragment thereof. U.S. Pat. Nos. 5,855,887; 5,811,097; 6,682,736; 7,452,535 disclose antibodies specific for human CTLA-4, including antibodies specific for the extracellular domain of CTLA-4 and which are capable of blocking its binding to CD80 or CD86; methods of making such antibodies, and methods of using such antibodies as anti-cancer agents. In some examples, the anti-CTLA-4 antibody is Tremelimumab, Ipilimumab, or Pembrolizumab.

TIGIT (T-cell immunoreceptor containing Ig and ITIM domains) belongs to the immunoglobulin superfamily, also known as Wucam, Vstm3 or Vsig9. TIGIT has an extracellular immunoglobulin domain, type I transmembrane domain and two Immune receptor tyrosine inhibition motif (ITIM). TIGIT is mainly distributed in regulatory T cells (Tregs), activated T cells and natural killer cells (NK), etc. It is a co-suppressive receptor protein, which can be combined with the positive proteins CD226 (Dnam-1) and APC on T cells. The expressed ligands CD155 (Pvr or Necl-5) and CD112 (Pvrl-2 or Nectin2) constitute a costimulatory network, Among them, TIGIT competes with CD226 to bind CD155 and CD112, and TIGIT binds its ligand with a higher affinity than CD226. The connection between TIGIT and CD155 or CD112 is mediated by its cytoplasmic ITIM or ITT-like motif, recruiting phosphatase SHIP-1 to the tail of TIGIT to trigger inhibitory signaling. In addition, the ITIM domain is also responsible for the inhibitory ability of mouse TIGIT.

Suitably, TIGIT inhibitors (such as anti-TIGIT antibodies) can inhibit, reduce, or neutralize one or more activities of TIGIT, for example, result in the blocking or reduction of immune checkpoints on T cells or NK cells, or The immune response is reactivated by adjusting antigen presenting cells. Examples of anti-TIGIT antibodies include Vibostolimab, Etigilimab, Tiragolumab, and Domvanalirnab.

The term “LAG-3” “LAG3”, or “Lymphocyte Activation Gene-3” refers to Lymphocyte Activation Gene-3. LAG-3's main ligand is MHC class II, to which it binds with higher affinity than CD4. The protein negatively regulates cellular proliferation, activation, and homeostasis of T cells, in a similar fashion to CTLA-4 and PD-land has been reported to play a role in Treg suppressive function. LAG3 is known to be involved in the maturation and activation of dendritic cells. A LAG-3 inhibitor can reduce or block the binding of LAG-3 to the MHC class II molecule, and thereby reduce or block its activity. Suitably, the LAG-3 inhibitor may be an anti-LAG-3 antibody, for example Favezelimab or Relatlimab.

TIM-3 is an immune checkpoint receptor that suppresses antitumor responses by negatively regulating the activity of CD8 T cells and antigen-presenting cells. A TIM-3 inhibitor may reduce or block the activity of TIM-3. Suitably, the TIM-3 inhibitor may be an anti-TIM-3 antibody, for example, Cobolimab.

B and T lymphocyte attenuator (BTLA) is an important co-signaling molecule. It belongs to the CD28 superfamily and is similar to programmed cell death-1 (PD-1) and cytotoxic T lymphocyte associated antigen-4 (CTLA-4) in terms of its structure and function. BTLA can be detected in most lymphocytes and induces immunosuppression by inhibiting B and T cell activation and proliferation. BTLA is found to be expressed in tumor-infiltrating lymphocytes (TILs) and is often associated with impaired anti-tumor immune response. A BTLA inhibitor may reduce or block the activity of BTLA. Such a reduction or blockage may increase B and T cell activation and proliferation. Suitably, the BTLA inhibitor may be an anti-BTLA antibody, for example, Tifcemalimab.

Killer immunoglobulin-like receptors (KIRs), are a family of cell surface proteins found on natural killer (NK) cells. They inhibit the killing function of these cells by interacting with MHC class I molecules. KIR inhibitors may reduce or block the activity of KIR. Such a reduction or blockage may increase the killing ability of NK cells. Suitably, a KIR inhibitor may be an anti-KIR antibody, for example, Lirilumab.

Suitably, the immune checkpoint inhibitor may be selected from the group consisting of a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and KIR inhibitor.

Suitably, the immune checkpoint inhibitor may be an antibody. For example, the immune checkpoint inhibitor may be an anti-PD-1 antibody, an anti-PD-L1 antibody, an anti-PD-L2 antibody, an anti-CTLA4 antibody, an anti-TIGIT antibody, an anti-LAG-3 antibody, an anti-TIM-3 antibody, an anti-BTLA antibody, and/or anti-KIR antibody.

Monoclonal antibodies, antibody fragments, and antibody derivatives for blocking immune checkpoint pathways can be prepared by any of several methods known to those of ordinary skill in the art, including but not limited to, somatic cell hybridization techniques and hybridoma, methods. Hybridoma generation is described in Antibodies, A Laboratory Manual, Harlow and Lane, 1988, Cold Spring Harbor Publications, New York. Human monoclonal antibodies can be identified and isolated by screening phage display libraries of human immunoglobulin genes by methods described for example in U.S. Pat. Nos. 5,223,409, 5,403,484, 5,571,698, 6,582,915, and 6,593,081. Monoclonal antibodies can be prepared using the general methods described in U.S. Pat. No. 6,331,415 (Cabilly).

As an example, human monoclonal antibodies can be prepared using a XenoMouse™ (Abgenix, Freemont, CA) or hybridomas of B cells from a XenoMouse. A XenoMouse is a murine host having functional human immunoglobulin genes as described in U.S. Pat. No. 6,162,963 (Kucherlapati).

Methods for the preparation and use of immune checkpoint antibodies are well known in the art, and merely by way of example, some are described in the following illustrative publications. The preparation and therapeutic uses of anti-CTLA-4 antibodies are described in U.S. Pat. No. 7,229,628 (Allison), 7311910 (Linsley), and 8017144 (Korman). The preparation and therapeutic uses of anti-PD-1 antibodies are described in U.S. Pat. No. 8,008,449 (Korman) and U.S. Patent Application No. 2011/0271358 (Freeman). The preparation and therapeutic uses of anti-PD-L1 antibodies are described in U.S. Pat. No. 7,943,743 (Korman). The preparation and therapeutic uses of anti-TIM-3 antibodies are described in U.S. Pat. No. 8,101,176 (Kuchroo) and 8552156 (Tagayanagi). The preparation and therapeutic uses of anti-LAG-3 antibodies are described in U.S. Patent Application No. 2011/0150892 (Thudium) and International Publication Number WO2014/008218 (Lonberg). The preparation and therapeutic uses of anti-KIR antibodies are described in U.S. Pat. No. 8,119,775 (Moretta). The preparation of antibodies that block BTLA regulated inhibitory pathways (anti-BTLA antibodies) are described in U.S. Pat. No. 8,563,694 (Mataraza). In particular examples, the inhibitor of PD1 and/or PD-L1 may be as described in U.S. Pat. No. 8,354,509B2 and U.S. Pat. No. 8,900,587B2 which are incorporated herein by reference. For example, the immune checkpoint therapy is pembrolizumab (also known as KEYTRUDA).

The immune checkpoint inhibitor can be administered in an amount and for a time (e.g., for a particular therapeutic regimen over time) to provide an improvement of the pathology and/or symptoms associated with the cancer or pre-cancer as described herein above.

The immune checkpoint inhibitor may be formulated, dosed, and administered in a fashion consistent with good medical practice. Factors for consideration in this context include, the particular subject being treated, the clinical condition of the individual patient, the cause of the disorder, the site of delivery of the agent, the method of administration, the scheduling of administration, and other factors known to medical practitioners. A “therapeutically effective amount” of an immune checkpoint inhibitor to be administered will be governed by such considerations, and is the minimum amount necessary to prevent, ameliorate, or treat, or stabilize, a benign, precancerous, or early stage cancer; or to treat or prevent the occurrence or recurrence of a tumour, a dormant tumour, or a micrometastases, for example, when used as a neoadjuvant. The immune checkpoint inhibitor need not be, but is optionally, formulated with one or more agents currently used to prevent or treat cancer.

Suitable routes of administration of an immune checkpoint inhibitor include, without limitation, oral, parenteral, subcutaneous, rectal, transmucosal, intestinal administration, intramuscular, intramedullary, intrathecal, direct intraventricular, intravenous, intravitreal, intraperitoneal, intranasal, or intraocular injections. Alternatively, one may administer an immune checkpoint inhibitor in a local rather than systemic manner, for example, via injection of an immune checkpoint inhibitor directly into a solid tumour, or by topical application (for example to a skin cancer).

An immune checkpoint inhibitor may be formulated according to known methods to prepare pharmaceutically useful compositions, whereby the inhibitor is combined in a mixture with a pharmaceutically suitable excipient or carrier. Sterile phosphate-buffered saline is one example of a pharmaceutically suitable excipient. Other suitable excipients are well-known to those in the art. See, for example, Ansel et al, PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

Generally, the dosage of an administered immune checkpoint inhibitor for humans will vary depending upon such factors as the patient's age, weight, height, sex, general medical condition and previous medical history. It may be desirable to provide the subject with a dosage that is in the range of from about 1 mg/kg to 24 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m2 for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed.

Suitably, the immune checkpoint inhibitor may be employed in the use or method as described herein as a sole treatment for cancer or pre-cancer, or in conjunction with a second treatment for cancer or pre-cancer, such as a surgery, radiation, chemotherapy, immunotherapy, hormone therapy, vaccine treatment, or any combination thereof.

Suitably, the immune checkpoint inhibitor may be employed as first, second, third, or further, line treatment for cancer or pre-precancer.

In some aspects, the invention relates to an agent that alters the redox status (for example alters the lactate to glucose ratio) in a cancer or a pre-cancer and it uses in sensitising a subject (their cancer or pre-cancer) to a immune checkpoint inhibitor (for example PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and/or KIR inhibitor). For example, the agent alters the lactate to glucose ratio in the interstitial fluid of the cancer or pre-cancer.

In order for the agent to alter the redox status (for example alters the lactate to glucose ratio), the cells of the cancer or pre-cancer must be exposed to the agent. In this context, the term “expose” refers to an active step of contacting the cancer or pre-cancer cells with the agent so as to alter the redox status (for example lactate to glucose ratio) and/or providing to a cancer or pre-cancer cell an agent that alters redox status (for example the lactate to glucose ratio). Exposure may be in vitro, in vivo or ex vivo. Upon exposure in vitro or ex vivo, the cells may be introduced (e.g. re-introduced) into the subject with cancer or pre-cancer.

The term “redox status” or “metabolic status” as used herein refers to the cytosolic and/or mitochondrial ratio of NAD+:NADH in the cancer or pre-cancer microenvironment, such as the tumour as a whole and/or interstitial fluid of the cancer or pre-cancer (also referred to herein as the interstitial fluid of the tumour). The inventors have found that both decreasing the NAD+:NADH ratio (mtDNA mutation) and/or increasing the NAD+:NADH ratio away from homeostatic levels within cancer and/or pre-cancer cells exerts an immunomodulatory effect on tumours, rendering these more sensitive to immune checkpoint inhibitors. Homeostatic levels in this context may refer to the levels in wild-type (for example non-cancerous cells, and/or cancer cells that do not bear mtDNA mutations). NAD+:NADH ratio is tightly regulated in cells—as the directionality and activity of a huge number of reactions (glycolysis, gluconeogenesis, fatty acid synthesis, DNA repair (PARP is NAD+ dependent) histone acetylation etc) are dependent on it.

In some embodiments, the agent may be provided by transducing or transfecting the cells of the cancer or pre-cancer with a nucleic acid encoding an agent that alters the redox status (for example alters the lactate to glucose ratio). Suitably, the encoded agent may be an enzyme. Suitably, the enzyme may be an enzyme that increases glucose uptake and/or lactate release.

For example, the enzyme may be selected from the group consisting of NADH oxidase and NADPH oxidase. By way of example, the NADH oxidase may be from Lactobacillus brevis. Such an enzyme may be referred to herein as “LbNOX”. The enzyme may suitably be expressed in the cytosol of the cancer or pre-cancer cells. LbNOX expressed in the cytosol may be referred to herein as cytoLbNOX. Alternatively, or additionally, the enzyme may suitably be expressed in the mitochondria of the cancer or pre-cancer cells. LbNOX expressed in the mitochondria may be referred to herein as mitoLbNOX.

By comparing bulk tumour metabolite changes of B78-D14 m.12,436^{80%}, Hcmel12 m.12,436^{80%}, and Hcmel12 cytoLbNOX tumours, which did not show any changes in common metabolites, the inventors believe that, surprisingly, an altered redox status (for example altered cellular redox state of the cancer and/or pre-cancer) irrespective of direction, and not gross metabolite abundance change is sufficient to alter the tumour immune microenvironment and sensitise the cancer or pre-cancer to a treatment with an immune checkpoint inhibitor. In this context, “irrespective of direction” may refer to the changes in direction of the NAD+:NADH ratios.

Suitably, the agent (such as an NADH oxidase, for example cytoLbNOX and/or mitoLbNOX) may be for use in combination with a tumour-associated neutrophil reducing compound. A tumour-associated neutrophil reducing compound is a compound that decreases the number of tumour resident neutrophils within a tumour. Herein tumour resident neutrophils may also be referred to as tumour-associated neutrophils. The reduction may be, for example, by blocking tumour resident neutrophil infiltration into the tumour, by reducing the number of neutrophils in the subject (for example by killing and/or blocking the production/maturation of neutrophils), or both. Killing of the neutrophils may be by antibody-dependent cell-mediated cytotoxicity (ADCC). Compounds that may reduce tumour resident neutrophils include for example anti-Ly6G antibody, anti-GR1 antibody, and/or other antibodies that are specific to certain neutrophil antigens (such as antibodies that are specific to the human neutrophil antigens (HNAs), selected from the group consisting of HNA-1a, HNA-1b, and HNA-1c). These antibodies can be used to identify and deplete neutrophils that express these antigens.

The term “altered” as used herein refers to a change, which may be an increase or a decrease, relative to a reference value.

Suitably, the agent described herein alters the NAD+:NADH ratio in a cancer or a pre-cancer. As mentioned elsewhere herein, the alteration may be an increase or a decrease in the NAD+:NADH ratio.

Suitably, the agent described herein increases the lactate to glucose ratio in a cancer or a pre-cancer. Suitably, the agent increases the lactate to glucose ratio in the tumour to above 2.5:1, 3:1, 3.5:1, 4:1 or more.

Suitably, the agent described herein increases the lactate to glucose ratio in the interstitial fluid of a cancer or a pre-cancer. Suitably, the agent increases the lactate to glucose ratio in the interstitial fluid of the tumour to above 2.5:1, 3:1, 3.5:1, 4:1 or more.

The term “increased” or “increase” as used herein generally means a difference between the relevant level (metabolite, mutation load etc) and a suitable corresponding reference value, that is at least about 10% greater than the reference value, for example at least about 20%, at least about 30%, at least about 40%, at least about 50%, at least about 60%, at least about 70%, at least about 80%, at least about 90% greater than the reference value.

The term “decrease” or “decreased” as used herein, generally means a difference between the relevant level (metabolite, mutation load etc) and a suitable corresponding reference value that is at a reduction of least about 5%, at least about 10%, at least about 20%, at least about 30%, at least about 40%, at least about 50%, at least about 60%, at least about 70%, at least about 80%, at least about 90% etc than the reference value.

In the context of an agent that alters the lactate to glucose ratio, as used herein, the “reference value” may be the corresponding parameter (such as an NAD+:NADH ratio, or an lactate to glucose ratio) of a cancer or a pre-cancer prior to the cancer or pre-cancer being exposed to the agent. Many agents that alter redox status (for example alter the lactate to glucose ratio) of a cancer or a pre-cancer are known in the art. Additionally, methods of determining the level of lactate and glucose are known in the art and may be used as a matter of routine (see for example Cengiz et al. 2009 doi: 10.1089/dia.2009.0002; and Spahar-Deleze et al. 2021 doi: 10.3390/chemosensors9080195). Assays for measuring NAD+:NADH ratio are also widely known in the art.

The agent may be used as a pre-treatment. In this context, the agent may be considered as a neoadjuvant. The agent may be provided prior to, or simultaneously with, the immune checkpoint inhibitor (such as PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and/or KIR inhibitor).

The agent may be formulated as appropriate. For example, the agent may be an infusion. As used herein, “infusion” refers to a solution, emulsion or suspension. In one example, the agent may be injected into the cancer or pre-cancer. Typically, the agent is agent is a cell permeable compound or a pre-cursor thereof.

The agent may be in the form of a pharmaceutical composition. The pharmaceutical composition may further comprise a pharmaceutically acceptable diluent, carrier or excipient. Such compositions may further routinely contain pharmaceutically acceptable concentrations of salt, buffering agents, preservatives, compatible carriers, supplementary immune potentiating agents such as adjuvants and cytokines and optionally other therapeutic agents.

The compositions may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

It will be appreciated that the pharmaceutical compositions described above may be suitable for use in treating cancer or precancer, and particularly those various forms of cancer described herein.

The agent may be for administration to the subject by any suitable route by which a therapeutically effective amount of the agent may be provided.

The agent may be any suitable agent for example, it may be a small molecule, a metabolite, an antibody, a nucleic acid, an enzyme etc.

Suitably, the enzyme may be an NADH oxidase, for example from Lactobacillus brevis. Such an enzyme may be referred to herein as “LbNOX”. The enzyme may suitably be expressed in the cytosol of the cancer or pre-cancer cells.

Suitably, the nucleic acid may encode an NADH oxidase, for example from Lactobacillus brevis (i.e. LbNOX). The nucleic acid may be incorporated into a distinct nucleic acid sequence, such as a vector.

In one example, the vector is a plasmid, a viral vector, or a cosmid, optionally wherein the vector is selected from the group consisting of a lentivirus, retrovirus, adeno-associated virus, adenovirus, vaccinia virus, canary poxvirus, herpes virus, minicircle vector and synthetic DNA or RNA.

As used herein, the term “vector” refers to a nucleic acid sequence capable of transporting another nucleic acid sequence to which it has been operably linked. The vector can be capable of autonomous replication or it can integrate into a host DNA. The vector may include restriction enzyme sites for insertion of recombinant DNA and may include one or more selectable markers or suicide genes. The vector can be a nucleic acid sequence in the form of a plasmid, a bacteriophage or a cosmid. Preferably the vector is suitable for expression in a cell (i.e. the vector is an “expression vector”). Preferably, the vector is suitable for expression in a human T cell such as a CD8⁺ T cell or CD4⁺ T cell, or stem cell, iPS cell, or NK cell. In certain aspects, the vector is a viral vector, such as a retroviral vector, a lentiviral vector or an adeno-associated vector. Optionally, the vector is selected from the group consisting of an adenovirus, vaccinia virus, canary poxvirus, herpes virus, minicircle vector and synthetic DNA or synthetic RNA.

Preferably the (expression) vector is capable of propagation in a host cell and is stably transmitted to future generations.

The vector may comprise regulatory sequences. “Regulatory sequences” as used herein, refers to, DNA or RNA elements that are capable of controlling gene expression. Examples of expression control sequences include promoters, enhancers, silencers, TATA—boxes, internal ribosomal entry sites (IRES), attachment sites for transcription factors, transcriptional terminators, polyadenylation sites etc. Optionally, the vector includes one or more regulatory sequences operatively linked to the nucleic acid sequence to be expressed. Regulatory sequences include those which direct constitutive expression, as well as tissue-specific regulatory and/or inducible sequences.

Optionally, the vector comprises the nucleic acid sequence of interest operably linked to a promoter. “Promoter”, as used herein, refers to the nucleotide sequences in DNA to which RNA polymerase binds to start transcription. The promoter may be inducible or constitutively expressed. Alternatively, the promoter is under the control of a repressor or stimulatory protein. The promoter may be one that is not naturally found in the host cell (e.g. it may be an exogenous promoter). The skilled person in the art is well aware of appropriate promoters for use in the expression of target proteins, wherein the selected promoter will depend on the host cell.

“Operably linked” refers to a single or a combination of the below-described control elements together with a coding sequence in a functional relationship with one another, for example, in a linked relationship so as to direct expression of the coding sequence.

The vector may comprise a transcriptional terminator. “Transcriptional terminator” as used herein, refers to a DNA element, which terminates the function of RNA polymerases responsible for transcribing DNA into RNA. Preferred transcriptional terminators are characterized by a run of T residues preceded by a GC rich dyad symmetrical region.

The vector may comprise a translational control element. “Translational control element”, as used herein, refers to DNA or RNA elements that control the translation of mRNA. Preferred translational control elements are ribosome binding sites. Preferably, the translational control element is from a homologous system as the promoter, for example a promoter and its associated ribozyme binding site. Preferred ribosome binding sites are known, and will depend on the chosen host cell.

The vector may comprise restriction enzyme recognition sites. “Restriction enzyme recognition site” as used herein, refers to a motif on the DNA recognized by a restriction enzyme.

The vector may comprise a selectable marker. “Selectable marker” as used herein, refers to proteins that, when expressed in a host cell, confer a phenotype onto the cell which allows selection of the cell expressing said selectable marker gene. Generally this may be a protein that confers a new beneficial property onto the host cell (e.g. antibiotic resistance) or a protein that is expressed on the cell surface and thus accessible for antibody binding. Appropriate selectable markers are well known in the art.

Optionally, the vector may also comprise a suicide gene. “Suicide gene” as used herein, encodes a protein that induce death of the modified cell upon treatment with specific drugs. By way of example, suicide can be induced in cells modified by the herpes simplex virus thymidine kinase gene upon treatment with specific nucleoside analogs including ganciclovir, cells modified by human CD20 upon treatment with anti-CD20 monoclonal antibody and cells modified with inducible Caspase9 (iCasp9) upon treatment with AP1903 (reviewed by BS Jones, LS Lamb, F Goldman, A Di Stasi; Improving the safety of cell therapy products by suicide gene transfer. Front Pharmacol. (2014) 5:254). Appropriate suicide genes are well known in the art.

Preferably the vector comprises those genetic elements which are necessary for expression of the binding proteins described herein by a host cell. The elements required for transcription and translation in the host cell include a promoter, a coding region for the protein(s) of interest, and a transcriptional terminator.

A person of skill in the art will be well aware of the molecular techniques available for the preparation of (expression) vectors and how the (expression) vectors may be transduced or transfected into an appropriate host cell (thereby generating a modified cell described further below). The (expression) vector system described herein can be introduced into cells by conventional techniques such as transformation, transfection or transduction. “Transformation”, “transfection” and “transduction” refer generally to techniques for introducing foreign (exogenous) nucleic acid sequences into a host cell, and therefore encompass methods such as electroporation, microinjection, gene gun delivery, transduction with retroviral, lentiviral or adeno-associated vectors, lipofection, superfection etc. The specific method used typically depends on both the type of vector and the cell. Appropriate methods for introducing nucleic acid sequences and vectors into host cells such as human cells are well known in the art; see for example Sambrook et al (1989) Molecular Cloning, A Laboratory Manual, Cold Spring Harbor Laboratory, Cold Spring Harbor, N.Y; Ausubel et al (1987) Current Protocols in Molecular Biology, John Wiley and Sons, Inc., NY; Cohen et al (1972) Proc. Natl. Acad. Sci. USA 69, 2110; Luchansky et al (1988) Mol. Microbiol. 2, 637-646.

Suitable examples of agents that alter the redox status (for example lactate to glucose ratio) in a cancer or a pre-cancer are provided below. As would be clear to a person of skill in the art, these agents may be used to alter the redox status (for example lactate to glucose ratio) 25 in the interstitial fluid of a cancer or a pre-cancer.

Suitably, the agent may be a compound that drives glycolytic flux through MDH1. For example, the agent may be isocitrate, aconitate, citrate, oxaloacetate, or a NADH or NAD+ precursor.

Suitably, the agent may be a compound that modulates NAD(H) redox handling via the malate-aspartate shuttle. For example, the compound may be selected from the group consisting of: isocitrate, aconitate, citrate, oxaloacetate, malate, fumarate, argininosuccinate.

Suitably, the agent may be lactate. In one example, the lactate may be administered to the cancer or pre-cancer as a lactate infusion.

Suitably, the agent may be a glucose metabolising enzyme and/or a lactate metabolising enzyme. Optionally, the glucose metabolising enzyme may be selected from the group consisting of hexokinase, phosphorglucoisomerase, phosphofructokinase, aldolase, isomerase, triose-phosphate isomerase, glyceraldehyde-3-phosphatedehydrogenase, phosphoglycerate kinase, phosphoglycerate mutase, enolase, pyruvate kinase. Optionally, the lactate metabolising enzyme is lactate dehydrogenase (LDH) (e.g. lactate dehydrogenase A and/or lactate dehydrogenase B).

Suitably, the agent may be an inhibitor of an enzyme that decreases glycolytic flux in cancer cells or pre-cancer cells. Optionally the enzyme may be pyruvate dehydrogenase or pyruvate carboxylase.

Suitably, the agent may be an activator of an enzyme that increases lactate efflux in cancer cells or pre-cancer cells. Optionally, the enzyme is MDH1 or GAPDH.

Suitably, the agent may be a small molecule inhibitor of an enzyme in the malate-aspartate shuttle. Optionally, the enzyme is selected from the group consisting ofGOT1, GOT2, MDH1, MDH2, Glutamate-Aspartate carrier, and a-ketoglutarate-malate carrier.

Suitably, the agent may be a small molecule activator of an enzyme in the malate-aspartate shuttle. Optionally, the enzyme may be selected from the group consisting of GOT1, GOT2, MDH1, MDH2, Glutamate-Aspartate carrier, and a-ketoglutarate-malate carrier.

Suitably, the agent may be an inhibitor of complex I, complex II, complex III or complex IV. Suitably, an inhibitor of Complex I inhibitor may be rotenone. Suitably, an inhibitor of Complex II inhibitor may be thenoyltrifluoroacetone. Suitably, an inhibitor of Complex III inhibitor may be selected from the group consisting of antymycim A, Myxothiazol, and Stigmatellin. Suitably, an inhibitor of Complex IV inhibitor may be cyanide.

Suitably, the agent that alters redox status may alter the pyruvate to lactate ratio. Thus, an altered redox status may be indicated by an altered pyruvate to lactate ratio.

The inventors established a link between an altered immune cell population within the tumour microenvironment, an altered metabolic status in the cancer or pre-cancer, and a high deleterious mtDNA mutation load. On the basis of this, the inventors believe that increasing the deleterious mtDNA mutation load in a cancer or pre-cancer will sensitise the cancer or pre-cancer to a treatment with an immune checkpoint inhibitor.

Accordingly, the agent that alters the redox status, for example an agent that alters (e.g. increases) the lactate to glucose ratio, in a cancer or a pre-cancer may be a compound that increases a deleterious mtDNA mutation load in the cancer or pre-cancer. A compound that increases a deleterious mtDNA mutation load may do so by either mutating individual mtDNA molecules or by removing unmutated mtDNA molecules. Methods of determining the deleterious mitochondrial DNA (mtDNA) mutation load in a cancer or pre-cancer sample from the subject are known in the art.

Optionally, the compound induces a deleterious mtDNA mutation (i.e. introduces a mutation into the mtDNA of the cancer or pre-cancer).

Suitably, the agent may be a compound that increases a deleterious mtDNA mutation load in the cancer or pre-cancer, wherein the compound is selected from the group consisting of a mitochondrial base editing enzyme (such as DdCBEs) and a mitochondrial heteroplasmy manipulating enzyme (such as mtZFNs, mitoTALENs, or other nucleases). It will be appreciated that the increase in this context is compared to the mutation load prior to the cancer or pre-cancer being exposed to the agent as described herein.

The term “deleterious mtDNA mutation” as used herein refers to a mutation that adversely affects the structure and/or function of the mtDNA element it encodes, in contrast to a neutral mutation (such as a silent point mutation), which has neither a positive or negative mutation on the corresponding encoded element.

Methods of identifying deleterious mtDNA mutations will be known in the art. Merely by way of example, a deleterious mtDNA mutation may be selected from the group consisting of:
(i) a tRNA mutation having a MitoTIP RAW score of at least 12.6, or at least 16.25;
(ii) a rRNA mutation;
(iii) a truncation mutation in a mtDNA gene;
(iv) a missense mutation in a mtDNA gene, wherein the missense mutation has an Apogee score of more than 0.5, optionally wherein the missense mutation is selected from a frameshift mutation, an insertion mutation or a deletion mutation; and/or
(v) a mutation in a mtDNA D-loop region selected from the group consisting of: the H-strand promoter (m. 545-567), hypervariable segment 2 (MT-HV2; m.57-372), and hypervariable segment 1 (MT-HV1; m.16024-16390).

The tRNA mutation may be in a gene selected from the group consisting of MT-TL1, MT-TA, MT-TC, MT-TD, MT-TE, MT-TF, MT-TG, MT-TH, MT-TI, MT-TK, MT-TL2, MT-TM, MT-TN, MT-TP, MT-TQ, MT-TR, MT-TS1, MT-TS2, MT-TT, MT-TV, MT-TW, and MT-TY.

The rRNA mutation may be in a gene selected from the group of MT-RNR1 and MT-RNR2.

The truncation or missense mutation may be in a tRNA, rRNA or protein coding gene.

The protein coding gene may be selected from the group of MT-ND5, MT-ND1, MT-ND2, MT-ND3, MT-ND4, MT-ND4L, MT-ND6, MT-CO1, MT-C02, MT-C03, MT-CYB, MT-ATP6, and MT-ATP8. These genes encode proteins that are subunits of the mitochondrial respiratory chain complexes, specifically NADH: ubiquinone oxidoreductase (complex I), ubiquinol:cytochrome c oxidoreductase (complex III), cytochrome c oxidase (complex IV), or ATP synthase (complex V). Accordingly, the mutation may be in a mtDNA gene that encodes a subunit of a mitochondrial respiratory chain complex selected from the group consisting of complex I, complex III, complex IV and complex V.

Suitably, the deleterious mtDNA mutation is a truncation, missense, insertion, or frameshift mutation.

Suitably the deleterious mutation may be in the gene MT-ND5. Suitably, the deleterious mutation may be a truncating mutation that is in a region selected from: m.12418-12425:A indel or m.12385-12390:C indel.

Suitably, the deleterious mutation may be a missense mutation in the MT-CO1, MT-ND5, MT-ND4, MT-CYB or MT-TY gene.

Suitably, the missense mutation may be selected from the group consisting of m.6318C>T, m.12730G>A, m.11736T>C, m.15140G>A, m.5843A>G, and m.6214G>A.

Suitably, the insertion mutation may be selected from the group consisting of m.16183:CC indel, and m.16192:T indel.

The terms “mtDNA mutation load”, “mtDNA heteroplasmy”, “variant allele frequency”, or “VAF” refer to mtDNA mutations that arise and co-exist with the wild-type allele in the same cell, or group of cells. In the context of the present disclosure, the term “determine” or “determining” refers to measuring the level of mtDNA molecules comprising a deleterious mutation in a cell or group of cells and comparing that level to the level of mtDNA molecules that do not comprise such deleterious mutations (or to the total number of mtDNA molecules that are present in the cell or group of cells). It will be appreciated that mtDNA molecules that do not comprise deleterious mutations may comprise other mutations, however these mutations would not be deleterious within the meaning of the present disclosure.

MtDNA mutation load may be typically represented as a percentage. For example a mutation load of 30% means that 30% of mtDNA molecules in a cell or group of cells (such as a sample) carry a deleterious mtDNA mutation. The deleterious mutation may be the same or different in all mutated mtDNA molecules. More suitably, the deleterious mutation may be the same in all mutated mtDNA molecules for the purpose of measuring mutation load. However, it will be appreciated that the mtDNA molecules with the deleterious mutation used for determining mutation load may have further (additional) deleterious mutations.

Methods of determining mtDNA mutation loads are well known in the art and include mtDNA sequencing, such as single cell mtDNA sequencing. Methods of determining mtDNA mutation loads are described in, for example, Sobenin et al, 2014 (doi: 10.1155/2014/292017).

In the context of some of the methods disclosed herein, the deleterious mtDNA mutation load is determined in a cancer or pre-cancer sample from the subject.

The term “sample” refers to any group of cells comprising cancer cells and/or pre-cancer cells derived from the subject. The sample may typically comprise a mixture of healthy (i.e. non-cancerous and non-precancerous cells) and cancer cells (and/or pre-cancerous cells). The sample may comprise components of a tumour e.g. cells (cancer, pre-cancer, and healthy cells), as well as interstitial fluid.

Suitably, the sample will comprise at least 5%, at least 10%, at least 15%, at least 20%, or more of cancer and/or pre-cancer cells. For example the sample may comprise at least 25%, at least 30%, at least 35%, at least 40%, at least 45%, at least 50% or more of cancer and/or pre-cancer cells.

The presence of healthy cells, which may be substantially free of a deleterious mtDNA mutation load, may lower the determined (overall) deleterious mtDNA mutation load in a sample as compared to if the deleterious mtDNA mutation load was determined solely or substantially only on cancer or pre-cancer cells.

In this context the term “substantially only” means that the cancer cells account for at least 50%, at least 60%, at least 70%, at least 80%, at least 90%, or more of the cells in the sample.

Thus, merely by way of example, when a deleterious mtDNA mutation load in a cancer or pre-cancer sample obtained from a subject is determined to be about 30%, the deleterious mtDNA mutation load of the cancer or precancer cells present in the sample specifically may be more than 30%, more than 40%, more than 50%, more than 60%, more than 70%, more than 80%, or more than 90%.

Suitably, with reference to a solid cancer, the sample may be a biopsy, a smear sample, or a interstitial fluid sample.

Suitably, with reference to a liquid cancer, the sample may be a blood sample (for example, a whole blood sample, a blood plasma sample, or a serum sample), or a urine sample.

As will be clear to the person skilled in the art, the amount of mtDNA molecules having the deleterious mutation is used to determine the level of the mutation load in a cell or a group of cells. Herein, the proportion of mtDNA molecules having the deleterious mutation is referred to as “the deleterious mtDNA mutation load”. The skilled person would appreciate that a low proportion of mtDNA molecules having the deleterious mutation will correspond to a low deleterious mtDNA mutation load, which may be asymptomatic (i.e. have little or no impact on overall mitochondrial function of the cell). Conversely, a high proportion of mtDNA molecules having the deleterious mutation will correspond to a high deleterious mtDNA mutation load, which in the context of the present disclosure may be symptomatic, i.e. have an adverse effect on overall mitochondrial function of the cell. An adverse effect on overall mitochondrial function of the cell may be determined by an altered redox status (for example mitochondrial and/or cytosolic metabolic state), which may be due to: altered mitochondrial redox homeostasis, reduced oxidative phosphorylation, increased oxidative stress, or any combination thereof.

These changes may further lead to alterations in the cancer or pre-cancer microenvironment, such as the tumour as a whole and/or interstitial fluid of the cancer or pre-cancer (also referred to herein as the interstitial fluid of the tumour). The altered tumour microenvironment may be more or less favourable for specific immune cell populations, as explained in more detail hereinbelow.

An altered redox status may be indicated by an increase in one or more cellular metabolite selected from the group consisting of: fumarate, lactate, malate, acetyl CoA, aspartate, glucose, glucose 6-phosphate, glutamine, glucose 3-phosphate, glycolytic intermediates, fumarate adducts (such as succinicGSH and/or succinylCysteine), and arginosuccinate. In particular, altered redox status may be indicated by increase in the fumarate adducts succinicGSH and/or succinylCysteine (also referred to as succ.cys and succ.gsh respectively herein).

Additionally, or alternatively, altered redox status may include a decrease in one or more cellular metabolite selected from the group consisting of: alpha-ketoglutarate, pyruvate, phosphoenolpyruvate and succinate.

A deleterious mtDNA mutation load may alter the NAD+:NADH ratio in the mitochondria and/or the cytosol. Suitably, the deleterious mtDNA mutation load may increase the NAD+:NADH ratio in the mitochondria and/or the cytosol. As shown in the examples, disturbed NAD+:NADH ratio may result in partial reverse flux of MDH2 within mitochondria (which can be determined from the ratio of pyruvate carboxylase-derived (m+3) malate, citrate, and aconitate, and pyruvate).

Merely by way of example, altered redox status may include changes in TCA cycle and/or urea cycle metabolites. Suitably, these metabolites may be related to the malate-aspartate shuttle (MAS) and fumarate within mitochondria and/or within the cytosol. As described in detail in the examples, the inventors used 1-¹³C-glutamine tracing, which revealed that NAD+:NADH ratio changes are associated with increases in malate m+1 abundance, and argininosuccinate m+1 abundance, but not a-KG m+1, aconitate m+1 or aspartate m+1-implicating increased MDH1 flux. Accordingly, altered mitochondrial metabolic state may include increased MDH1 flux.

Merely by way of example, altered redox status may include an imbalance between lactate and glucose in the tumour (e.g. in the interstitial fluid of the tumour). As described elsewhere herein an altered lactate to glucose ratio in a cancer or pre-cancer can sensitise the cancer or pre-cancer to a PD-1 inhibitor and/or PD-L1 inhibitor. In this context, and altered lactate to glucose ratio may be an increased lactate to glucose ratio.

Merely by way of example, altered redox status may include an imbalance between pyruvate and lactate in the tumour (e.g. in the interstitial fluid of the tumour). Accordingly, a deleterious mtDNA mutation load may alter the redox status (for example alter the pyruvate to lactate ratio) in the tumour (e.g. in the interstitial fluid of the tumour).

The inventors believe that an altered redox status (for example altered lactate to glucose ratio) in the cancer or pre-cancer is the reason why such cancers or pre-cancers have a notably different proportion of immune cells in the tumour microenvironment. In view of the data in the Examples section herein, the inventors believe that an altered lactate to glucose ratio in the cancer or pre-cancer is associated with increased levels of immune cells selected from the group consisting of: NK cells; monocytes; CD4+ T cells; and ISG-expressing immune cells, and/or decreased levels of macrophages (for example tumour associated macrophages) and/or neutrophils.

Suitably, in the context of the present disclosure, the agent may be an agent that increases levels of immune cells selected from the group consisting of: NK cells; monocytes; CD4+ T cells; and ISG-expressing immune cells, and/or decreased levels of macrophages (for example tumour associated macrophages) and/or neutrophils.

Suitably the agent may decreases levels of neutrophils, for example tumour infiltrating neutrophils. It will be appreciated that such an agent may decrease the levels of neutrophils (for example tumour infiltrating neutrophils) by altering the redox status in a cancer or a pre-cancer.

Accordingly, in further aspects the present invention provides an immune checkpoint inhibitor for use in treating a subject having a cancer or a pre-cancer, wherein the subject has been exposed to an agent that reduces neutrophils (for example tumour infiltrating neutrophils).

The present invention also provides a method of sensitising a subject having a cancer or a pre-cancer to an immune checkpoint inhibitor, comprising exposing the subject to an agent that that reduces neutrophils (for example tumour infiltrating neutrophils).

The present invention also provides a method of treating a cancer or a pre-cancer in a subject, comprising administering an immune checkpoint inhibitor to the subject, wherein the subject has been exposed to an agent that reduces neutrophils (for example tumour infiltrating neutrophils).

The present invention also provides a method of treating a cancer or a pre-cancer in a subject, comprising:
(i) exposing the subject to an agent that that reduces neutrophils (for example tumour infiltrating neutrophils); and
(ii) administering an immune checkpoint inhibitor to the subject.

The term “NK cells” or “Natural Killer cells” as used herein refers to a subset of peripheral blood lymphocytes defined by the expression of CD56 or CD16 and the absence of the T cell receptor (CD3).

The term “monocytes” as used herein refers to a subset of immune cells that are produced in the bone marrow and migrate through the blood to tissues in the body, where they become a macrophage. Suitably the monocytes are immature, intermediate or classical monocytes. Immature monocytes are Lys6C and F480 positive. Intermediate monocytes are CD14+ and CD16+. Classical monocytes are CD14+ and CD16−.

The term “CD4 NK-like T cells” as used herein refers to a subset of immune cells that are cytotoxic T-cells that co-express NK receptors such as CD56, CD16, and/or CD57.

The term “CD4+ T cells” refers to T helper cells.

The term “ISG-expressing immune cells” refers to a subset of cells that express interferon-stimulated genes.

The term “macrophages” refers to a subgroup of phagocytic cells produced by monocyte differentiation. The term “tumour associated macrophages” (TAMs) generally refers to macrophages that exist in the microenvironment of a cancer, for example, a tumour.

The term “neutrophil” refers to a type of granulocytes of white blood cells which are first-responders of inflammatory cells. In come cancers and/or precancers, neutrophils may be present within the tumour. Such neutrophils may be referred to as tumour infiltrating neutrophil (TANs). The presence of TANs may be associated with poor prognosis.

Suitably, NK cell levels may be increased by at least 100%, at least 150%, at least 200% etc. Suitably, the levels of tumour associated macrophages may be decreased by at least 25%, at least 50%, at least 75% etc. Suitably, the levels of immature monocytes may be increased by at least 100%, at least 150%, at least 200% etc.

Suitably, CD4+ T cell levels may be increased by at least 20%, at least 50%, at least 100%, at least 200%, etc.

Suitably, neutrophil levels may be decreased by at least 20%, at least 30%, at least 40%, at least 50%, at least 60%, at least 70%, at least 80%, at least 90%, or more.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1

### Results

The inventors have performed the experiments detailed below. The data that has been generated is shown in the corresponding Figures and is also described in the corresponding Figure legends.

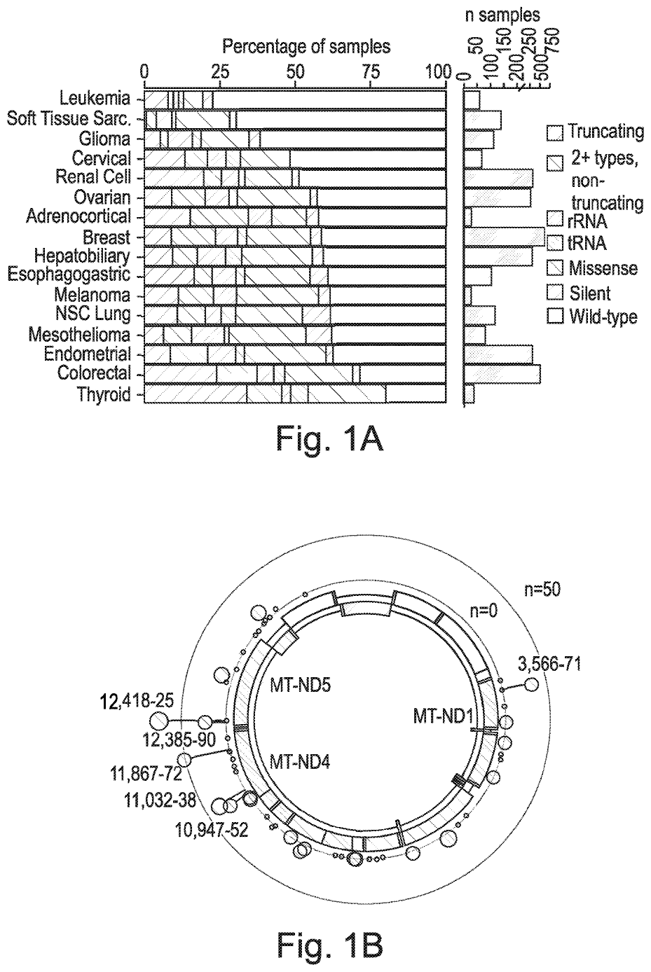

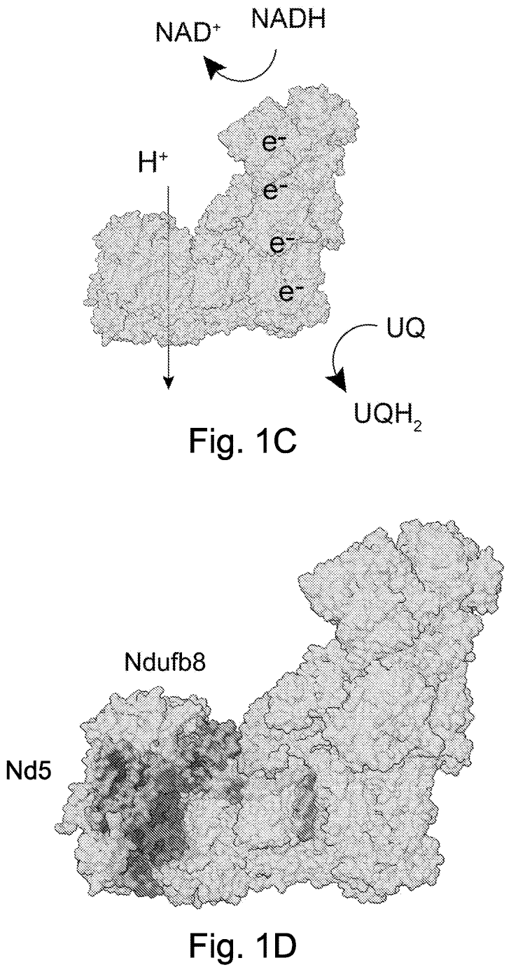

The inventors have previously identified that mtDNA mutations are abundant in cancer (see FIG. 1 herein, obtained from data in Gorelick et al., 2021). Interestingly, they observed high levels of truncating mutations with recurrence at specific positions in mtDNA that have not previously been observed. The majority of these are in complex I genes (MT-ND)—with ND5 being the most commonly impacted. Complex I is the part of the respiratory chain, oxidizing NADH to NAD+ and transferring these electrons to ubiquinone (Q) in a two electron reduction to produce ubiquinol (QH2), the energy of which is coupled to pumping proton across the mitochondrial inner membrane.

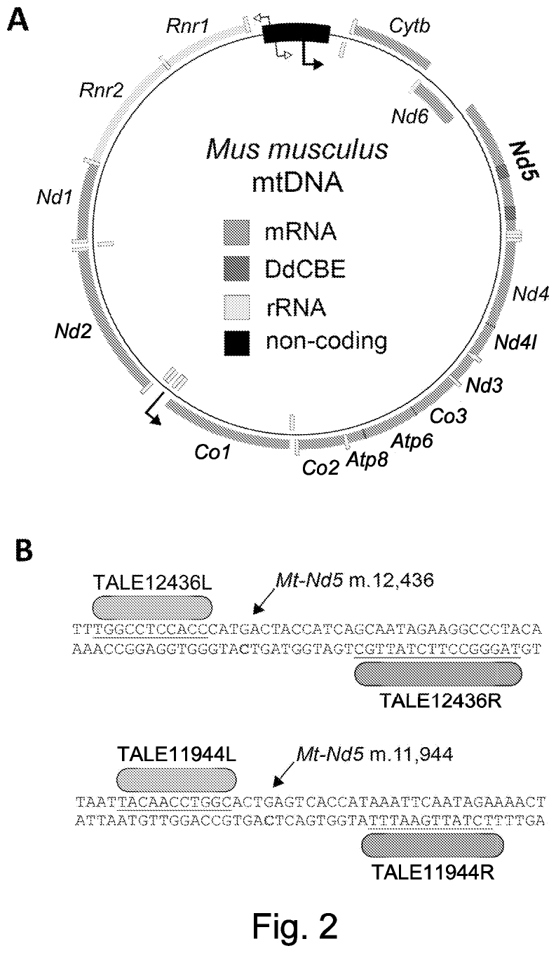

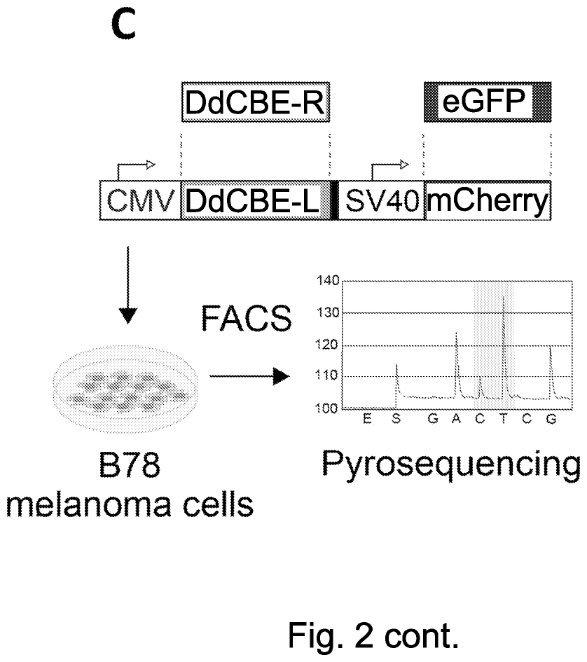

To study the role of truncating mutations in ND5, the inventors have now designed DdCBEs (mitochondrial base editing enzymes) to regions in ND5 where premature stop codons could conceivably be introduced (see FIG. 2). After screening a library of candidates in B78 melanoma cells, the inventors identified reagents which efficiently mutated m.12,436 G>A and m.11,944 G>A in Mt-Nd5, both of which convert a tryptophan codon into an in frame stop codon (TGA>TAA).

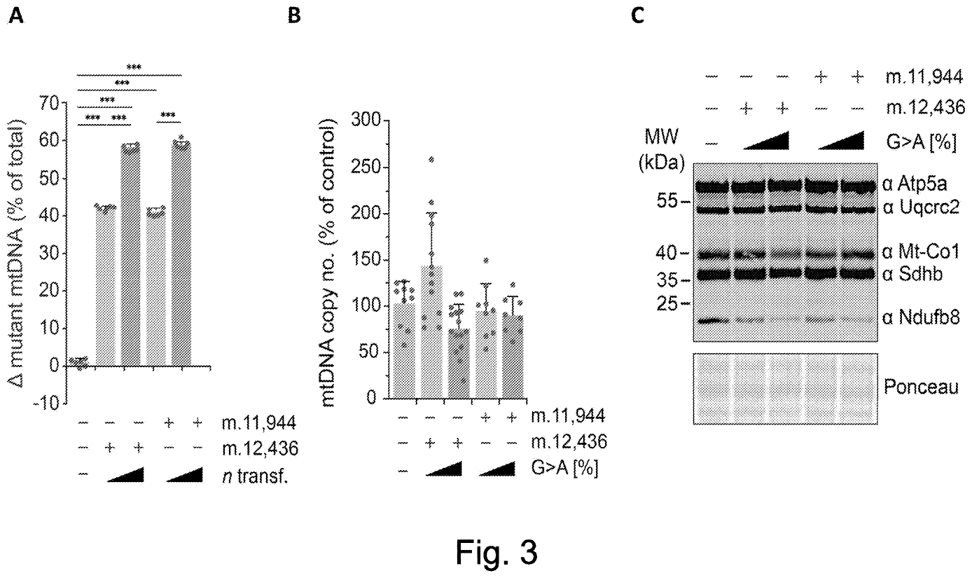

Using these reagents the inventors were able to produce cell lines with either a ~40% or a ~60% heteroplasmy (or variant allele frequency, VAF) of both of the truncating mutations (see FIG. 3). These mutations did not impact mtDNA copy number, however a mutation dose-dependent loss of Ndufb8 protein expression was observed, as would be predicted if lower levels of Mt-Nd5 were produced.

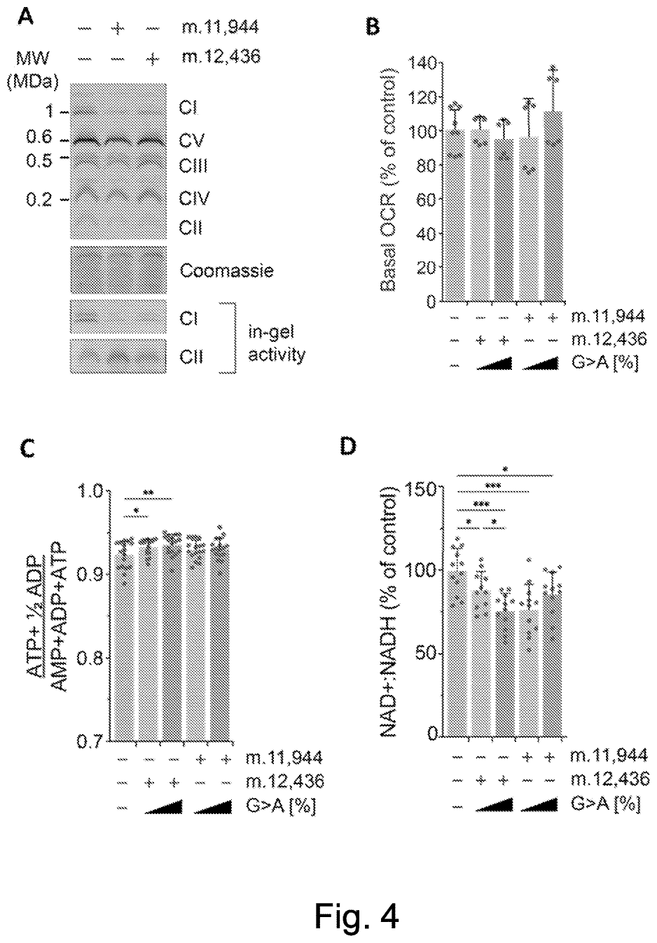

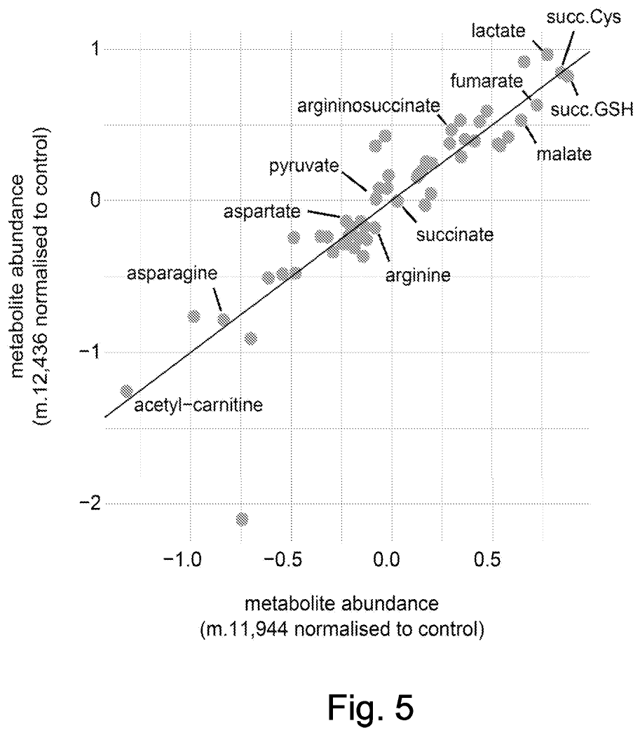

Using native gels the inventors determined that intact complex I levels in 60% VAF cells were diminished in comparison with parental cells (see FIG. 4). However, oxygen consumption (OCR) was unaffected, and adenylate charge was also not negatively impacted, suggesting that these cells are not in energetic crisis. Importantly, these cells demonstrated an altered NAD+:NADH ratio, which is to be expected as complex I is a major cellular site of NADH oxidation. A comparison of 60% VAF cells using metabolomic profiling was carried out and revealed equivalent changes in metabolic profiling between the two genetically distinct models of Mt-Nd5 truncation (see FIG. 5).

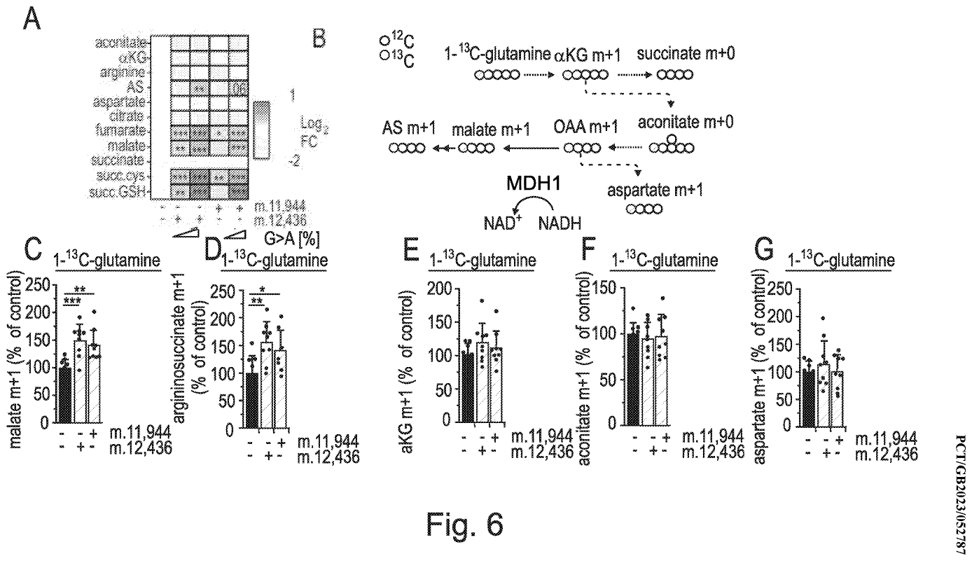

Changes in TCA cycle metabolites potentially related to the cytosolic component of the malate-aspartate shuttle (MAS) and fumarate handling led the inventors to assess the components of the MAS within mitochondria as well as assessing these components within the cytosol (see FIG. 6). To study the MAS from the cytosol, the inventors used 1-¹³C-glutamine tracing, which revealed that NAD+:NADH ratio changes are associated with increases in malate m+1 abundance, and argininosuccinate m+1 abundance, but not a-KG m+1, aconitate m+1 or aspartate m+1—implicating increased MDH1 flux rather than a) increased reductive carboxylation of glutamine or b) increased generation of arginosuccinate (then subsequently fumarate and malate) from aspartate that contributes to increased steady-state abundance of malate.

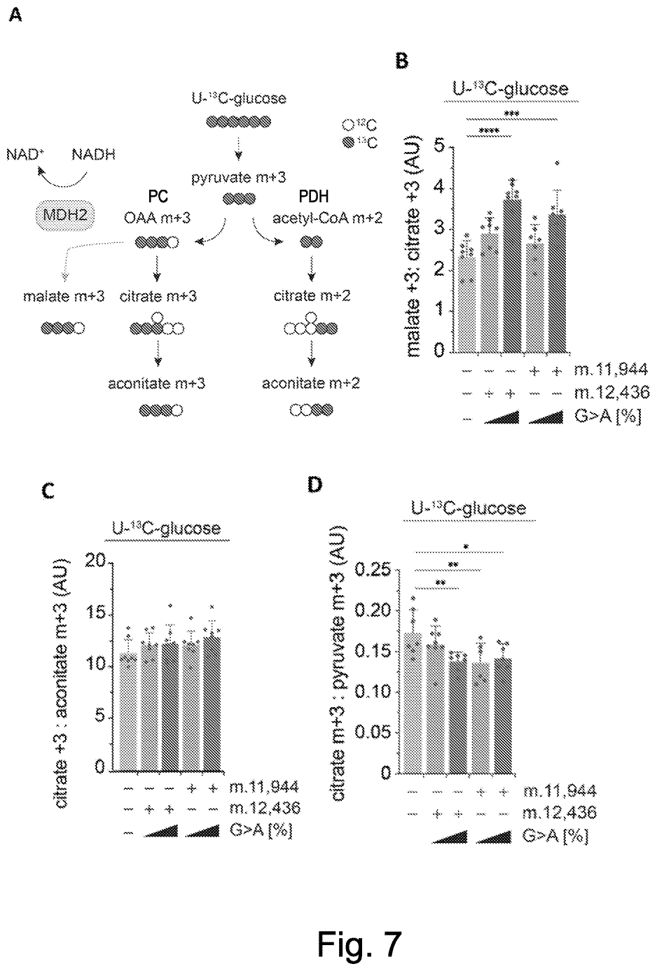

Changes in TCA cycle metabolites potentially related to the cytosolic component of the malate-aspartate shuttle (MAS) and fumarate handling led the inventors to assess the components of the MAS from within mitochondria as well as assessing these components within the cytosol. Using U-¹³C-glucose the results indicated that disturbed NAD+:NADH ratio results in partial reverse flux of MDH2 within mitochondria, determined from the ratio of pyruvate carboxylase-derived (m+3) malate, citrate, and aconitate and pyruvate (see FIG. 7).

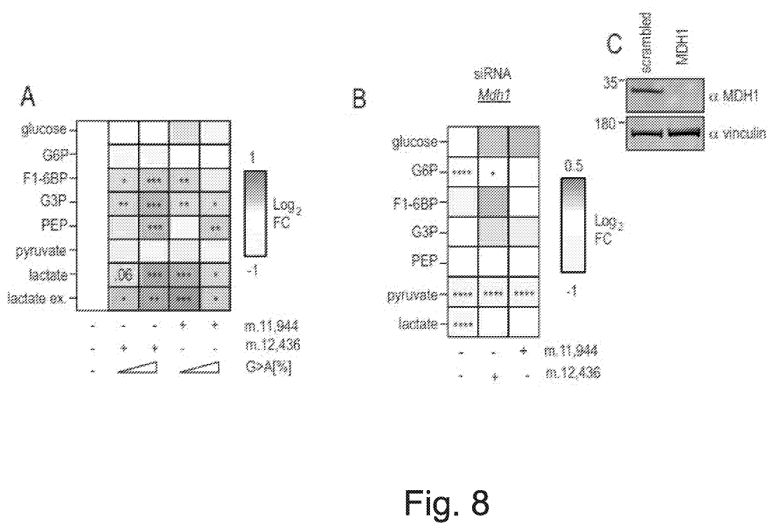

ND5 mutations in these cells are also associated with increased abundance of glycolytic intermediates (see FIG. 8, particularly FIG. 8A). MDH1 has previously been described to facilitate (by unknown means but likely physical interaction) NADH shuttling between GAPDH and MDH1. The inventors suspected that elevated cellular NADH could conceivably be counterbalanced by MDH1 regenerating NAD+through enhanced oxidation of glucose and via the interaction with GAPDH. When Mdh1 is knocked down using siRNA (see FIG. 8B), substantial changes in glycolytic intermediate abundances are observed, further implicating NAD+:NADH imbalance-driven MDH1 activity in supporting the enhanced glycolytic intermediate abundances seen in FIG. 8A.

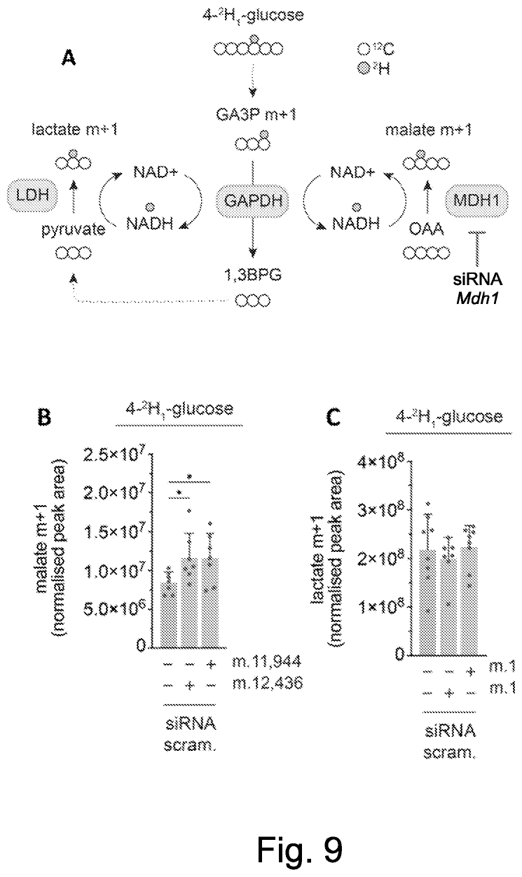

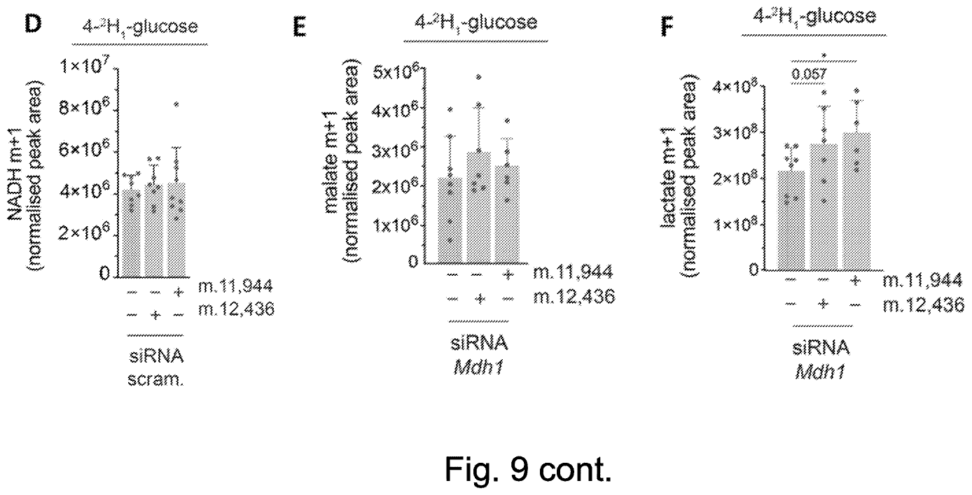

Using 4-²H₁ glucose tracing the inventors demonstrated that there is preferential shuttling of electrons from glucose to malate in Mt-Nd5 mutant cells, which is not due to increased levels of cellular NADH m+1 (see FIG. 9). Lactate m+1 is unaffected. This preferential shuttling is abolished upon knockdown of MDH1. Knockdown of MDH1 is also associated with decreased glycolytic flux (as shown in FIG. 8) and proportionally increased labelling of lactate+1 in ND5 mutant cells, suggesting that GAPDH may also shuttle electrons to LDH in specific circumstances.

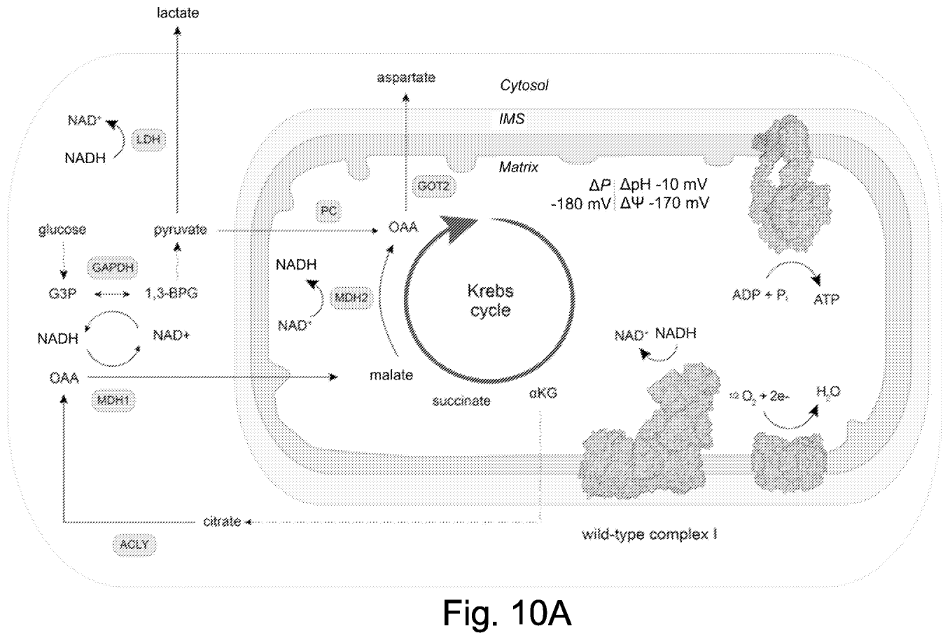

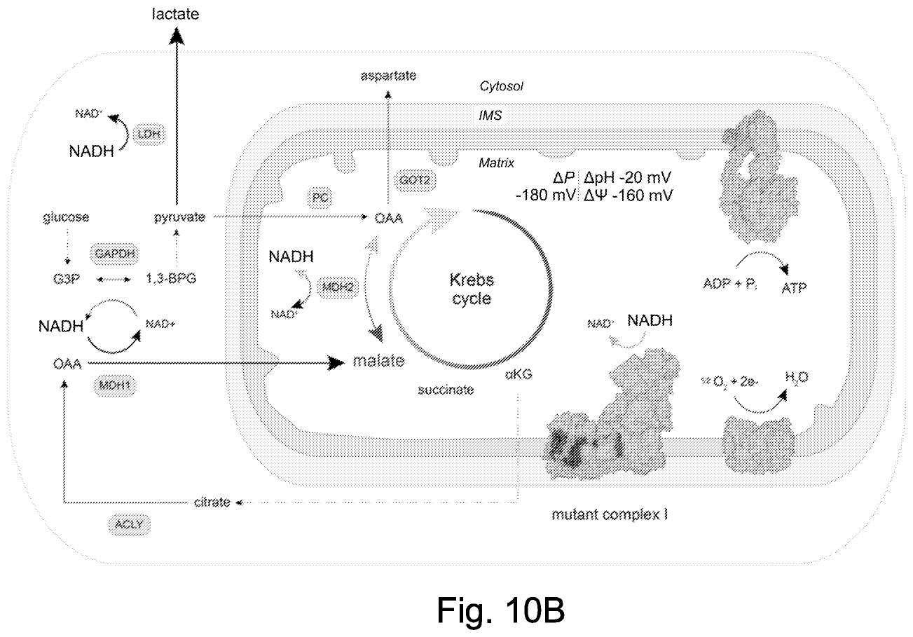

FIG. 10A shows a summary of the relevant metabolic pathways. A summary of how these change when Mt-Nd5 truncations are present is shown in FIG. 10B. Briefly, high NADH results in reverse flux of MDH2 and accumulation of cytosolically produced malate via MDH1. This results in elevated fumarate which results in increased argininosuccinate synthesis and production of fumarate adducts succinicGSH/succinylCysteine (not shown in schematic for clarity). The increased MDH1 activity drives glycolysis and results in excess glucose consumption/lactate release. Oxygen consumption and ATP synthesis remain unaffected at 60% VAF, although they are likely to be impacted at higher VAF.

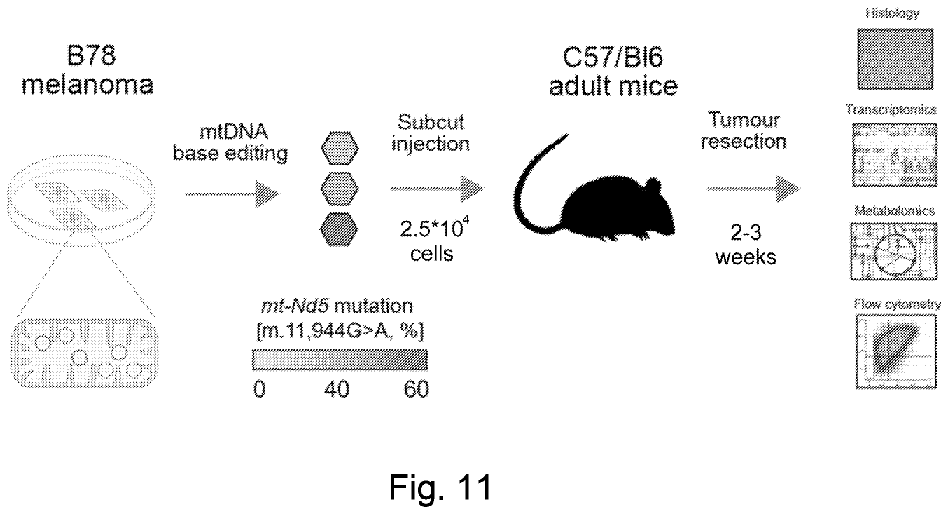

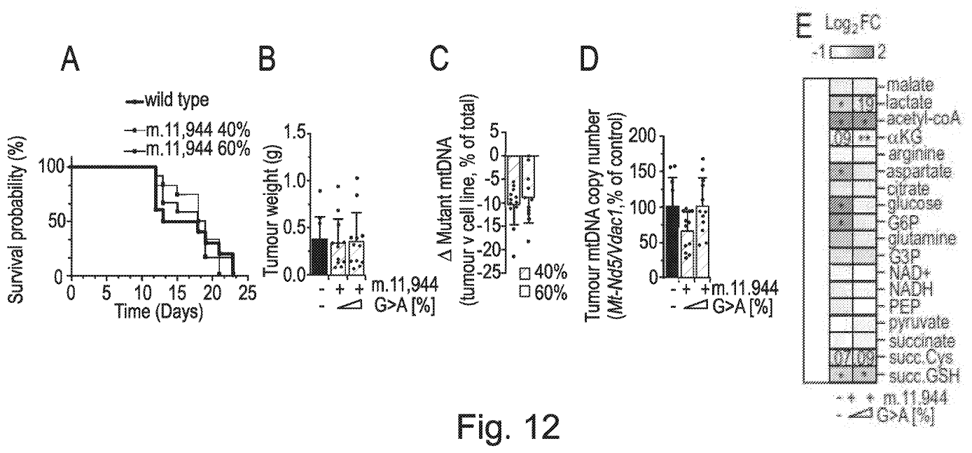

The inventors then characterized murine melanoma cells in vivo by implanting them into immunocompetent mice and allowing tumours to form (see experimental plan in FIG. 11). No gross differences in time to endpoint or tumour weight were observed, between wildtype (WT) and mutant, or between different VAFs. The difference in VAF between the implanted cells and the resulting tumours (estimated from bulk DNA extraction) is not VAF dependent and doesn't show clear selection (downward trend likely due to stromal contamination), and there is no observable difference in mtDNA copy number of the tumours either, however there are clear metabolic changes between both low and high VAF tumours and control (see FIG. 12). Of particular note are succ. Cys and succ. GSH.

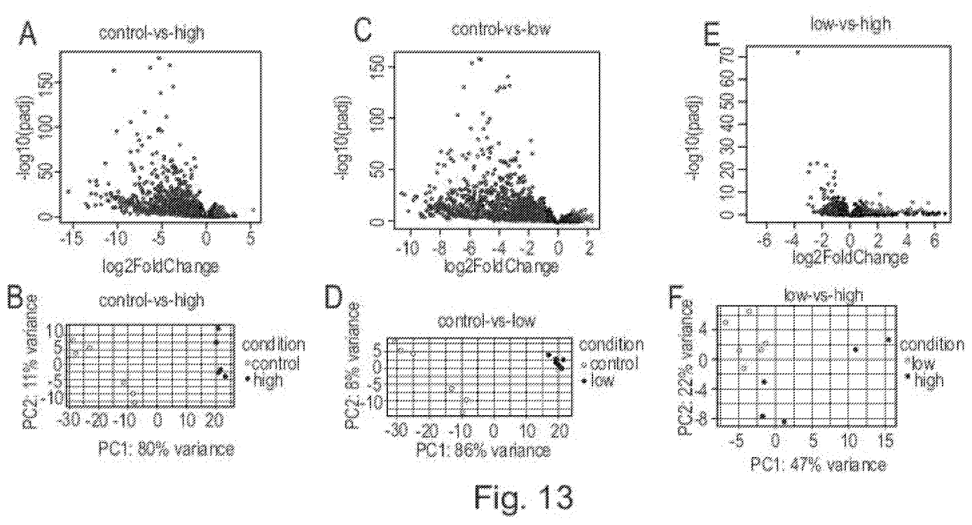

While the tumours do not appear to be different by measures used so far, when examined by bulk RNAseq there are clear and substantial differences in transcriptional profiles between control and high VAF, and control and low VAF tumours. There are more modest, but still some significant changes, between low VAF and high VAF tumours (see FIG. 13).

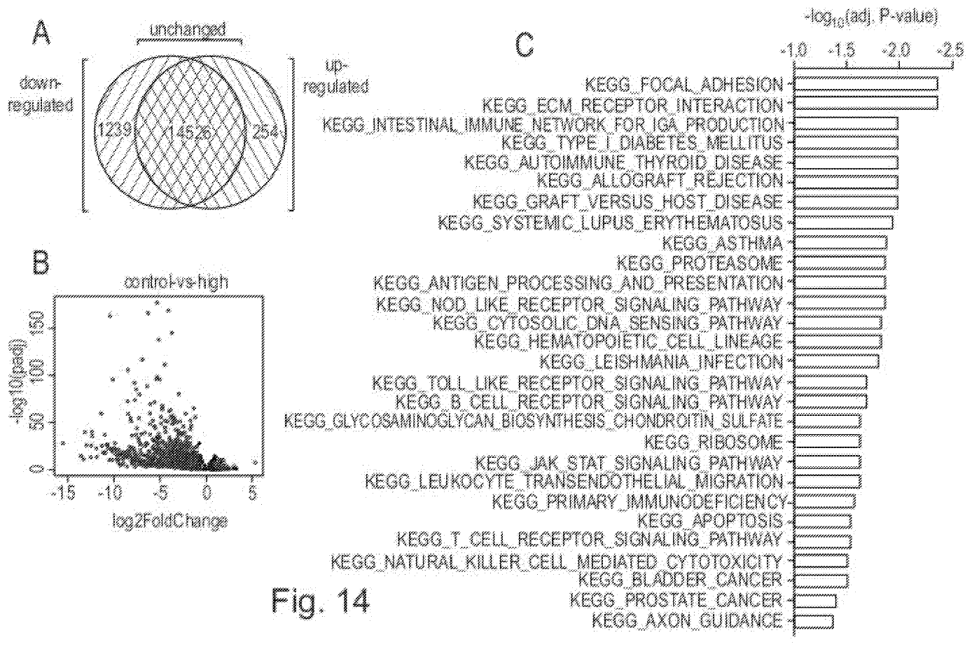

Comparing control and high VAF transcriptional data using geneset enrichment analysis revealed a host of processes that are differentially regulated, mainly relating to cell-cell interactions, receptor signalling and the immune system (see FIG. 14). Of particular note, Natural Killer Cell Mediated Cytotoxicity.

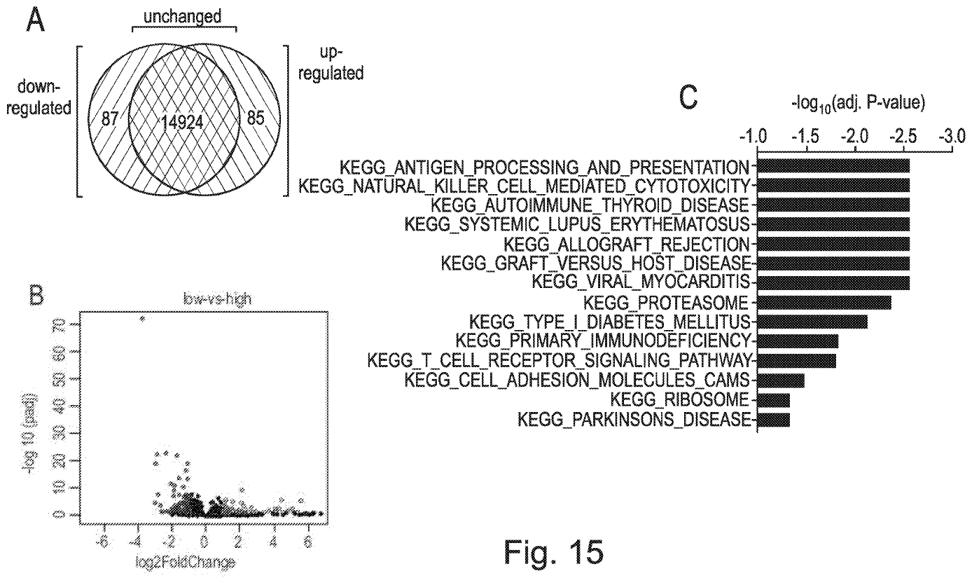

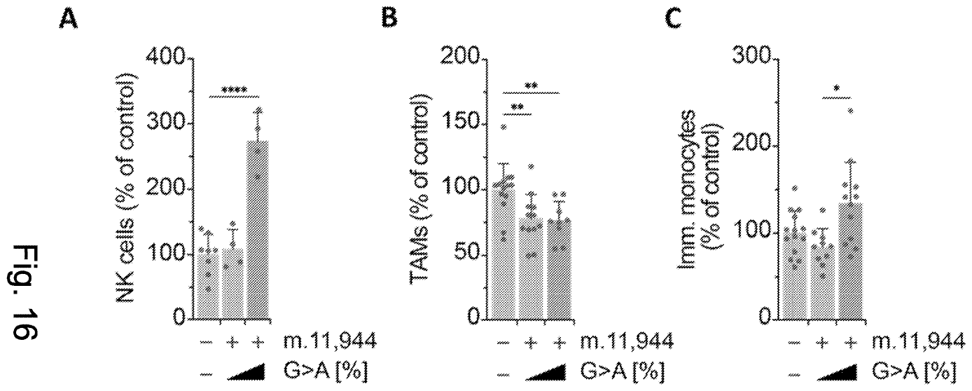

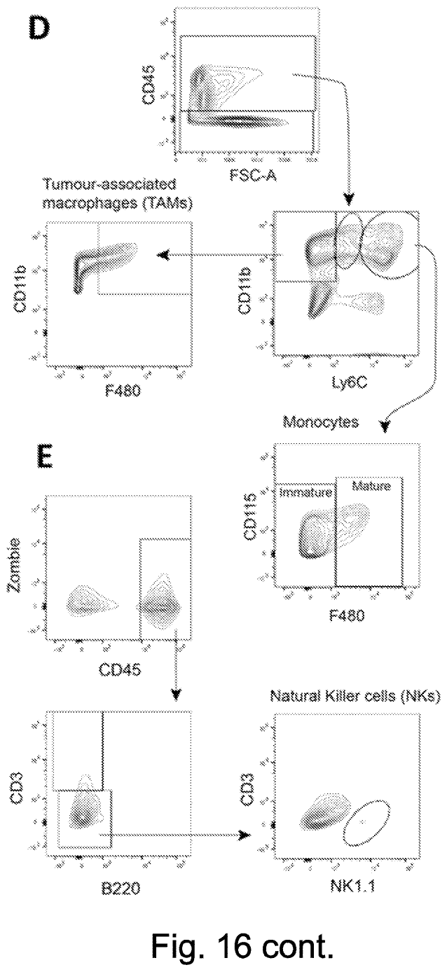

When comparing low and high VAF tumours, the list of significantly altered GSEA outputs is much shorter, and is now focused more broadly on the immune system (see FIG. 15). Again, of particular note is Natural Killer Cell Mediated Cytotoxicity.

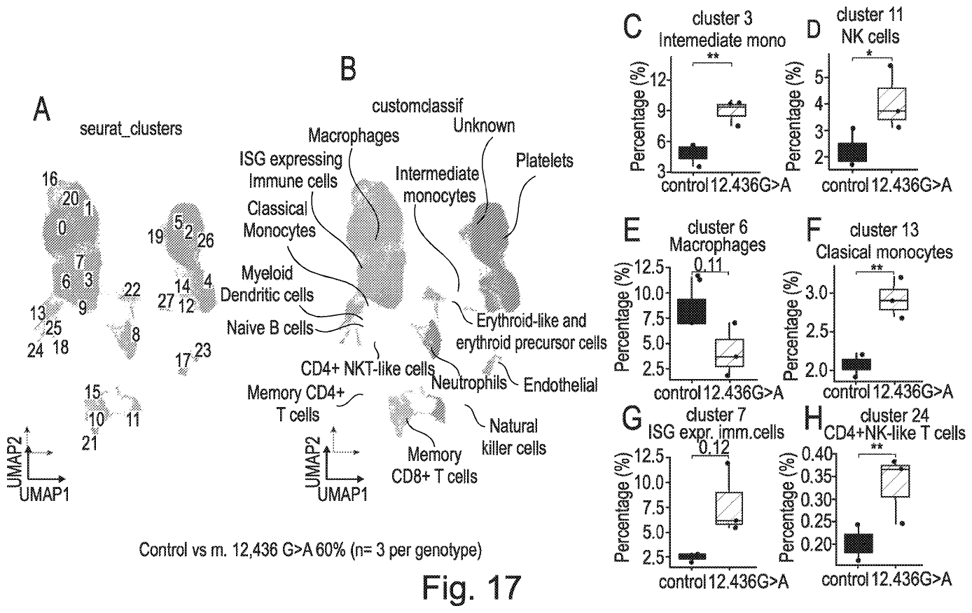

The inventors then profiled tumours using flow cytometry (see FIG. 16). Significantly altered populations are shown—NK cells, TAMs and Immature monocyte tumour residency appear to be differentially modulated by tumour mtDNA VAF. Single cell RNA sequencing further supported these data, demonstrating multiple macrophage, monocyte and NK cell resident populations that are differentially regulated by presence of high VAF mtDNA mutation (see FIG. 17).

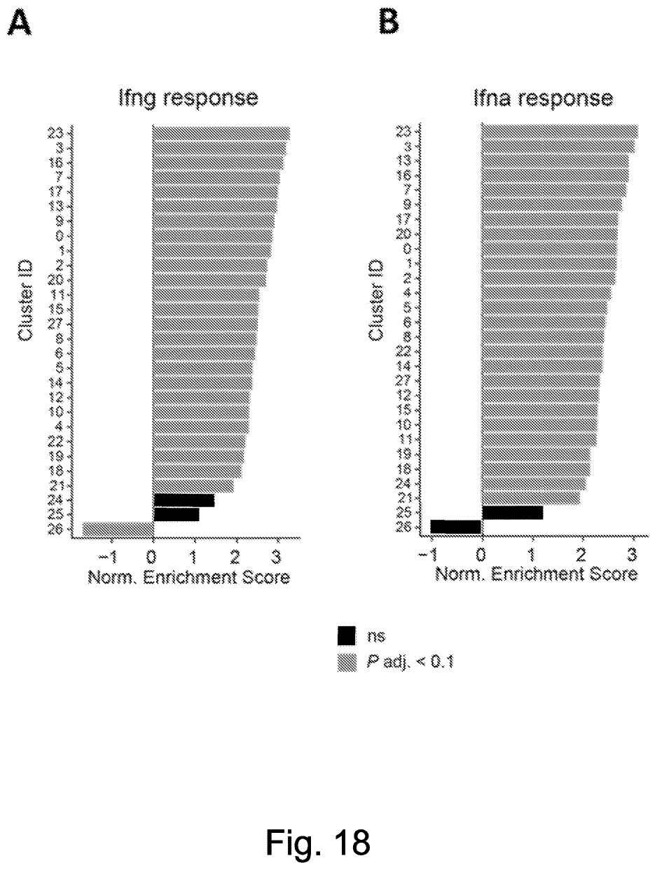

These changes in resident immune cells is coupled to a pan tumour interferon stimulated gene response (see FIG. 18), which is thought to be due to natural killer cells and CD4+NK-like T cells are the predominant source of interferon gamma. Interestingly, the only cell populations not demonstrating interferon gamma response are cluster 24 and 25, which are CD4+NK-like T cells and myeloid dendritic cells respectively. Dendritic cells are also the major source of interferon alpha, and interestingly cluster 25 is also one of only two populations not demonstrating significantly enhanced interferon alpha response.

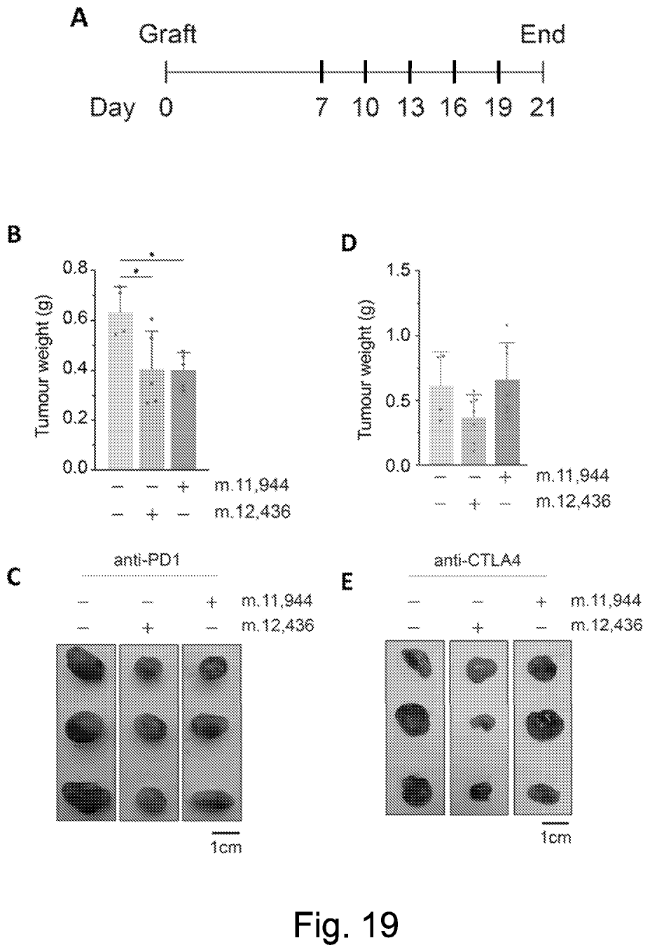

The inventors then sought to analyse the effect of this on how the mice would respond to replicate this in our mice using therapy (e.g. checkpoint blockade such as anti-PD1 treatment or anti-CTLA-4 treatment). Surprisingly, it was shown that high VAF mtDNA mutant melanoma tumours are differentially sensitive to anti-PD1 monoclonal antibody treatment in this extremely aggressive model of murine melanoma (FIG. 19). However, they are not sensitive to treatment with anti-CTLA4 monoclonal antibody treatment.

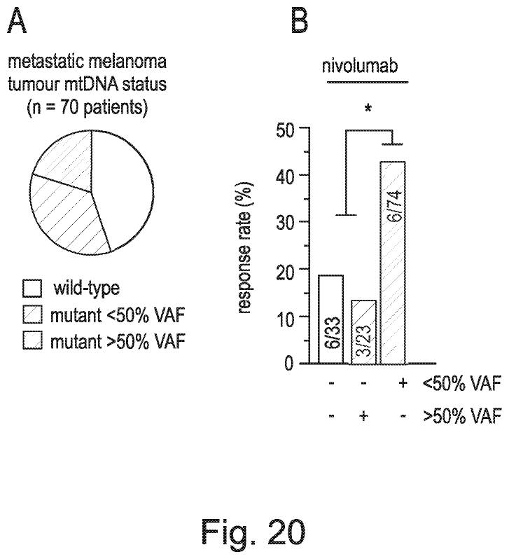

The inventors then assessed whether this would be associated with clinical outcome. Taking a small clinical cohort study (Riaz et al., 2017) they identified mtDNA mutant tumours with >50% VAF of pathogenic mtDNA mutations and calculated that these are 2.5× more likely to respond to nivolumab (anti-PD1) immunotherapy than mtDNA wild-type or low VAF tumours, with 40% of >50% VAF tumours responding compared with 17% of <50% VAF tumours responding (see FIG. 20). Of the total metastatic melanoma cohort treated with nivolumab (n=70) only 15 patients had a response to therapy. Of those 15, 12 were partial responses and 3 were complete responses. Surprisingly, 2 out of the 3 complete responders were categorised herein as >50% VAF.

The inventors have therefore identified a novel way of identifying cancer subjects that are likely to benefit from anti-PD1 therapy.

### Material & Methods

### 1. Maintenance of Cell Lines

B78 melanoma cells were cultured in standard Dulbecco's Modified Eagle Medium (DMEM) (Gibco), which contains 4.5 g/L glucose and 110 mg/L sodium pyruvate, with 20% fetal bovine serum (FBS) (Gibco), 1% Penicillin-Streptomycin (Gibco), 1× GLUTAMAX (Gibco) and 100 μg/mL uridine (Sigma). Cells were incubated at 37° C. and 5% CO₂ and split when ~80% confluent.

### 2. Animal Models

All animal experiments were carried out in accordance with the UK Animals (Scientific Procedures) Act 1986 under PPL P72BA642F. The C57BL/6 mice were used for all studies and housed in up to 5 per cage in a temperature-controlled (21° C.) room with a 12-h light-dark cycle. Male mice of an average age of 6 weeks were used.

0.25×10⁶ cells were resuspended 1:1 in RPMI (Gibco) and Matrigel® Matrix (Corning) in 50 μL. Cells were injected over the flank and mice were culled at the tumour endpoint of 15 mm. Mice receiving checkpoint blockade therapy were dosed with 200 mg of Ultra-LEAF™ Purified anti-mouse CD279 (PD-1) (Biolegend) via intraperitoneal injections. Mice were dosed at 7 days post-injection of tumour cells and dosed twice a week till day 21 post-injection when they were culled, and tumours harvested.

### 3. Constructs and Plasmids

Transcription activation-like effector (TALE) domains were designed. TALE domains were cloned into either a pcmCherry or pTracer backbone.

4. Primers and siRNA

### PyroMark Assay Primers for Measuring Mt.11944 Heteroplasmy

Forward:
(SEQ ID NO: 1)
CTTCATTATTAGCCTCTTAC
Reverse:
(SEQ ID NO: 2)
GTCTGAGTGTATATATCATG
Sequencing:
(SEQ ID NO: 3)
CTATTGAATTTATGGTGACT

### PyroMark Assay Primers for Measuring Mt.11944 Heteroplasmy

Forward:
(SEQ ID NO: 4)
ATATTCTCCAACAACAACG
Reverse:
(SEQ ID NO: 5)
GTTATTATTAGTCGTGAGG
Sequencing:
(SEQ ID NO: 6)
CTATTGCTGATGGTAGT

ddPCR EvaGreen Primers

ND5 Forward:
(SEQ ID NO: 7)
TGCCTAGTAATCGGAAGCCTCGC
ND5 Reverse:
(SEQ ID NO: 8)
TCAGGCGTTGGTGTTGCAGG
VDAC1 Forward:
(SEQ ID NO: 9)
CTCCCACATACGCCGATCTT
VDAC1 Reverse:
(SEQ ID NO: 10)
GCCGTAGCCCTTGGTGAAG

siRNA for Metabolomics Experiments
ON-TARGETplus Mouse MDH1 siRNA—SMARTPool (L-051206-01-0005)
ON-TARGETplus Non-targeting Control Pool (D-001810-10-05)

### 5. Antibodies

### Primary and Secondary Antibodies for Immunoblotting and BN-PAGE

Total OXPHOS Rodent WB Antibody Cocktail (ab110413) used 1:800
MDH1 Polyclonal Antibody (15904-1-AP) used 1:1000
IRDye® 800CW Goat anti-Rabbit IgG (Licor) used 1:10,000
IRDye® 680RD Donkey anti-Mouse IgG (Licor) used 1:10,000

### Antibodies for Flow Cytometry

All antibodies were purchased from Biolegend and are anti-mouse.

TABLE 1
Neutrophil, Eosinophil, Monocyte and Macrophage Panel

| Target | Fluorophore |
|---|---|
| CD45 | A700 |
| CD11b | FITC |
| F4180 | BV510 |
| CD11c | BV785 |
| MHCII | BV421 |
| Ly6c | Perpcy5.5 |
| CD115 | BV711 |
| CX3CR1 | BV605 |
| SiglecF | A647 |
| Gp38 | PE |
| CD31 | Pecy7 |
| Epcam | BV650 |

TABLE 2
T-cell Panel

| Target | Fluorophore |
|---|---|
| CD3 | BV605 |
| CD45 | BV421 |
| Nk1.1 | Perpcy5.5 |
| PD-1 | BV510 |
| CD69 | BV650 |
| CD44 | BV711 |
| CD8 | FITC |
| CD4 | Pecy7 |
| B220 | BV785 |
| CD622 | A700 |
| CD27 | APC |
| CD3 | BV605 |

### 6. Cell Transfection and FACS

B78 cells were plated into 10 cm dishes to achieve ~-50% confluency on the day of transfection. 20 μg of DNA was mixed with 40 μL of P3000™ reagent and combined with 30 μL of Lipofectamine™ 3000 in a final volume of 1000 μL of OptiMEM. Transfection reagents were bought from Invitrogen. A negative control was set up alongside and the mixtures incubated at room temperature for 15-20 minutes before adding to the dishes. Cells were incubated at 37° C. and 5% CO₂ for 24 hrs.

Cells were prepared for fluorescence-activated cell sorting (FACS) in 1 ml of DMEM and 1 μg/mL 4′,6-Diamidino-2-Phenylindole, Dihydrochloride (DAPI). Live cells were sorted for co-expression of mCherry and GFP and left to recover for 10 days before heteroplasmy measurements.

### 7. DNA Extraction

Cell culture medium was aspirated, and cells were washed once with PBS. Cells were detached using 1× trypsin (Gibco), re-suspended in cell culture medium and centrifuged at 300 g for 5 minutes. The pellet was re-suspended in 200 μL PBS for DNA extraction using the DNeasy Blood and Tissue Kit (Qiagen), according to the manufacturer's instructions. DNA concentration was then measured using a NanoDrop.

Tumour tissue (up to 20 mg) was treated as per the manufacturer's instructions using the DNeasy Blood and Tissue Kit (Qiagen).

### 8. Pyromark PCR

40 ng of genomic DNA extracted from cells, as per section 7, was mixed with 12.5 μL 5× PyroMark PCR Master Mix, 0.05 μL of 100 μM forward and reverse primers, 2.5 μL CoralLoad Concentrate and water to a final volume of 25 μL. All reagents were bought from Qiagen. PCR was performed according to the manufacturer's instructions with 60% C. to anneal.

The pyromark assay was designed using the PyroMark Assay Design 2.0 software. The assay was performed on the PyroMark Q48 Autoprep as per the manufacturer's instructions using 10 μL of each PCR product.

9. Digital Droplet PCR (ddPCR)

1 ng/μL sample DNA was mixed with 10 μL ddPCR Supermix for EvaGreen (2×) (BioRad), 110 nM of forward and reverse primers and water for a final volume of 20 μL per well. Samples were prepared in triplicate in a 96-well plate. The plate was sealed at 180° C. for 10 seconds using the PX1™ PCR Plate Sealer (Bio-Rad) and centrifuged briefly to remove any air bubbles. An Automated Droplet Generator (Bio-Rad) was used for droplet formation in a new 96-well plate. The plate was re-sealed and placed into a C1000 Touch Thermal Cycler (Bio-Rad) for PCR. PCR was performed according to the Bio-Rad ddPCR protocol for EvaGreen. Once completed, DNA was quantified using a QX200™ Droplet Reader (Bio-Rad).

### 10. Immunoblotting

Cultured cells were detached and spun down at 1000 g. The pellet was washed once with PBS and kept on ice. An appropriate amount of lysis buffer [10 mL radioimmunoprecipitation assay (RIPA) buffer (Invitrogen), 100 μL 1% Triton X-100 (Invitrogen) and 100 μL Halt™ Protease and Phosphatase Inhibitor Single-Use Cocktail (100×) (Invitrogen)] was added each pellet and left on ice for 10 minutes. The lysate solutions were spun down at 14,000 g for 5 minutes at 4° C. Protein quantification was done using the Pierce BCA Protein Assay Kit (Invitrogen) as per the manufacturer's instructions in a 96-well plate.

Protein samples were made to a final concentration of 100 μg in 50 μL. The appropriate amount of supernatant, based on the BCA assay, was mixed with 1:4 total volume of NuPAGE™ LDS Sample Buffer (4×) (Invitrogen) and 1:10 total volume of NuPAGE™ Sample Reducing Agent (10×) (Invitrogen). Samples were incubated at 37° C. for 20 minutes for and then loaded into Bolt™ 4-12% Bis-Tris Plus Gel (Invitrogen) with 1×MOPS buffer. PageRuler™ Prestained Protein Ladder (Invitrogen) was used. Gels were run at 180V until the dye front reached the end of the gel.

Once run, proteins were transferred onto a nitrocellulose membrane via wet transfer. The gel was placed in a ‘transfer sandwich’ in the order of: sponge, filter paper, gel, nitrocellulose membrane, filter paper and sponge. The transfer was run at 100V for 1 hour using 25 mM Tris, 192 mM glycine (pH 8.3) and 20% methanol in water as the buffer. The membrane was then washed in 1×TBST and then blocked with 5% non-fat milk in 1×TBST for 1 hour at room temperature on a roller. The solution was then replaced with the primary antibodies made in 5% non-fat milk in 1×TBST. The membrane was left overnight at 4° C. on a roller. The following day, the membrane was washed three times with 1×TBST for 5 minutes on a roller at room temperature before adding the secondary antibodies in 1×TBST. The membrane was covered and incubated on a roller for 1 hour at room temperature. The membrane was then washed three times with 1×TBST for 5 minutes before imaging on the Licor Odyssey Fc Imaging System.

### 11. Blue Native-PAGE

11.1 Mitochondrial Isolation Cells were bulked to yield ~100×10⁶ cells for mitochondrial isolation. Cells were trypsinised and pelleted into a 15 ml falcon tube. Cell pellets were washed twice in ice-cold PBS and spun at 600 g between each step before re-suspension in one-half volume of Hypotonic Buffer IB 0.1 (3.5 mM Tris-HCl pH 7.8, 2.5 mM NaCl, 0.5 mM MgCl₂). The cell suspension was homogenised with 80-100 strokes using a dounce homogeniser. 1/10^{th} the original volume of packed cells of Hypertonic Buffer IB 10 (0.35M Tris-HCl pH 7.8, 0.25M NaCl, 50 mM MgCl₂) was immediately added to the suspension and the homogenate was transferred into a clean 15 ml falcon tube. Isotonic buffer IB 1 (35 mM Tris-HCl pH 7.8, 25 mM NaCl, 5 mM MgCl₂) was used to clean the homogeniser to collect excess cells and added to the homogenate. Samples were spun down at 12,000 g for 3 minutes at 4° C. to eliminate nuclear contamination. The supernatant was transferred to a clean tube and this step was repeated to ensure minimal contamination. The supernatant was then spun down 17,000 g for 2 minutes at 4° C. to pellet mitochondria. The mitochondrial pellet was then washed using Homogenisation Media (0.32M sucrose, 10 mM Tris-HCl pH 7.4, 1 mM EDTA) and spun again. The mitochondrial fractions were then used immediately for BN-PAGE.

### 11.2 BN-PAGE Gel and Imaging

All reagents were purchased through Invitrogen.

Mitochondrial pellets were solubilised in cold 1× NativePage™ Sample Buffer with 1% Digitonin. Samples were incubated on ice for 15 minutes and then spun down at 20,000 g for 30 minutes at 4° C. Protein concentration was determined using the Pierce™ BCA Assay Kit as per the manufacturer's instructions. Samples were made up to 100 ug in 50 ul with 1×NativePage™ Sample Buffer with 1% Digitonin. Immediately prior to loading the sample, NativePAGE 5% G-250 Sample Additive was added to each sample to a final concentration of 0.5%.

NativePage™ Novex 3-12% Bis-Tris Gels were used for this experiment. The cassette was removed and the wells were washed with Dark Blue Cathode Buffer (1× NativePage™ running buffer, 1× NativePage™ Cathode Additive in water). The gels were placed securely into an XCell SureLock Mini-Cell. The outer chamber was filled with ~600 mL Anode Buffer (1× NativePage™ running buffer in 950 mL water) and ~200 mL of the Dark Blue Cathode Buffer in the inner chamber. Samples were then loaded into the wells alongside the NativeMark™ Unstained Protein Standard. The gel was run at 150V and the Dark Blue Cathode Buffer was switched for the Light Blue Cathode Buffer (1× NativePage™ running buffer, 0.1× NativePage™ Cathode Additive in water) when the dye front had travelled ~⅓^{rd} down. The gel was then left to run until the dye front had reached the bottom of the cell.

Proteins were transferred onto a PVDF membrane using the wet transfer method highlighted in section 11. The transfer buffer used in this experiment was 1× NuPage Transfer Buffer and the transfer was run at 60V for 1 hour.

The membrane was then blocked and blotted as per the method in section 10.

### 12. Seahorse Assay

The day prior to the assay, cells were plated at 20,000 cells/well in a Seahorse XF96 Cell Culture Microplate (Agilent). The outer rows and columns were left empty and cells were plated down each column. 200 μL of MQ water was also added to each well of the Seahorse XF96 sensor cartridge microplate and left alongside 50 mL of Seahorse XF Calibrant solution at 37° C. overnight.

The following day, the water from the Seahorse XF96 sensor cartridge microplate was discarded and replaced with 200 μL of the pre-heated calibrant. The cartridge was incubated at 37° C. for 45-60 minutes. Oligomycin, FCCP, rotenone and antimycin A were added to separate ports in the seahorse cartridge to a final concentration of 1 mM. The cartridge was then placed into the Seahorse XF96 Analyser for calibration.

In parallel, the cell culture plate was washed oncewith PBS. 150 μL of Seahorse media (25 mM glucose, 1 mM sodium pyruvate, 2 mM L-glutamine and 1% FBS in Seahorse XF Media) was added to each well and incubated at 37° C. for 30 minutes. The plate was then placed into the Seahorse XF96 Analyser following successful calibration. Oxygen consumption rate and extracellular acidification rate were measured using a Mito Stress template from the manufacturer's website.

### 13. Metabolomics

### 13.1 Experimental Media

Steady state metabolomics experiments used cell culture media, as per section 1, containing 2 mM L-glutamine (Gibco) in place of 1× GLUTAMAX. Plasmax was bought from Ximbio and supplemented with 2.5% dialysed FBS for these experiments.

U-¹³C glucose and 4-²H glucose were prepared using DMEM, no glucose (Gibco) and supplemented with 20% FBS, 1 mM sodium pyruvate (Gibco), 100 μg/mL uridine and either 25 mM U-¹³C glucose or 4-²H glucose.

U-¹³C glutamine and 1-¹³C glutamine medium was prepared using standard DMEM supplemented with 20% FBS, 100 μg/mL uridine and either 4 mM U-¹³C glutamine or 1-¹³C glutamine.

### 13.2 Intracellular and Media Metabolite Extraction

Cells were plated in triplicate in 12-well plates to achieve ~70-80% confluency for the day of extraction. The following day, the media was aspirated and replaced with experimental media and the cells were incubated for 24 hours for extraction the following day.

All work was conducted on ice to reduce significant metabolite changes. 20 μL of media from each well was added to 980 μL ice-cold extraction media (50% LC/MS grade methanol, 30% LC/MS grade acetonitrile, 20% LC/MS grade water) for media analysis. The media from each plate was then quickly removed and washed twice with ice cold PBS. The plate was tapped on tissue paper and remaining PBS aspirated before adding 200 μL ice-cold extraction media to each well. The plate was stored at 4° C. and the extraction media was transferred into ice-cold microcentrifuge tubes. Samples were spun down at 14,000 g for 10 minutes at 4° C. before transferring into screw vials. Samples were stored at −80° C. until run on the mass spectrometer by the in-house facility.

Traces from all experiments were analysed using Tracefinder 4.0.

### 13.3 Normalisation of Samples

Plates used for the extractions were air-dried at room temperature and stored at 4° C. for a maximum of two weeks until the protein assay was performed.

The Lowry assay was used to measure protein concentration in each well. Briefly, 200 μL of Solution A (05% sodium deoxycholate and 1M sodium hydroxide in water) was added to each well, as well as an additional plate with a BSA standard curve, and left to shake vigorously at room temperature for 40 minutes. 2 mL of Solution B (0.629 mM copper disodium ethylenediaminetetraacetate, 189 mM sodium carbonate and 200 mM sodium hydroxide) and shake the plates for 10 minutes. 200 μL of Folin & Ciocalteu's phenol reagent (Sigma) was then added to each well and incubated at room temperature on a shaker for 40 minutes. 200 μL from each well was then transferred to a 96-well plate and the absorbance at 750 nm was read using a SpectraMax ABS Plus (Molecular Devices). The protein concentrations were calculated from the standard curve and used for normalisation of traces.

13.4 siRNA Knockdown

12,00 cells were plated per well of a 12-well plate for each siRNA experiment. Each condition was plated in triplicate. The following day, for each well, 5 μL of 5 μM siRNA was added to 95 μL Opti-MEM. In a separate tube, 5 μL DharmaFECT 1 Transfection Reagent (Horizon Discovery) was added to 95 μL Opti-MEM. The tubes were left at room temperature for 5 minutes to equilibrate before mixing. Samples were left at room temperature for 15-20 minutes. 800 μL of standard media was then added to the suspension and added to the cells. Experimental media was added 48 hours later and extractions were done as per section 14.2.

### 14. Cell and Tissue Bulk Transcriptomics

1×10⁶ cells were pelleted into 1.5 ml microcentrifuge tubes and stored at −80° C. Tumour tissue (~20 mg) was stored in RNAlater™ Stabilisation Solution (Invitrogen) and kept at −80° C. Samples were then sent on dry ice to Azenta for sample processing, sequencing and analysis.

### 15. Flow Cytometry

Harvested tumours (~30 mg) were chopped and re-suspended in digestion buffer (500 U/mL collagenase I, 100 U/mL collagenase IV and 0.2 mg/mL DNase I in RPMI). Samples were incubated at 37⁰ in a shaking rotor for 40 minutes. Samples were then passed through a 40 μm filter and spun down at 800 g for 3 minutes to pellet cells. Cells were re-suspended in 200 μL FACS buffer and split across two wells of a round-bottomed 96-well plate. The plate was spun down at the same speed and the supernatant was thrown off. Cell pellets were re-suspended in 100 μL 1:1000 Zombie Aqua (BioLegend) in PBS. The plate was kept at 4° C. for 20 minutes.

The plate was re-spun and cell pellets were re-suspended in 100 μL of each flow panel made in FACS buffer, as outlined section 6. The plate was kept at 4° C. for at least 60 minutes. The plate was re-spun and the cell pellets were then re-suspended in 100 μL 4% Pierce™ 16% Formaldehyde (Invitrogen) and incubated at room temperature for 10 minutes. The plate was spun again, and samples were re-suspended in 100 μL of FACS buffer. The plate was wrapped in Parafilm and aluminium foil and kept at 4° C. for a maximum of 2 weeks.

Fixed samples were re-suspended in FACS buffer and moved to FACS tubes when needed to be run. 10×10⁶ events were recorded per sample on the Fortessa and analysis was done using FlowJo.

### 16. Tumour Single-Cell RNA Sequencing

Tumour tissue was digested as per section 15. Cells were then re-suspended in 1 ml of FACS buffer (2% FBS and 0.5 mM EDTA in PBS) with 1 μg/mL DAPI. Live cells were sorted and submitted to the in-house facility for barcoding using 10× Genomics Chromium platform and 3′ library prep kit. Sequencing was carried out at Glasgow Polyomics facility. Single-cell sequencing reads were aligned against the mouse GRCm39 reference genome using CellRanger (version 7.0.1), and the expression raw count matrixes were analyzed using the Seurate package (version 4.0.6). The malignant tumour cells and non-malignant cells were further separated based on the genome-wide copy number landscape changes estimated by the copykat package (version 1.1.0). The cell type of each cluster identified in the Seurate workflow was annotated using SingleR (version 1.10.0) through the gene expression correlation comparisons with known cell types in the mouse reference dataset. Differential gene expression analyses were carried out on log-normalized gene expression using the MAST algorithm within the FindMarkers function in Seurate.

### 17. Riaz et al Clinical Trial Data Analysis

Aligned reads for ChrM for patients' tumour and normal samples were acquired and processed according to previously described pipelines which are now publicly available (see e.g. Gorelick et al., Nature Metabolism 2021). Mutation calls and calculated heteroplasmy were used to assign patients to mtDNA wildtype, <50% VAF or >50% VAF groups.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### Sequences

### PyroMark Assay Primers for Measuring Mt.11944 Heteroplasmy

Forward:
(SEQ ID NO: 1)
CTTCATTATTAGCCTCTTAC
Reverse:
(SEQ ID NO: 2)
GTCTGAGTGTATATATCATG
Sequencing:
(SEQ ID NO: 3)
CTATTGAATTTATGGTGACT

### PyroMark Assay Primers for Measurinq Mt.11944 Heteroplasmy

Forward:
(SEQ ID NO: 4)
ATATTCTCCAACAACAACG
Reverse:
(SEQ ID NO: 5)
GTTATTATTAGTCGTGAGG
Sequencing:
(SEQ ID NO: 6)
CTATTGCTGATGGTAGT

ddPCR EvaGreen primers

ND5 Forward:
(SEQ ID NO: 7)
TGCCTAGTAATCGGAAGCCTCGC
ND5 Reverse:
(SEQ ID NO: 8)
TCAGGCGTTGGTGTTGCAGG
VDAC1 Forward:
(SEQ ID NO: 9)
CTCCCACATACGCCGATCTT
VDAC1 Reverse:
(SEQ ID NO: 10)
GCCGTAGCCCTTGGTGAAG

### REFERENCES

Gorelick et al., 2021, Nature Metabolism April; 3(4):558-570.
Riaz et al., 2017 doi: 10.1016/j.cell.2017.09.028. Epub 2017 Oct. 12.

### Example 2

### Results

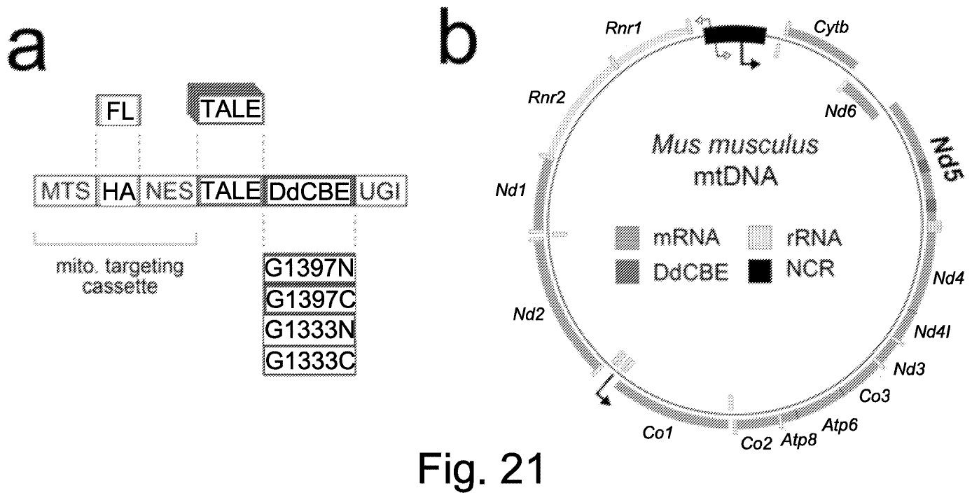

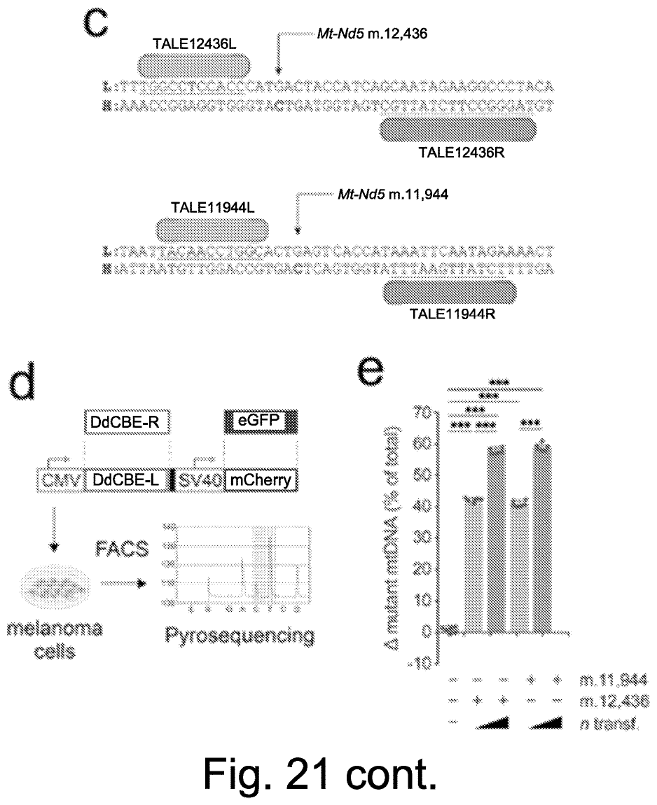

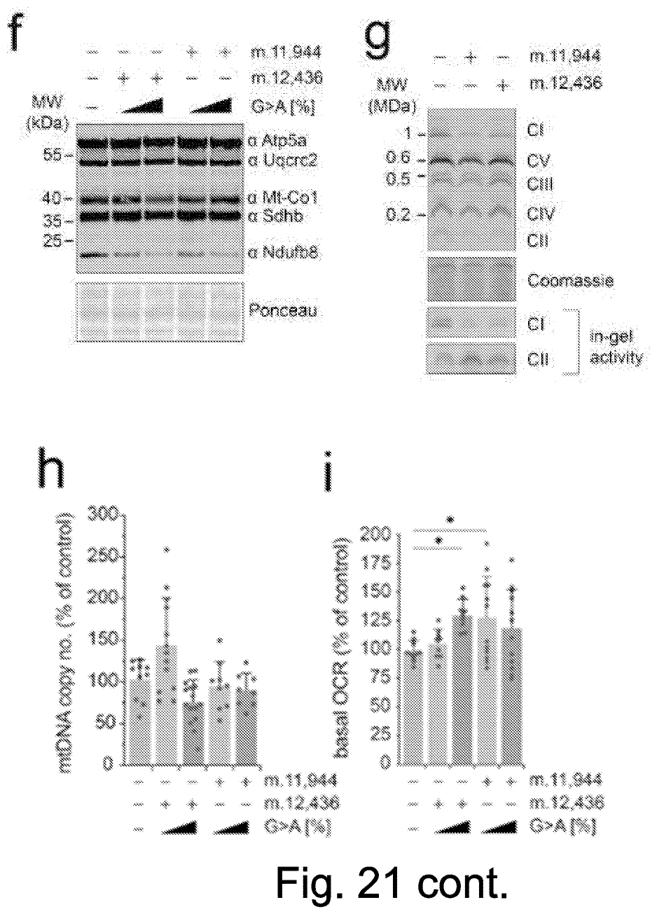

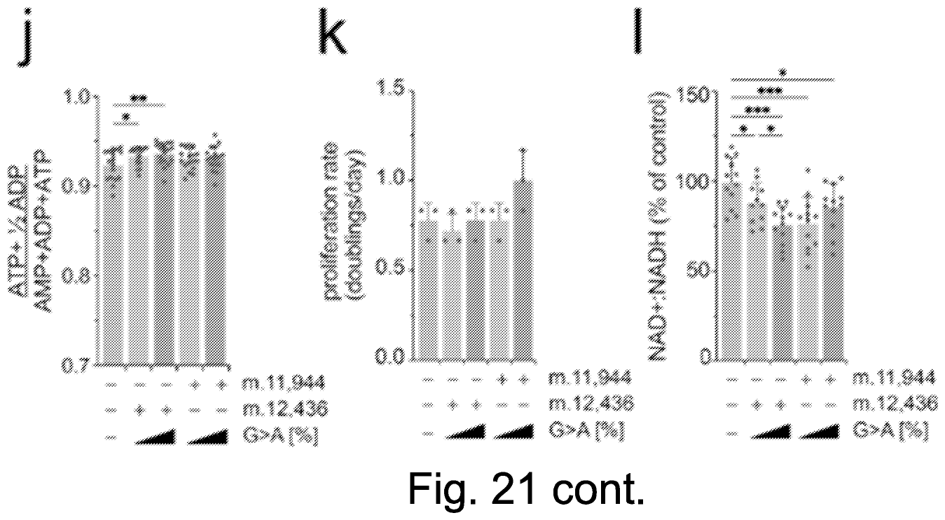

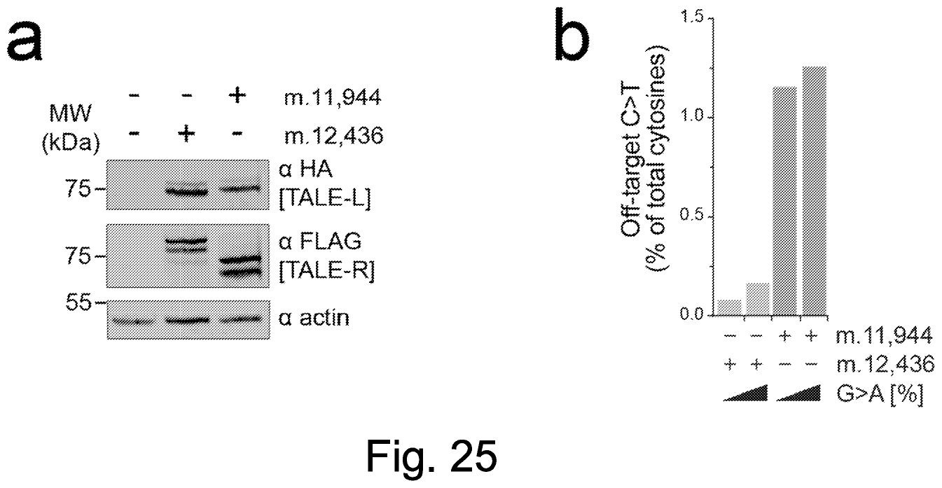

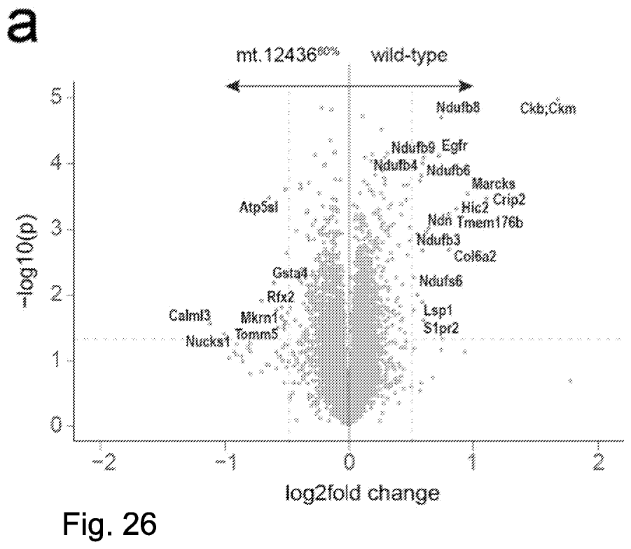

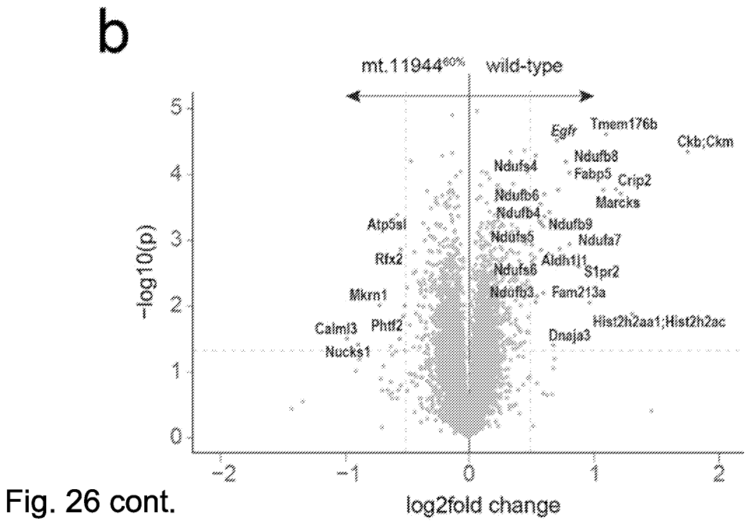

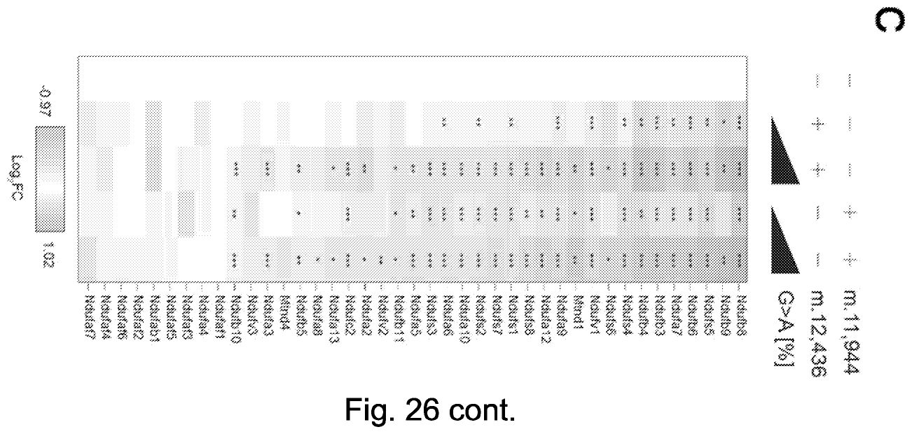

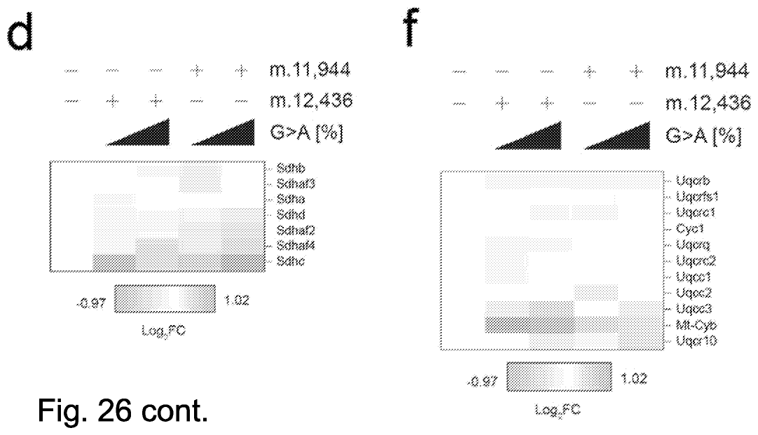

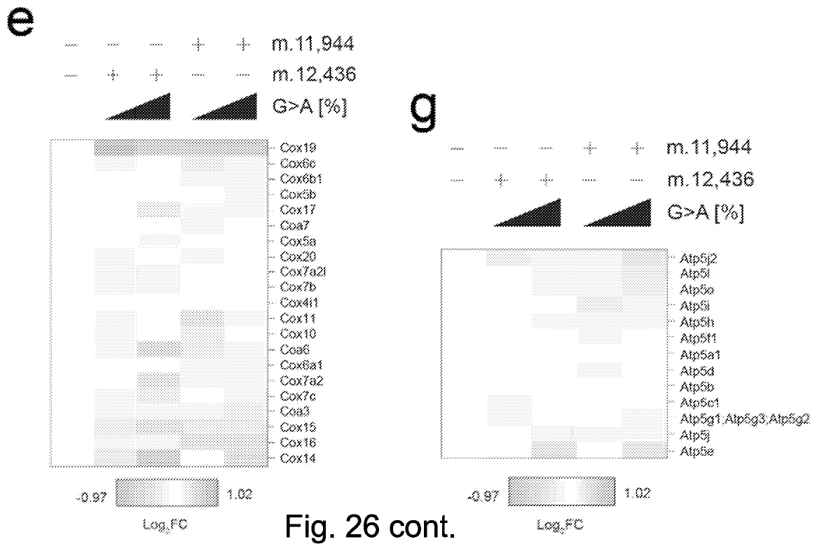

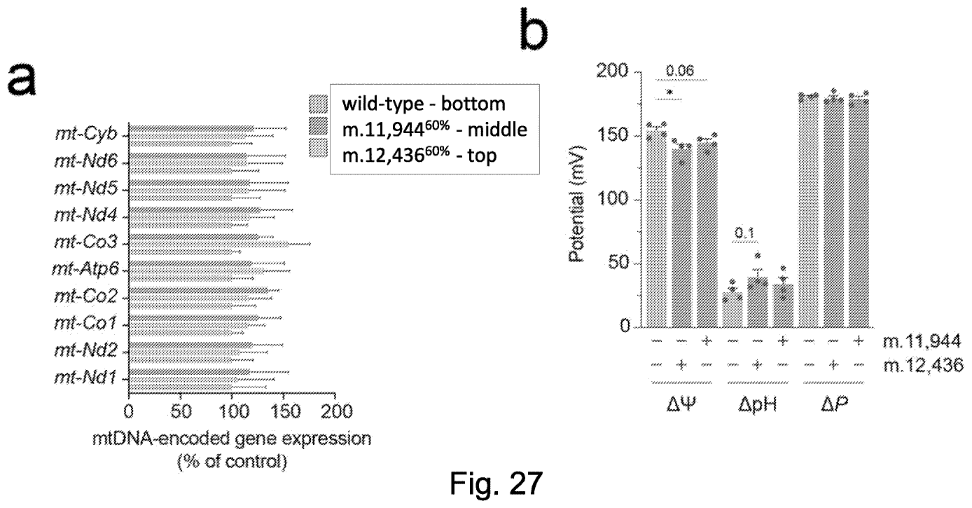

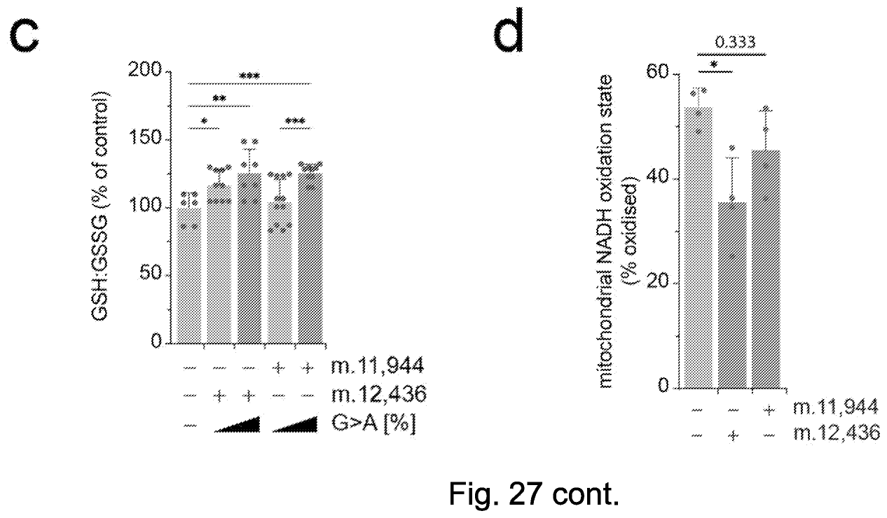

The inventors induced premature stop codons at tryptophan (TGA) codons within mouse mt-Nd5, analogous to hotspot mutations found in the human MT-ND5 gene in tumours¹ (FIG. 21A-C). TALE-DdCBE G1397/G1333 candidates, bearing nuclear export signals, targeting 20 m.12,436G>A and m.11,944G>A sites were synthesised and screened in mouse B78-D14 amelanotic melanoma cells (B.16 derivative, Cdkn2a null)⁹ to identify efficient pairs (FIG. 1D). Expression of functional pairs (FIG. 25A) resulted in isogenic cell populations bearing ~40% or ~60% mutation heteroplasmy of m.12,436 G>A or m.11,944 G>A truncating mutations following either a single transfection or four consecutive transfections (referred to as m.12,436^{40%}, m.12,436^{60%}, m.11,944^{40%} and m.11,944^{60%} respectively) (FIG. 21E) with limited off-target mutation (FIG. 25B). The resulting stable, isogenic cell lines demonstrated a heteroplasmy-dependent decrease in expression of complex I subunit Ndufb8 without impact on other respiratory chain components (FIG. 21F). This was supported by Tandem Mass Tagging (TMT)-based mass spectrometry proteomics (FIG. 26) and blue native PAGE analysis of the m.12,436^{60%} and m.11,944^{61%} cell lines (FIG. 21G), confirming that individual complex I subunit abundance, in addition to the proportion of fully assembled complex 1, is decreased without substantial impact on other components of the OXPHOS system. In-gel activity assays of complex I and complex II activity further support this finding (FIG. 21G). mtDNA copy number was not impacted by mutation incidence or heteroplasmy level (FIG. 21H) and mt-Nd5 transcript level was unchanged in m.12,436^{60%} and m.11,944^{60%} mutant cells compared with controls, consistent with lack of nonsense-mediated decay in mammalian mitochondria (FIG. 27A). Interestingly, none of the heteroplasmic cells exhibited significant decreases in oxygen consumption (FIG. 21I), adenylate energy charge state (FIG. 21J) or cell proliferation (FIG. 21K). However, a ~10 mV decrease in the electrical component of the mitochondrial proton motive force, Δ^{ψ}, coupled to a commensurate trend towards ~10 mV increases in the chemical component, ΔpH, resulting in an unchanged total protonmotive force, ΔP, was detected (FIG. 27B). The NAD+:NADH ratio was significantly impacted in mutant cells (FIG. 21L), which was also reflected in reduced:oxidised glutathione (GSH: GSSG) ratios (FIG. 27C). The effect on cellular redox poise was further determined in m.12,436^{60%} and m.11,944^{60%} cells using NAD(P)H fluorescence (FIG. 27D). Taken 10 together, these data demonstrate that truncating mutations in mt-Nd5 exert heteroplasmy-dependent effects on the abundance of complex 1. In turn, partial loss of complex I disrupts cellular redox balance, without significantly impacting cellular energy homeostasis, oxygen consumption or proliferation.

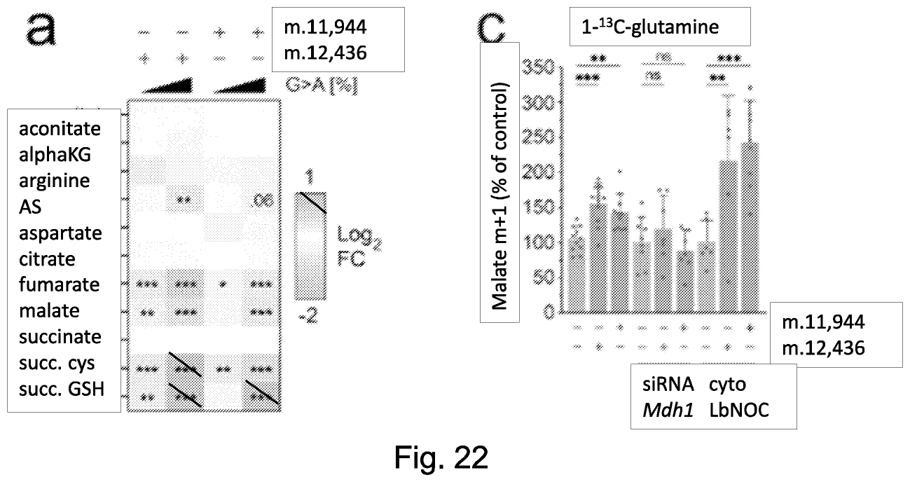

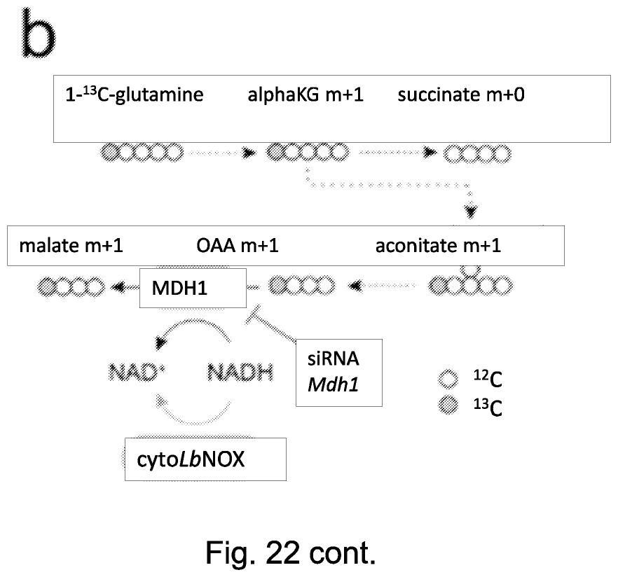

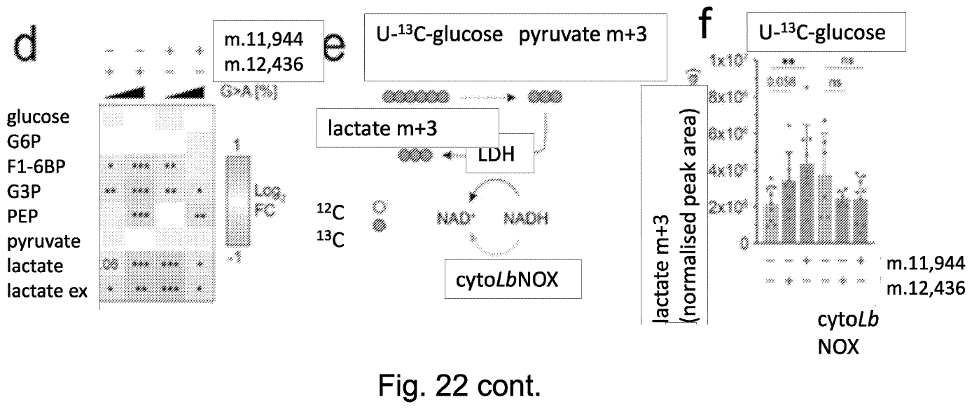

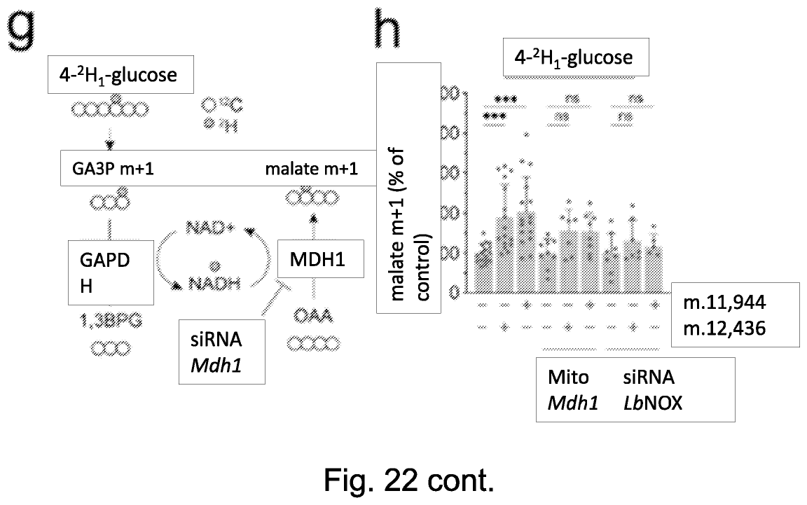

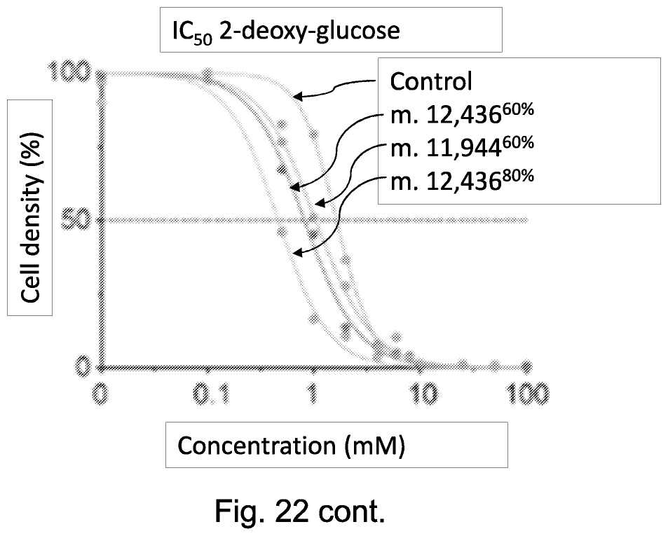

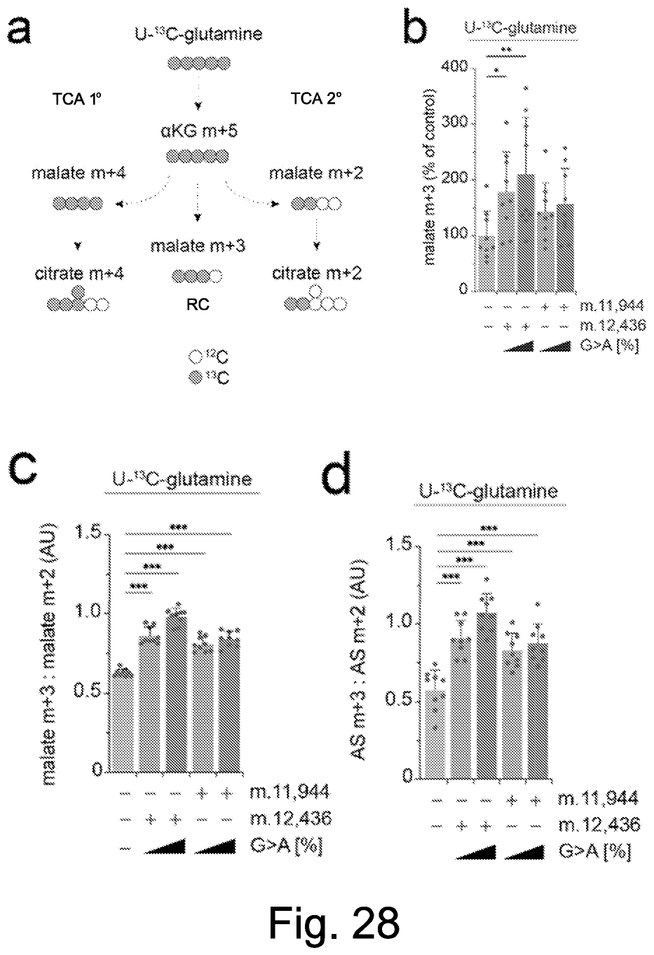

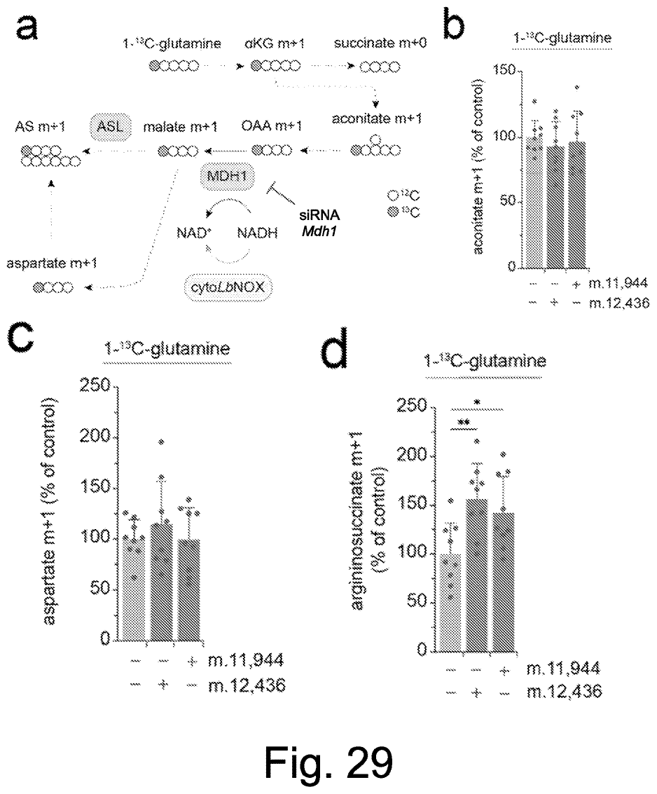

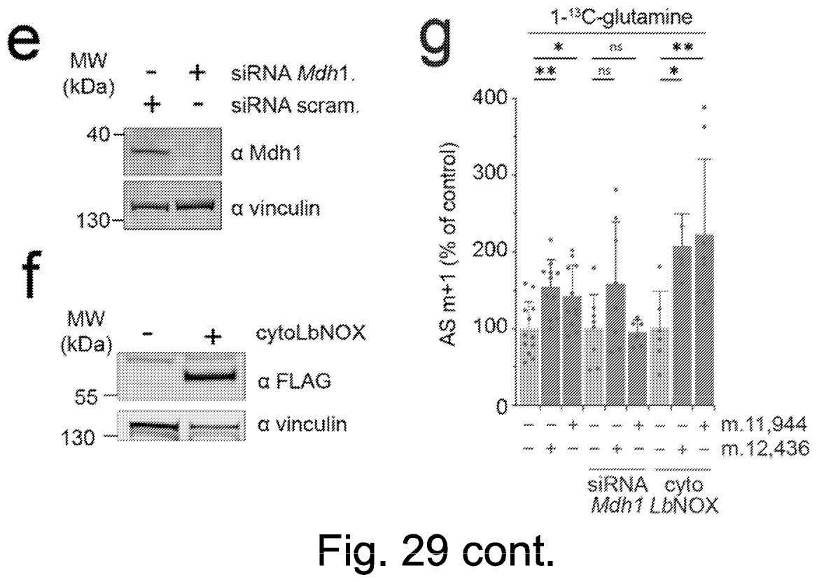

Unlabelled metabolomic measurements from m.12,436^{60%} and m.11,944^{60%} cells revealed consistent differences in metabolite abundance in these cells relative to control (FIG. 5), with notable increases in the steady-state abundance of malate, lactate, fumarate, argininosuccinate (AS) and the metabolically terminal fumarate adducts succinylcysteine and succinicGSH (FIG. 22A). Heteroplasmy-dependent increases in abundance of lactate and malate in the context of constant succinate in mutant cells suggested that the flow of electrons into mitochondria through the malate-aspartate shuttle (MAS) might be impacted by changes to the redox state of the cell. To study this the inventors first measured the contributions of glutamine-derived carbon to tricarboxylic acid (TCA) cycle metabolites using U-¹³C-glutamine isotope tracing (FIG. 28A). This indicated increased abundance of malate from cytosolic oxaloacetate (OAA), derived from citrate via ATP citrate lyase, as determined by the abundance of malate m+3 and the ratio of malate m+3:m+2, which demonstrated a significant, heteroplasmy-dependent increase relative to control (FIG. 28B, C), with a similar pattern of m+3:m+2 labelling observed for urea cycle metabolite AS (FIG. 28D). The inventors then traced the metabolic fate of carbon from 1-¹³C-glutamine, which exclusively labels metabolites derived from reductive carboxylation (RC) of glutamine (FIG. 22B, FIG. 29A). This revealed that the increased abundance of malate m+1 occurred at the level of MDH1 (FIG. 22C), but was not apparent in downstream or upstream metabolites aconitate and aspartate (FIG. 29B, C), with the m+1 labelling pattern of AS again matching that of malate (FIG. 29D). The increased abundance of malate m+1 and AS+1 was sensitive to siRNA mediated depletion of Mdh1 but not expression of cytosolically targeted LbNOX (cytoLbNOX), a water-forming NADH oxidase¹⁰ (FIG. 22C, FIG. 29E-G), indicating that increases in malate abundance occur at least partially in the cytosol via MDH1, but are not directly due to gross alteration in cytosolic NAD+:NADH redox poise.

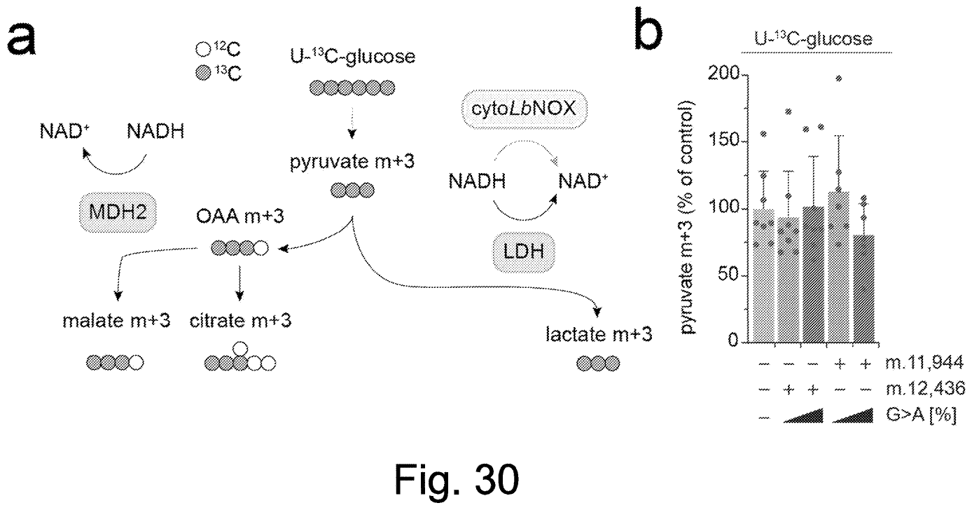

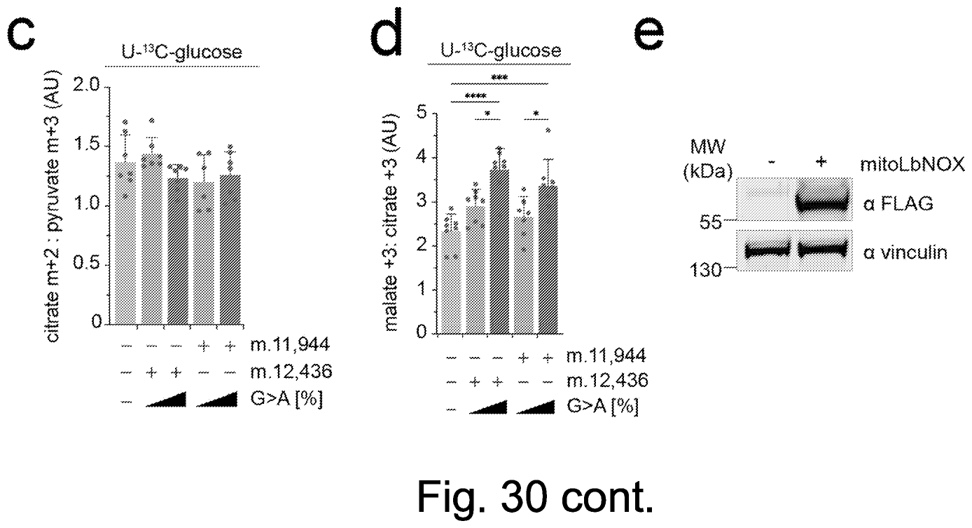

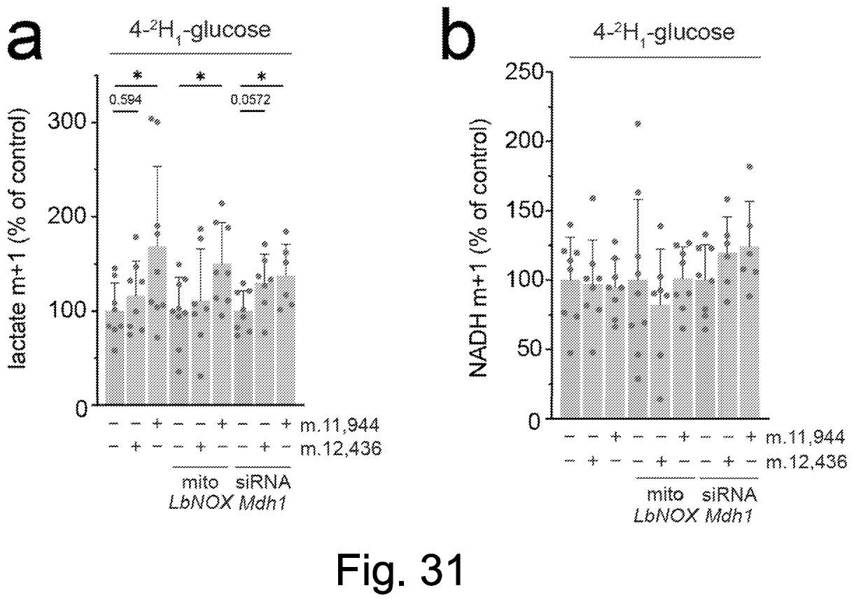

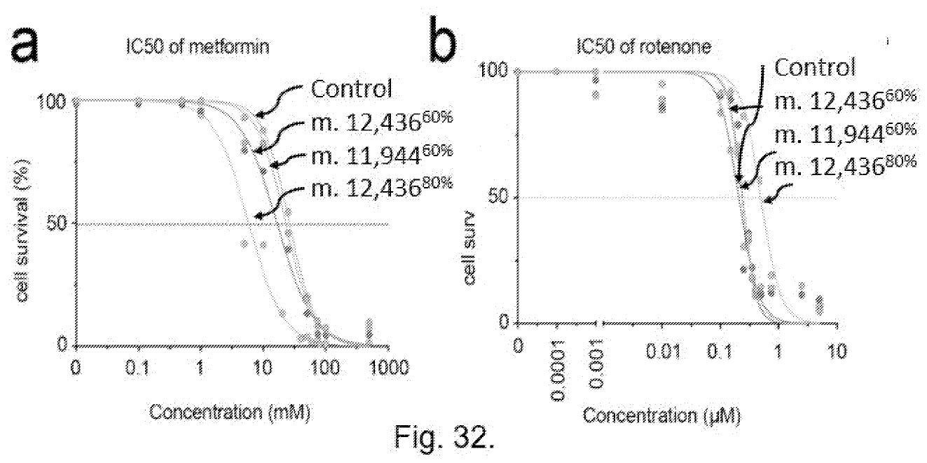

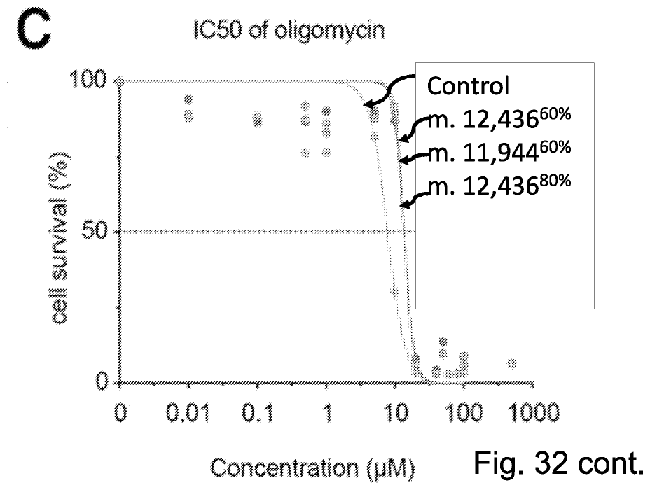

Elevated cellular and extracellular lactate, alongside increased abundance of several glycolytic intermediates (FIG. 22D) suggested utilisation of pyruvate as an electron acceptor to rebalance NAD+:NADH via lactate dehydrogenase (LDH). Using U-¹³C-glucose tracing (FIG. 22E) the inventors observed increased abundance of lactate m+3 in m.12,436^{60%} and m.11,944^{60%} cells that was abolished by cytoLbNOX expression (FIG. 22F, FIG. 30A). The increase in lactate m+3 did not alter pyruvate m+3 levels (FIG. 30B), or the entry of glucose-derived carbon into the TCA cycle via pyruvate dehydrogenase (PDH) determined by the ratio of citrate m+2:pyruvate m+3 (FIG. 30C). However, the fate of carbon entering the TCA cycle via pyruvate carboxylase (PC) was substantially altered, with a malate m+3:citrate m+3 ratio indicative of MDH2 reversal (FIG. 30D). Coupling of the MAS with glycolysis is a topic of recent interest, with several reports linking mitochondrial dysfunction with NADH shuttling between GAPDH and MDH1/LDH^{11,12}. Using 4-²H₁-glucose isotope tracing (FIG. 22G) the inventors observed an increase in abundance of malate m+1 in m.12,436^{60%} and m.11,944^{60%} cells, with a similar trend in lactate m+1 abundance, that was sensitive to mitoLbNOX treatment and siRNA mediated depletion of Mdh1(FIG. 22H, FIG. 31A, B), supporting the notion that the NAD+:NADH imbalance resulting from partial loss of complex I supports enhanced glycolytic flux through coupling cytosolic portions of the MAS with glycolysis. In turn, this increased glycolytic flux rendered m.12,436^{60%} (IC₅₀=0.81 mM±0.064 mM) and m.11,944^{60%} cells (IC₅₀=1.04 mM±0.040 mM) more sensitive to the competitive phosphoglucoisomerase inhibitor 2-deoxyglucose (2-DG) compared with wild-type cells (IC₅₀=1.62 mM±0.063 mM) (FIG. 22I), a sensitivity that was further enhanced in a m.12,436^{80%} model (IC₅₀=0.46 mM±0.080 mM). m.12,436^{60%}, m.12,436^{80%} and m.11,944^{60%} cells also demonstrated enhanced sensitivity to the low affinity complex I inhibitor metformin relative to wild-type (FIG. 32A). The 60% mutants were not differentially sensitive to potent complex I inhibitor rotenone, although interestingly the m.12,436^{80%} demonstrated resistance compared to wild type (FIG. 32B). None of the mutants demonstrated differential sensitivity to complex V inhibitor, oligomycin (FIG. 32C). Taken together, these data demonstrate that truncating mutations in mt-Nd5 of complex I induce a Warburg-like metabolic state through redox imbalance, not energetic crisis. This influences both cytosolic and mitochondrial components of the MAS, increasing glycolytic flux, enhancing sensitivity to inhibition of this adaptive metabolic strategy and producing elevated levels of characteristic terminal fumarate adducts succinicGSH and succinylcysteine.

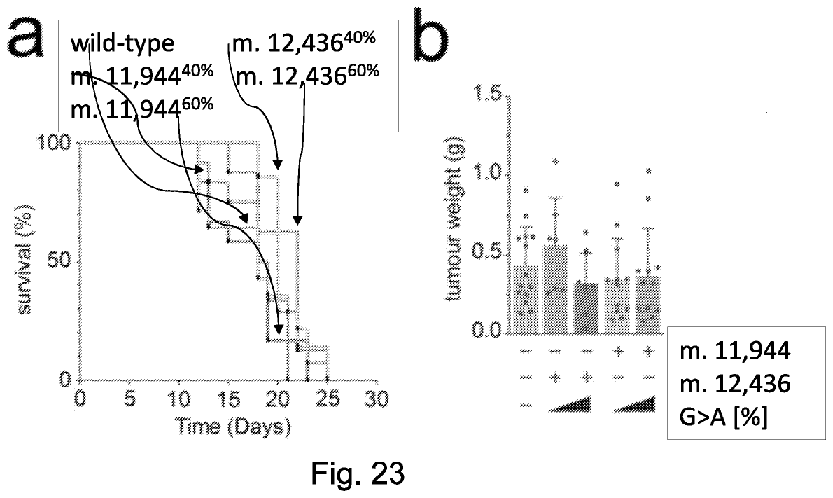

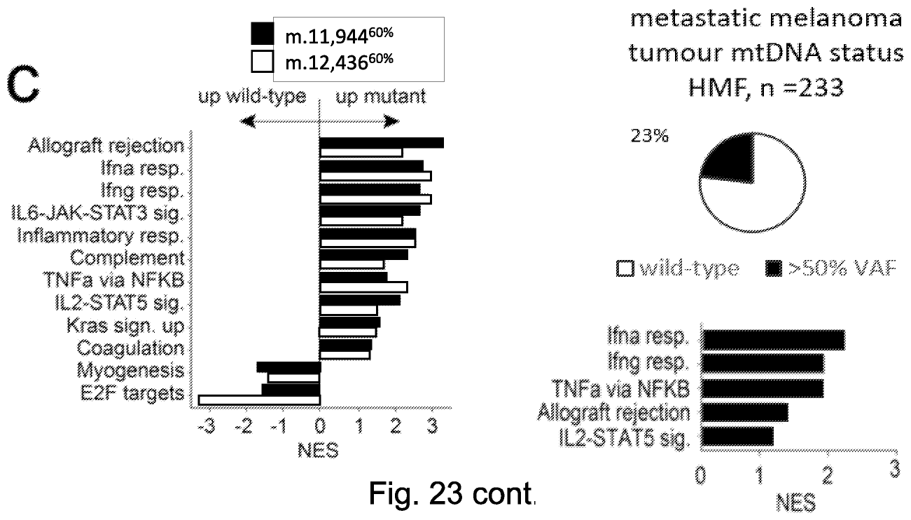

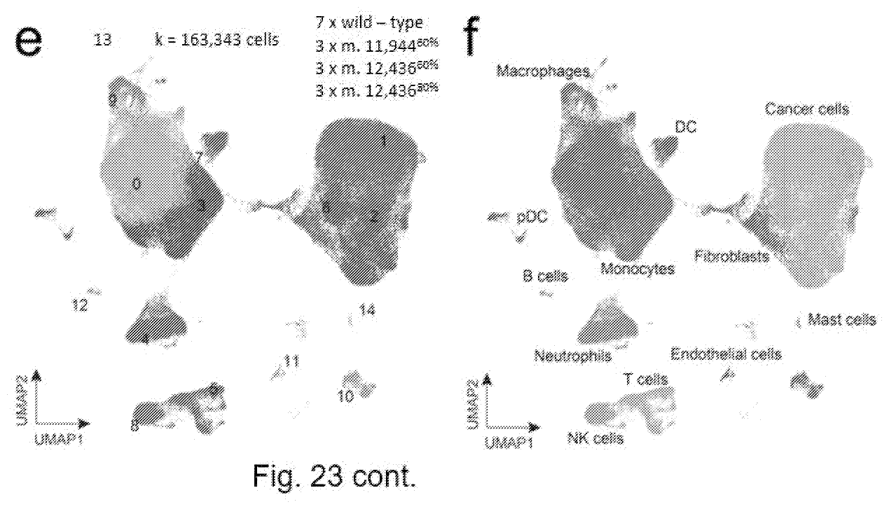

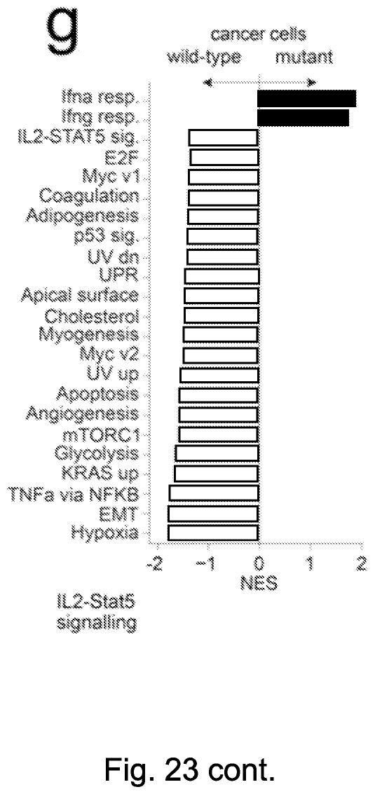

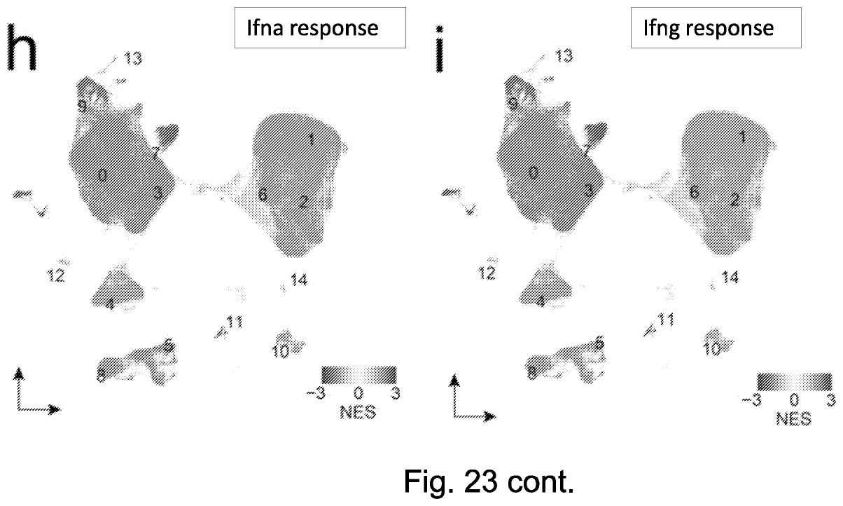

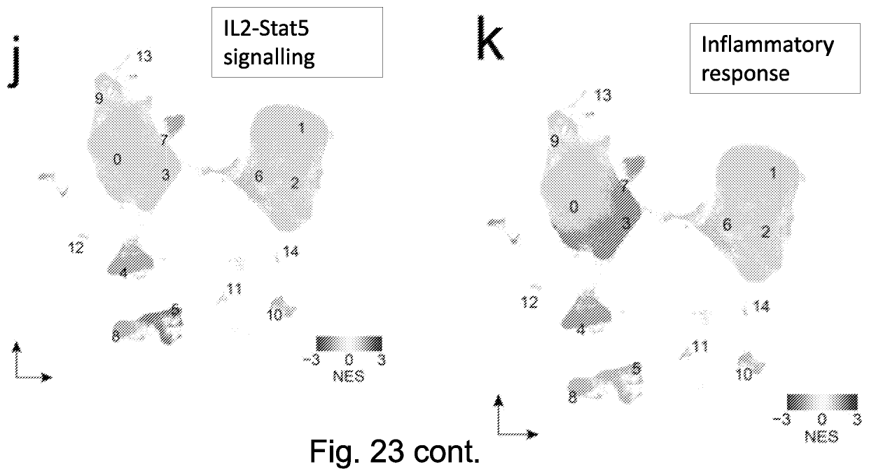

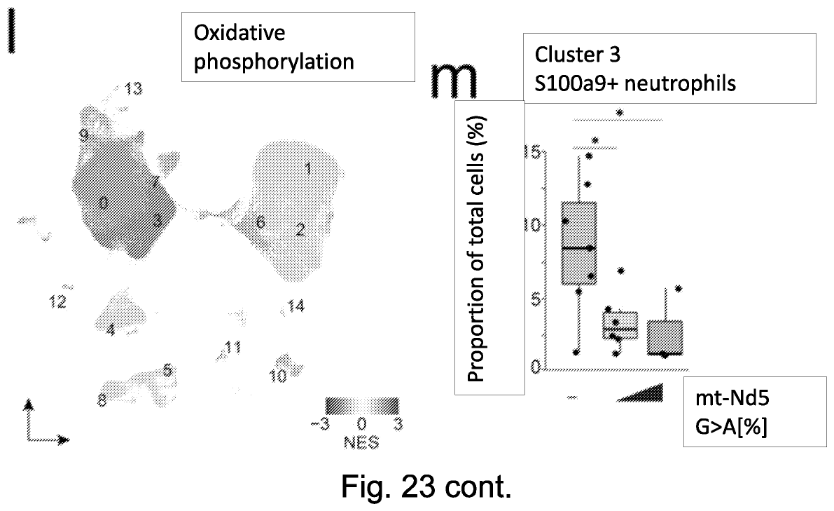

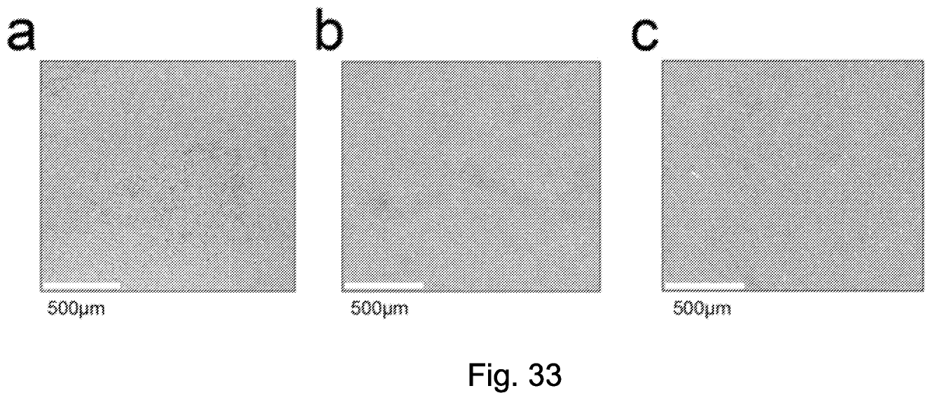

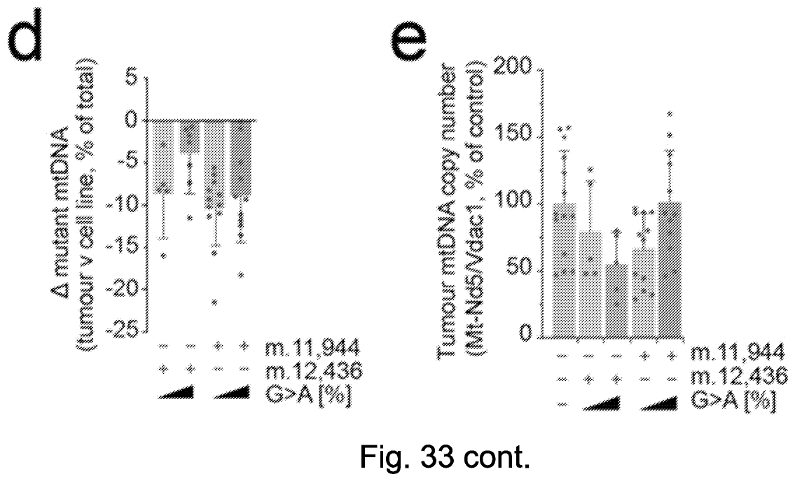

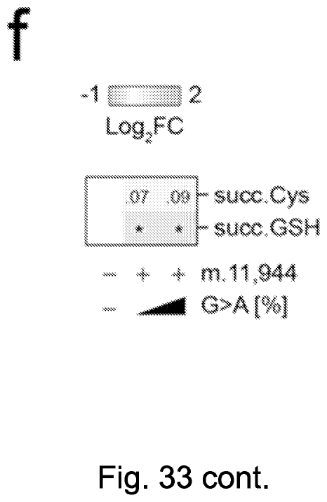

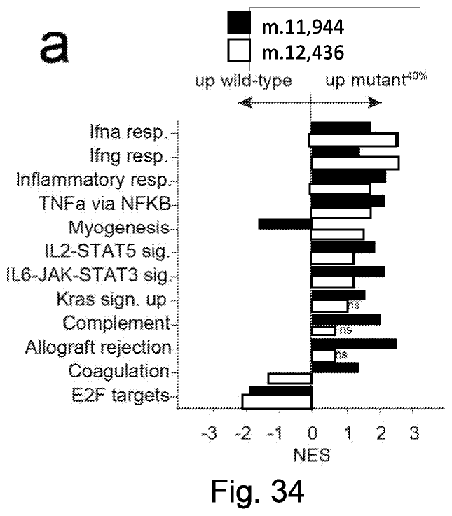

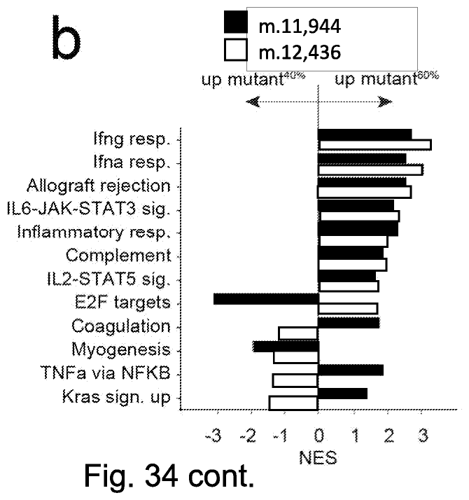

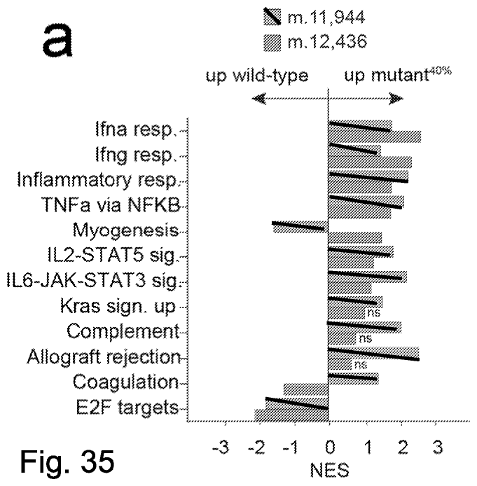

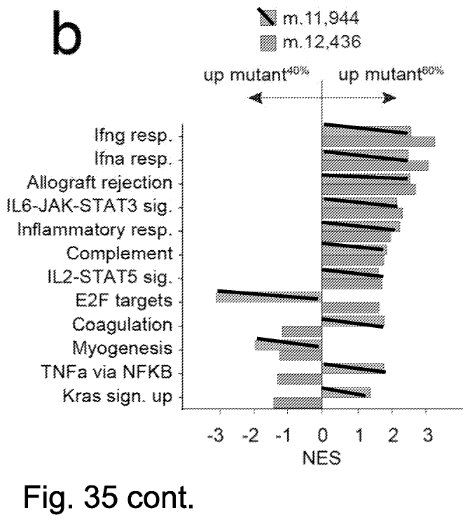

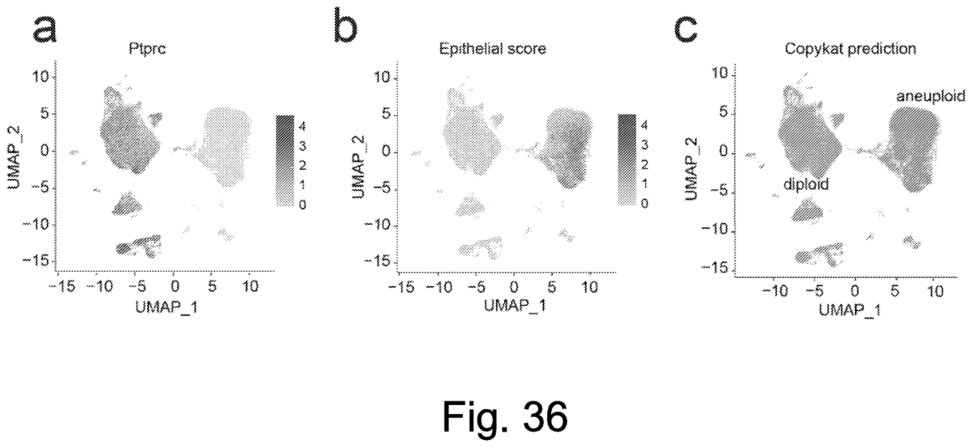

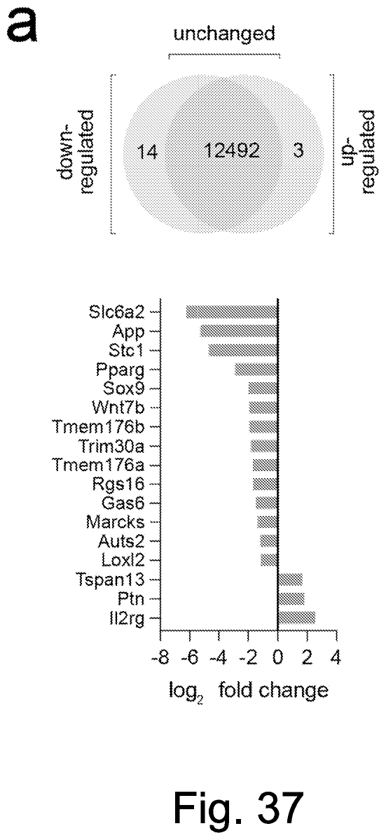

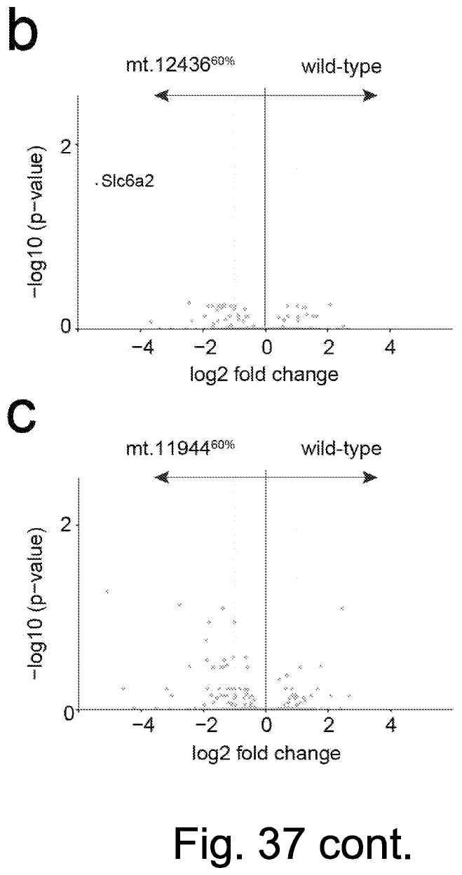

Having established specific changes in redox metabolism driven by truncating mutations in complex I, the inventors next sought to determine the impact of these metabolic alterations in tumour biology. Syngeneic allografts of m.11,944 G>A cells, m.12,436 G>A cells and wild-type controls were performed subcutaneously in immunocompetent C57/Bl6 mice, establishing tumours in 100% of engraftments. All tumours grew at a rate that reached similar humane endpoints (FIG. 23A) with similar weights and macroscopic histological features (FIG. 23B, FIG. 33A-C). Bulk measurements of tumour heteroplasmy revealed a subtle, comparable decrease in heteroplasmy of ~10% between engrafted cells and resulting tumours, likely reflecting stroma and immune cell infiltrate (FIG. 33D), with no consistent change in mtDNA copy number detected at bulk level (FIG. 33E). Measurements of metabolites from m.11,944^{60%} mutant and control tumours revealed elevated abundance of terminal fumarate adducts succinicGSH and succinylcysteine, characteristic of the metabolic rewiring observed in vitro (FIG. 33F). These markers of a consistently altered tumour metabolic profile were coupled to divergent transcriptional signatures between control and mutant tumours (FIG. 23C), with several signatures of altered immune infiltrate and signalling being significantly elevated in mutant tumours compared with controls, notably allograft rejection, interferon gamma (Ifng) and interferon alpha (Ifna) responses. Higher heteroplasmies correlated to increased signal in the same gene sets (FIG. 34) suggesting a heteroplasmy dose-dependent anti-tumour immune response. To benchmark these findings against human data, the inventors took the Hartwig Medical Foundation (HMF) metastatic melanoma cohort and stratified this by pathogenic mtDNA mutation status into wild-type and >50% variant allele frequency (VAF) groups (see Methods). This yielded a set of 355 tumour samples (272 wildtype, 83>50% VAF), with 233 having transcriptional profiles. GSEA analysis revealed consistent transcriptional phenotypes between patient tumours bearing high heteroplasmy pathogenic mtDNA mutations and those identified in our model systems (FIG. 23D), supporting the observation. To further dissect these effects the inventors employed whole tumour single cell RNA sequencing (scRNAseq) across seven control, three m.12,436^{60%}, three m.11,944^{60%} and three m.12,436^{80%} tumours, resulting in 163,343 single cell transcriptomes. Cells were clustered using Seurat and cellRanger, with preliminary cell ID determined by scType (see Methods) (FIG. 35E,F). Malignant cells were assigned on the basis of: i) low or nil Ptprc (CD45) expression; ii) high epithelial score¹³; iii) aneuploidy determined by copykat analysis¹⁴ (FIG. 35). Consistent with bulk tumour transcriptional profiles, GSEA in malignant cells revealed increased Ifna and Ifng signatures coupled to decreased glycolysis signatures in high heteroplasmy tumours (FIG. 23G), which is not observed in vitro prior to implantation (FIG. 36). Downstream regulation of primary metabolic and subsequent immune signalling on malignant cells are also reflected in altered nutrient sensing by mTORC1, transcriptional control of metabolic genes by myc, and TNFa signalling (FIG. 23G). GSEA in non-malignant cell clusters revealed similar tumour-wide changes in transcriptional phenotype, with increased Ifna, Ifng, inflammatory response and IL2-Stat5 signalling again observed (FIG. 23H-K). These indicators of a broad anti-tumour immune response were accompanied by decreased neutrophil residency (FIG. 3L) and altered monocyte maturation (FIG. 37A, B), with a switch in neutrophil metabolic state indicated by increased OXPHOS gene expression (FIG. 23M). Further genesets typical of an augmented anti-tumour response, such as allograft rejection, were also elevated alongside a biphasic trend in proportions of tumour resident natural killer and CD4+ T cells (FIG. 37C-E). Taken together these data demonstrate that, in a heteroplasmy-dependent fashion, mt-Nd5 mutation is sufficient to remodel the tumour microenvironment (TME) and promote an anti-tumour immune response.

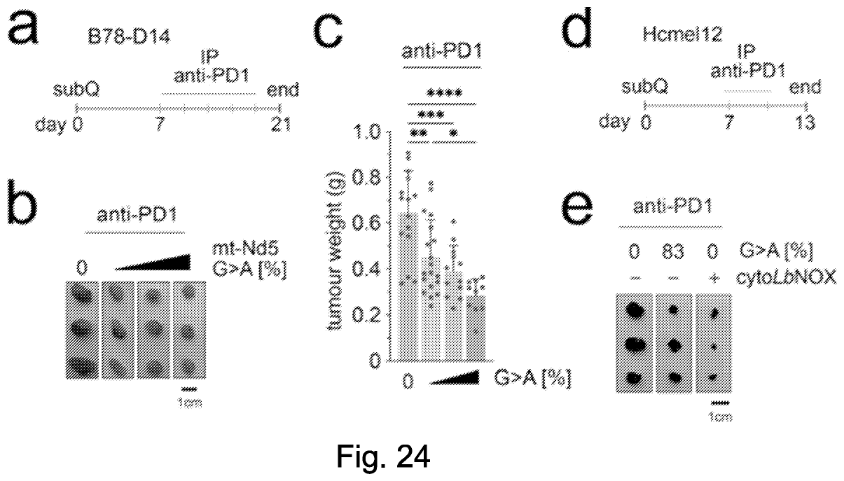

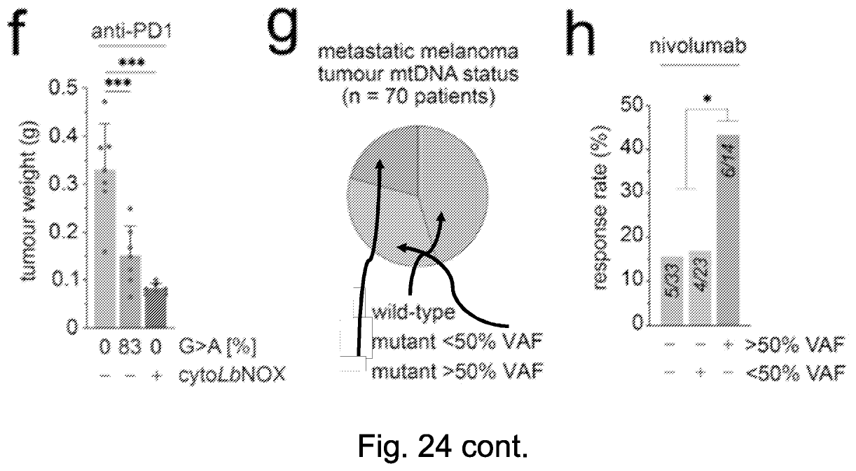

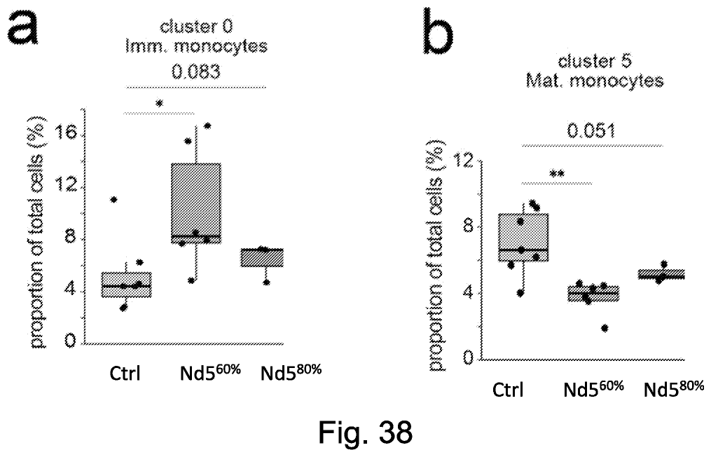

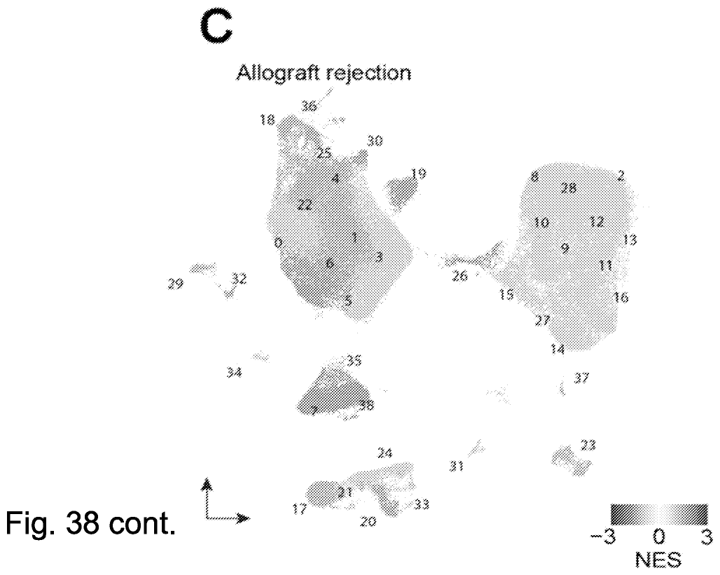

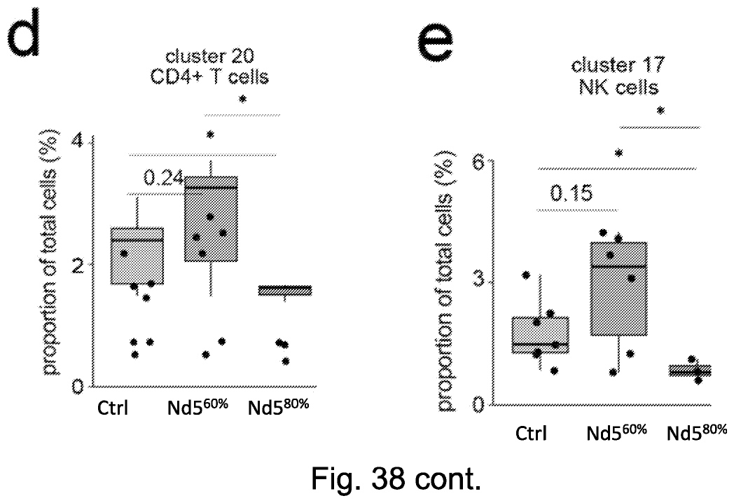

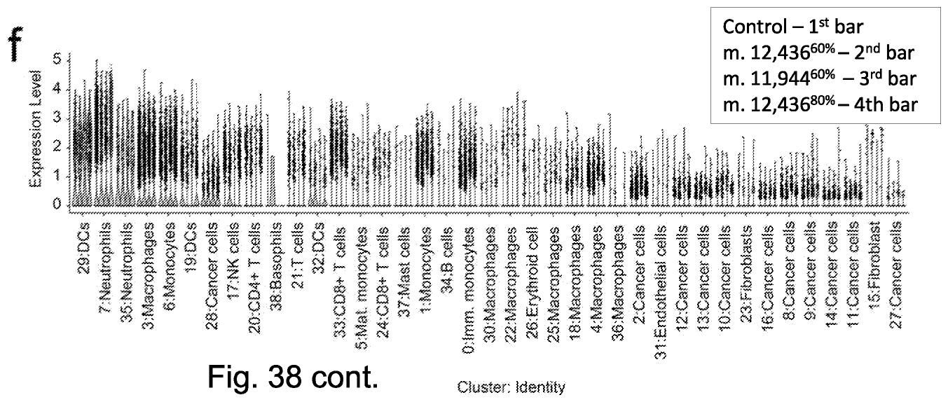

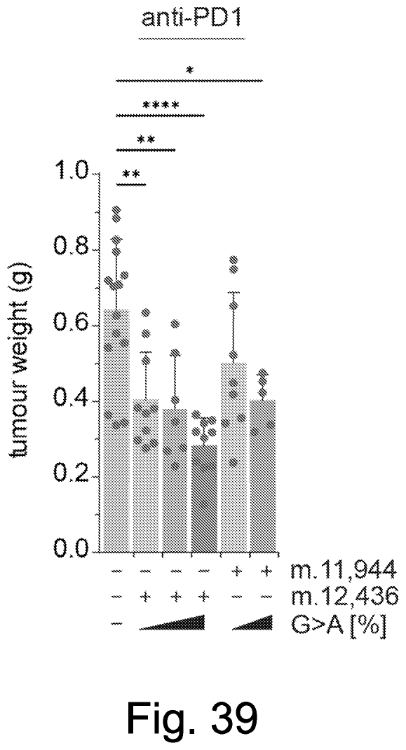

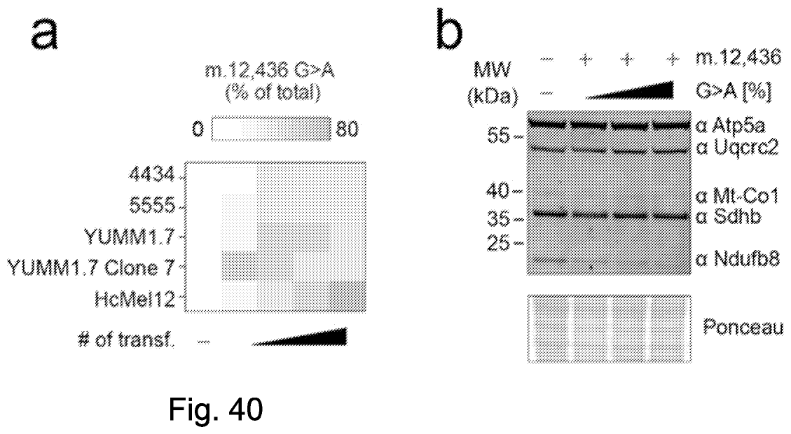

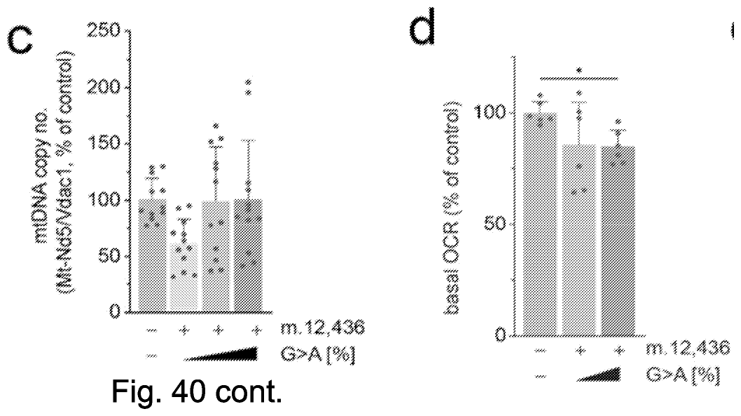

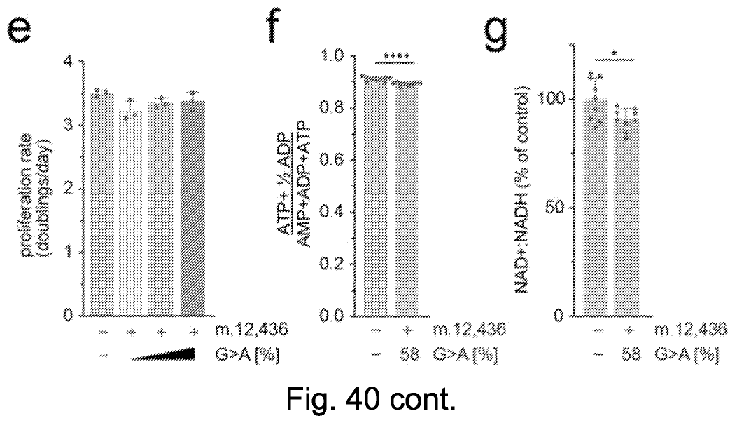

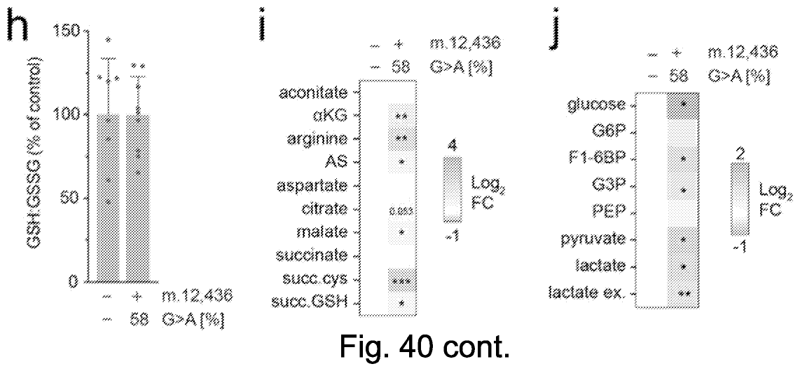

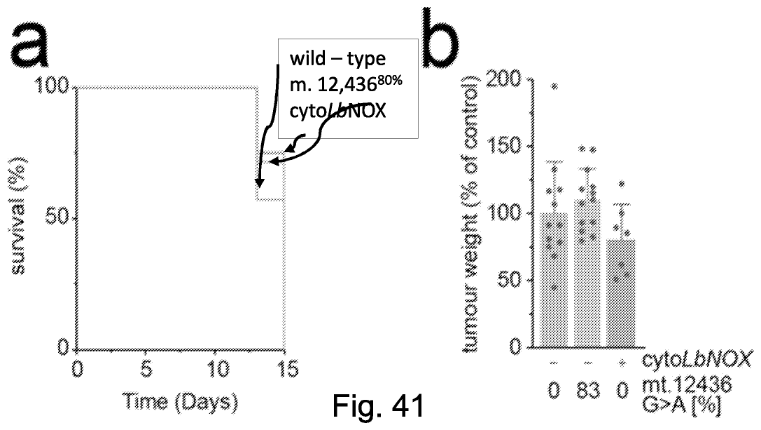

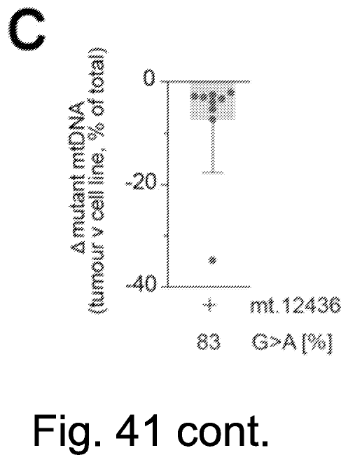

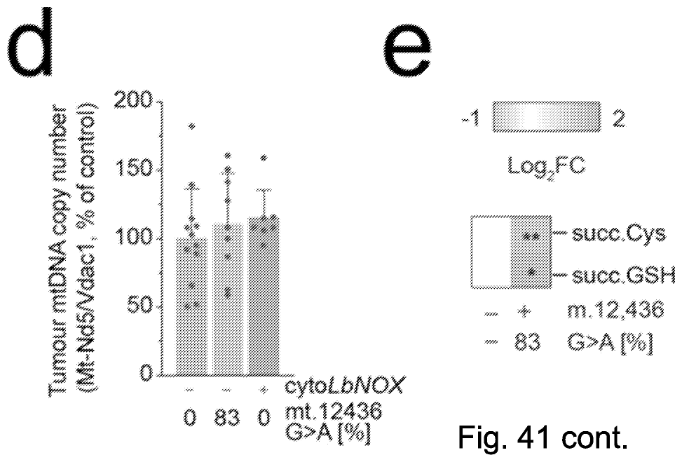

Treatment of malignant melanoma can include immune checkpoint blockade (ICB) with monoclonal antibodies (mAbs) against T and B-cell expressed immune checkpoint receptor PD1, blocking PD-L½ binding to limit tumour-induced immune tolerance. However, the effectiveness of anti-PD1 treatments, and ICB response in melanoma patients more broadly is bimodal, with a substantial proportion of patients not responding to treatment while experiencing a poor morbidity profile. Limited efficacy of ICB has been linked to immunosuppressive tumour-associated neutrophils previously¹⁵ therefore the inventors reasoned that mt-Nd5 mutant tumours could demonstrate differential sensitivity to ICB, even in an aggressive model of poorly immunogenic melanoma such as B78-D14. Additionally, depleted neutrophil populations in mt-Nd5 mutant tumours also demonstrated the greatest PD-L1 expression (FIG. 37F). To test this the inventors performed further subcutaneous syngeneic allografts of m.12,436^{40%}, m.12,436^{60%}, m.12,436^{80%}, m.11,944^{40%}, m.11,944^{60%} and wild-type tumours in immunocompetent animals. Tumours grew untreated for 7 days post-graft and animals were dosed with a regimen of intraperitoneal anti-PD1 mAb every 3 days until conclusion of the experiment (FIG. 24A). A heteroplasmy-defined decrease in tumour 25 weight at endpoint was observed across the mtDNA mutant tumours, with higher mutant heteroplasmies exhibiting greater response to treatment (FIG. 2 4B,C, FIG. 38), consistent with increased sensitivity of mtDNA mutant tumours to immunotherapy. To verify these data the inventors attempted to produce further independent models of aggressive, poorly immunogenic mouse melanoma (FIG. 39A). This yielded Hcmel12 (Hgf, Cdk4^{R24C})¹⁶ cells engineered to bear >80% m.12,436 G>A mutation, demonstrating consistent cellular and metabolic phenotypes with B78-D14 (FIG. 39B-J). Hcmel12 m.12,436^{80%} and wild-type Hcmel12 cells were engrafted into mice with a similar experimental workflow as previously (FIG. 24D). When untreated, Hcmel12 m.12,436⁸⁰ and wild-type tumours demonstrate comparable time to endpoint and tumour weight at endpoint (FIG. 40A, B). Changes in bulk heteroplasmy, copy number and tumour metabolism were also similar to those of B78-D14 tumours (FIG. 40C-D). Moreover, when anti-PD1 treatment was administered, a mtDNA mutation-dependent response was observed in Hcmel12 of similar magnitude to that seen in B78-D14 (FIG. 24 E,F). To dissect the enhanced ICB response into metabolic v.s. non-metabolic effects of mtDNA mutation, the inventors modified wild-type Hcmel12 cells to constitutively express cytoLbNOX, which reproduces key elements of the cell-extrinsic, mutant Mt-Nd5-associated metabolic phenotype, notably glucose uptake and lactate release (FIG. 41). When grafted into mice, Hcmel12 cytoLbNOX tumours demonstrated comparable time to endpoint and tumour weight at endpoint as wild-type or Mt-Nd5 mutant tumours (FIG. 40A,B). When challenged with anti-PD1 treatment, Hcmel cytoLbNOX tumours recapitulate the response of Hcmel mt-Nd5 m.12,436⁸⁰′ tumours, indicating that specific changes in redox metabolism associated with mtDNA mutation are sufficient to sensitise the tumour to ICB (FIG. 24E,F). To benchmark these findings from mice against real world clinical data, the inventors re-analysed a previously reported, well-characterised cohort of majority treatment-naive metastatic melanoma patients given a dosing regimen of the anti-PD1 mAb nivolumab¹⁷. By identifying mtDNA mutant cancers and stratifying this patient cohort solely on the basis of cancer mtDNA mutation status (FIG. 24G) the 70 patients in this cohort were divided into three groups: mtDNA wild-type (33), <50% VAF (23), and >50% VAF (14). The cancer mtDNA mutation status-naive cohort response rate was 22% for partial or complete responses to nivolumab, however the rate of response for >50% mtDNA mutation VAF cancers was 2.6-fold greater than wild-type or <50% VAF cancer (FIG. 24H), recapitulating our laboratory findings in patients.

These data confirm that somatic mtDNA mutations, commonly observed in human tumours, can exert direct effects on cancer cell metabolic phenotypes. In contrast with clinically presented germline mtDNA mutations,⁶ tumour mtDNA mutations are able to exert these effects at a comparably low heteroplasmic burden and without negatively impacting oxygen consumption or energy homeostasis. The direct link observed between redox perturbations and enhanced glycolytic flux subtly alters our view of mtDNA mutation, to an adaptive gain of function rather than exclusively loss of function event, and the discovery that mtDNA mutations can underpin aerobic glycolysis warrants further assessment of the relationship between classical Warburg metabolism¹⁸ and mtDNA mutation status.

Beyond cancer cell intrinsic effects, the data here reveal that a functional consequence of somatic mtDNA mutation in tumour biology is the remodelling of the TME, mediating therapeutic susceptibility to ICB. Truncating mutations to mtDNA, analogous to those described here, affect 10% of all cancers regardless of tissue lineage, with non-truncating, pathogenic mtDNA mutations presenting in a further 40-50% of all cancers. A broad influence over the anti-tumour immune response in these cancers might also be expected.

Beyond exploitation of mtDNA mutant tumour vulnerability, our data suggest that the ICB response-governing effects the inventors observe are principally metabolic in nature. Recreating such a metabolic state in mtDNA wild-type or ‘immune cold’ tumour types could therefore also be of benefit.

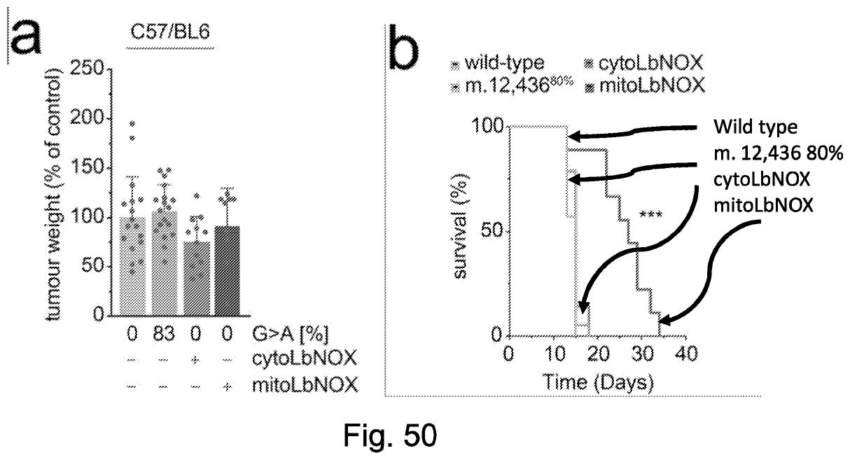

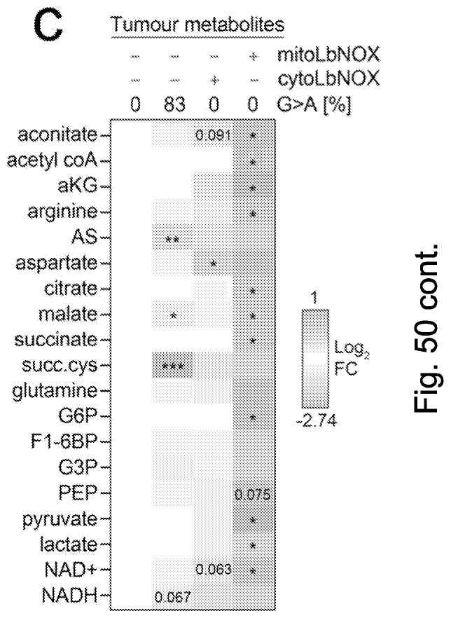

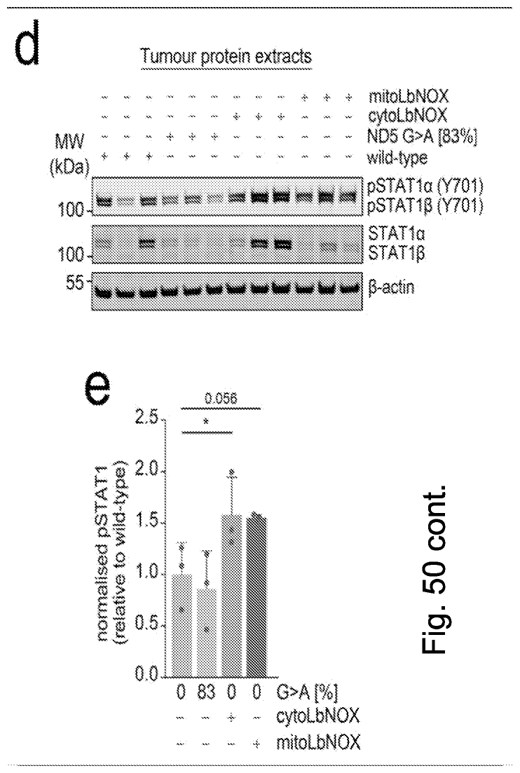

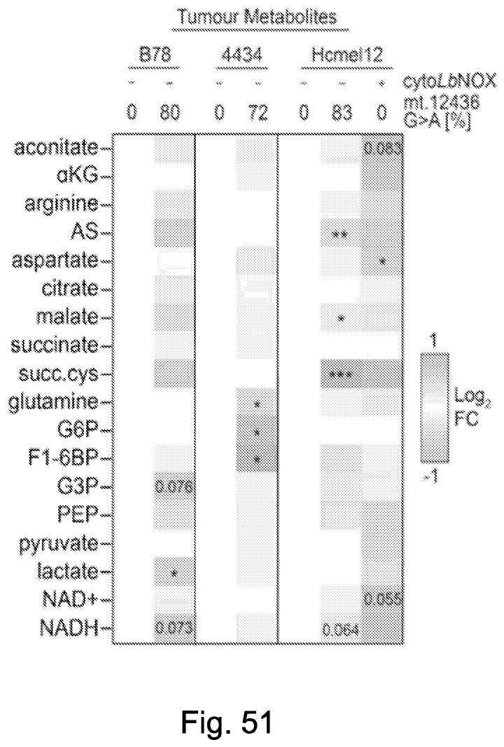

Furthermore, the inventors have shown that in tumours expressing mitoLbNOX there is in fact substantial elevation in the levels of pSTAT1 when compared to wildtype. When taken with the rest of the data shown in FIG. 50, this suggests that mitoLbNOX will exert a similar effect upon treatment with immunotherapy as cytoLbNOX, if not a more potent effect (as the growth of mitoLbNOX tumours in the untreated setting is substantially slower than wildtype in immunocompetent animals, where cytoLbNOX tumour growth is comparable to wild-type in the untreated, immunocompetent setting.

### Methods

### Maintenance, Transfection and FACS of Cell Lines

B78 melanoma cells (RRID:CVCL_8341) and Hcmel12 cells¹⁶ were maintained in DMEM containing GLUTAMAX™, 0.11 g/L sodium pyruvate, 4.5 g/L D-glucose (Life Technologies) and supplemented with 1% penicillin/streptomycin (P/S) (Life Technologies) and 10% FBS (Life Technologies). Cells were grown in incubators at 37° C. and 5% CO₂. Cells were transfected using Lipofectamine 3000 (Life Technologies) using a ratio of 5 μg DNA: 7.5 μl Lipofectamine 3000. Cells were sorted as outlined in¹⁹ and thereafter grown in the same base DMEM media supplemented with 20% FBS and 100 μg/mL of uridine (Sigma).

### Use of Animal Models

Animal experiments were carried out in accordance with the UK Animals (Scientific Procedures) Act 1986 (P72BA642F) and by adhering to the ARRIVE guidelines with approval from the local Animal Welfare and Ethical Review Board of the University of Glasgow. Mice were housed in conventional cages in an animal room at a controlled temperature (19-23° C.) and humidity (55±10%) under a 12 hr light/dark cycle. Experiments only used male C57BL/6 mice at ~8 weeks of age which were injected subcutaneously with either 2.5×10⁵ B78 cells or 1×10⁴ HcMel12 cells, both prepared in 1:1 RPMI (Life Technologies) and Matrigel (Merck). Mice were culled at an endpoint of 15 mm tumour measurement.

For immunotherapy experiments, mice were put on a dosing regimen of 200 μg of anti-PD1 given intraperitoneally twice a week. The first dose was given 7 days post-injection and all mice were sacrificed at 21 or 13 days post-injection for B78 or HcMel12 cells respectively.

### Construction of DdCBE Plasmids

TALEs targeting mt.12,436 and mt.11,944 were designed with advice from Beverly Mok and David Liu (Broad Institute, USA). TALEs were synthesised (ThermoFisher GeneArt) as illustrated in FIG. 1A with the left TALEs being cloned into pcDNA3.1(−)_mCherry¹⁹ and the right into pTracer CMV/Bsd¹⁹, allowing for the co-expression of mCherry and GFP respectively.

### Pyrosequencinq Assay

DNA was extracted from cell pellets using the DNeasy Blood & Tissue Kit (Qiagen) as per the manufacturer's instructions. PCR was then performed using the PyroMark PCR Mix (Qiagen) for 50 cycles with an annealing temperature of 50° C. and an extension time of 30 sec. PCR products were run on the PyroMark Q48 Autoprep (Qiagen) as per the manufacturer's instructions.

### PCR Primers for Mt.12,436

Forward: 5′-ATATTCTCCAACAACAACG-3′
Reverse: 5′-biotin-GTTATTATTAGTCGTGAGG-3′

### PCR Primers for Mt.11,944

Forward: 5′-CTTCATTATTAGCCTCTTAC-3′
Reverse: 5′-biotin-GTCTGAGTGTATATATCATG-3′

### PCR Primers for Mt.12,436

5′-TTGGCCTCCACCCAT-3′

### Sequencing Primer for Mt.11,944

5′-TAATTACAACCTGGCACT-3′

### Protein Extraction and Measurement

Cell pellets were lysed in RIPA buffer (Life Technologies) supplemented with cOmplete Mini Tablets and cOmplete Mini Protease Inhibitor Tablets (Roche). Samples were incubated on ice for 20 mins and then spun at 14,000 g for 20 mins. The isolated supernatant containing total cellular protein was then quantified using a DC Protein Assay (Bio-Rad Laboratories) performed as per the manufacturer's instructions.

### Immunoblotting

To detect protein via western blotting 60 μg of protein was resolved on SDS-PAGE 4-12% Bis-Tris Bolt gels (Life Technologies). Protein was transferred onto a nitrocellulose membrane using a Mini Trans-Bolt Cell (Bio-Rad Laboratories). Membranes were then stained with Ponceau S Staining Solution (Life Technologies) to measure loading before overnight incubation with the primary antibody prepared in 5% milk in 1×TBST. Imaging was performed using the Odyssey DLx Imaging system (Licor).

### Antibodies:

Total OXPHOS Rodent WB Antibody Cocktail (1:800, ab110413, Abcam)

Monoclonal Anti-FLAG® M2 antibody (1:1000, F1804, Sigma)

Recombinant anti-vinculin antibody (1:10,000, ab129002, Abcam)

### Mitochondrial Isolation

Cells were grown in Falcon Cell Culture 5-layer flasks (Scientific Laboratory Supplies) and grown to near 100% confluency. Cells were then harvested and mitochondria were extracted as described in²⁰.

### Blue-Native PAGE

Isolated mitochondria were solubilized in 1× NativePage Sample Buffer supplemented with 1% Digitonin (Life Technologies). Samples were incubated on ice for 10 min and then centrifuged at 20,000 g for 30 min at 4° C. Supernatants were isolated and total extracted protein quantified using the DC Protein Assay (Bio-Rad Laboratories). Samples were prepared and run on NativePage 4-12% Bis-Tris gels as per the manufacturer's instructions (Life Technologies). For immunoblotting, samples were transferred onto PVDF membranes using Mini Trans-Bolt Cell (Bio-Rad Laboratories). Subsequent probing and imaging was performed as described above for immunoblotting. Loading was visualised using Coomassie Blue on a duplicate gel.

In-gel assays were performed for complex I and II activity as described in²⁰.

### Digital Droplet PCR

### Mt-Nd5 Primers

Forward: 5′-TGCCTAGTAATCGGAAGCCTCGC-3′
Reverse: 5′-TCAGGCGTTGGTGTTGCAGG-3′

### VDAC1 Primers

Forward: 5′-CTCCCACATACGCCGATCTT-3′
Reverse: 5′-GCCGTAGCCCTTGGTGAAG-3′

Samples were prepared in triplicate in a 96-well plate using 1 ng of DNA, 100 nM of each primer, 10 μL of QX200 ddPCR EvaGreen Supermix and water to 20 μL. Droplet generation, PCR and measurements were then performed on the QX200 Droplet Digital PCR System (Bio-Rad Laboratories) as per the manufacturer's instructions with the primer annealing temperature set at 60° C.

### Seahorse Assay

The Seahorse XF Cell Mito Stress Test (Agilent) was performed as per the manufacturer's instructions. Briefly, cells were plated into a Seahorse 96-well plate at 2×10⁴ cells/well a day prior to the assay. A sensor cartridge was also allowed to hydrate in water at 37° C. overnight. The water was replaced with Seahorse XF Calibrant and the sensor cartridge was re-incubated for 45 mins. Oligomycin, FCCP, Rotenone and Antimycin A were then added to their respective seahorse ports to a final concentration of 1 μM in the well before sensor calibration on the Seahorse XFe96 Analyser (Agilent). Meanwhile, cell media was replaced with 150 μL Seahorse XF Media supplemented with 1% FBS, 25 mM glucose, 1 mM sodium pyruvate and 2 mM glutamine and incubated at 37° C. for 30 mins. The cell plate was then inserted into the analyser post-calibration and run.

For read normalisation, protein extraction and measurement was performed as described above.

### In Vitro Metabolomics

Cells were seeded two days prior to metabolite extraction to achieve 70-80% confluency on the day of extraction. Plates were incubated at 37° C. and 5% CO₂ overnight. The following day, cells were replenished with excess fresh media to prevent starvation at the point of extraction. For steady-state experiments, media was prepared as described above with the substitution of GLUTAMAX™ with 2 mM L-glutamine. For U-¹³C-glucose and 4-²H₁-glucose isotope tracing experiments, media was prepared as follows: DMEM, no glucose (Life Technologies) supplemented with 0.11 g/L sodium pyruvate, 2 mM L-glutamine, 20% FBS, 100 μg/mL uridine and 25 mM glucose isotope (Cambridge Isotopes). For isotope tracing experiments using U-¹³C-glutamine and 1-¹³C-glutamine, DMEM containing 4.5 g/L D-glucose and 0.11 g/L sodium pyruvate was supplemented with 20% FBS, 100 μg/mL uridine and 4 mM glutamine isotope (Cambridge Isotopes).

On the day of extraction, 20 μL of media was added to 980 μL of extraction buffer from each well. Cells were then washed twice with ice-cold PBS. Extraction buffer (50:30:20, v/v/v, methanol/acetonitrile/water) was then added to each well (600 μL per 2×10⁶) and incubated for 5 min at 4° C. Samples were centrifuged at 16,000 g for 10 mins at 4° C. and the supernatant was transferred to liquid chromatography-mass spectrometry (LC-MS) glass vials and stored at −80° C. until run on the mass spectrometer.

Mass spectrometry and subsequent targeted metabolomics analysis was performed as described in²¹. Compound peak areas were normalised using the total measured protein per well quantified with a modified Lzowry assay²¹.

### In Vitro Measurements of Fumarate

Samples were Prepared as Described Above.

Fumarate analysis was carried out using a Q Exactive Orbitrap mass spectrometer (Thermo Scientific) coupled to an Ultimate 3000 HPLC system (Themo Fisher Scientific). Metabolite separation was done using a HILIC-Z column (InfinityLab Poroshell 120, 150×2.1 mm, 2.7 μm, Agilent) with a mobile phase consisting of a mixture of A (40 mM ammonium formate pH=3) and B (90% ACN/10% 40 mM ammonium formate). The flow rate was set to 200 μL/min and the injection volume was 5 μL. The gradient started at 10% A for 2 min, followed by a linear increase to 90% A for 15 min; 90% A was then kept for 2 minutes, followed by a linear decrease to 10% A for 2 min and a final re-equilibration step with 10% A for 5 min. The total run time was 25 min. The Q Exactive mass spectrometer was operated in negative mode with a resolution of 70,000 at 200 m/z across a range of 100 to 150 m/z (automatic gain control (AGC) target of 1×10⁶ and maximum injection time (IT) of 250 ms).

siRNA Knockdown for Metabolomics

1.2×12⁴ cells were plated into 12-well cell culture plates and incubated at 37° C. and 5% CO₂ overnight. The following day, cells were transfected with 5 μL of 5 μM siRNA with 5 μL of DharmaFECT 1 Transfection Reagent (Horizon Discovery). Cells were either transfected with ON-TARGETplus MDH1 siRNA (L-051206-01-0005, Horizon Discovery) or ON-TARGETplus non-targeting control siRNA (D-001810-10-05, Horizon Discovery). Cells were supplemented with excess media the following day and metabolites extracted 48 hrs post-transfection as outlined above.

### LbNOX Treatment for Metabolomics

pUC57-LbNOX (addgene #75285) and pUC57-mitoLbNOX (addgene #74448) were gifted. Both enzyme sequences were amplified using Phusion PCR (Life Technologies) as per the manufacturer's instructions. These products were cloned into pcDNA3.1(−)_mCherry¹⁹ via the NheI and BamHI restriction sites and used for subsequent experiments.

Forward for LbNOX:
5′-GGTGGTGCTAGCCGCATGAAGGTCACCG-3′
Forward for mitoLbNOX:
5′-GGTGGTGCTAGCCGCATGCTCGCTACAAG-3′
Reverse:
5′-GGTGGTGGATCCTTACTTGTCATCGTCATC-3′

Cells were transfected and sorted as described above and 3×10⁴ mCherry+ cells were plated per well into a 12-well plate. Cells were allowed to recover overnight at 37° C. and 5% CO₂ followed by the addition of excess media to each well. Metabolites were extracted the following day and analysed as outlined above.

### Bulk Tumour Metabolomics

Tumour fragments (20-40 mg) were flash frozen on dry ice when harvested. Metabolites were extracted using the Precellys Evolution homogenizer (Bertin) with 25 μL of extraction buffer per mg of tissue. Samples were then centrifuged at 16,000 g for 10 mins at 4° C. and the supernatant was transferred to LC-MS glass vials and stored at −80° C. until analysis.

Samples were run and subsequent targeted metabolomics analysis was performed as described in²¹ Compound peak areas were normalised using the mass of the tissue.

### Calculating Cell Sensitivity to 2-DG

Cells were plated in a 96-well plate at 500 cells/well in 200 μL of cell culture media. Plates were incubated overnight at 37° C. and 5% CO₂. The following day, media was replaced with 0-100 mM 2-DG in quadruplicate. Plates were imaged once every 4 hrs on the IncuCyte Zoom (Essen Bioscience) for 5 days. Final confluency measurements were calculated using the system algorithm and the IC₅₀ was determined by GraphPad Prism.

### Bulk Tumour RNAseq

Tumour fragments (20-40 mg) were stored in RNAlater (Sigma) and stored at −80° C. Samples were sent to GeneWiz Technologies for RNA extraction and sequencing.

### HcMel12 Transduction

cytoLbNOX was cloned into the lentiviral plasmid pLex303 via the NheI and BamHI restriction sites and transduction of HcMel12 was performed as described in²². Transduced cells were selected via supplementation of 8 μg/mL blasticidin, and single clones were selected out from the surviving bulk population. cytoLbNOX expression was confirmed using immunoblotting.

pLEX303 was a gift from David Bryant (Addgene plasmid #162032; http://n2t.net/addgene:162032; RRID:Addgene_162032).

### Hartwig Dataset Analysis

The Hartwig Medical Foundation (HMF) dataset included WGS data from tumor metastases normal-matched samples from 355 melanoma patients (skin primary tumor location), of whom 233 had additional RNA sequencing data of the tumor samples. mtDNA somatic mutations were called and annotated as previously described¹. In brief, variants called by both Mutect2 and samtools mpileup were retained and merged using vcf2maf, which embeds the Variant Effect Predictor (VEP) variant annotator. Variants within the repeat regions (chrM:302-315, chrM:513-525, and chrM:3105-3109) were filtered out. Next, variants were filtered out if the Variant Allele Fraction (VAF) was lower than 1% in the tumor samples and lower than 0.24% in the normal sample, as previously described (Yuan et al, 2020). Finally, somatic variants were kept when supported by at least one read in both the forward and the reverse orientations. Samples with >50% VAF mtDNA Complex I truncating (frameshift indels, translation start site and nonsense mutations) and missense mutations were classified as mutated and the rest as wild-type. Gene expression data was obtained from the output generated by the isofox pipeline, provided by HMF. Adjusted Transcript per Million (“adjTPM”) gene counts per sample were merged into a matrix. Gene expression and mutation data were used to perform differential expression analysis with DESeg2 in R using the DESeqDataSetFromMatrix function. Gene set enrichment analysis (GSEA) was performed with fGSEA in R with a minimum set size of 15 genes, a maximum of 500 genes and 20,000 permutations, against the mSigDB Hallmark gene set collection (v.7.5.1). Normalized Enrichment Score (NES) were ranked for significant upregulated and downregulated gene sets.

### Statistical Methods

No statistical test was used to determine sample sizes. Mice were randomly assigned to different experimental groups. Samples were blinded to machine operators (metabolomics, proteomics, RNAseq). Researchers were blinded to experimental groups for in vivo anti-PD1 experiments. Specific statistical tests used to determine significance, group sizes (n) and P values are provided in the figure legends. P values <0.05, <0.01 and <0.001 are represented as *, ** and *** respectively in figures. All statistical analysis was carried out using Prism (GraphPad) and Rstudio.

### Data and Code Availability Statement

All non-commercial plasmids used have been deposited with addgene (Gammage Lab). All metabolomic data, mtDNA sequencing, bulk and single cell RNAseq and proteomic data contained in this study are available in the supplementary information or via specified public repositories.

mtDNA sequencing

Cellular DNA was amplified to create two ~8 kbp overlapping mtDNA products using PrimeStar GXL DNA Polymerase (Takara Bio) as per the manufacturer's instructions.

### Primers

Forward 1:
5′-ACTGATATTACTATCCCTAGGAGG-3′
Reverse 1:
5′-TTTGAGTAGAACCCTGTTAGG-3′
Forward 2:
5′-GGCCTGATAATAGTGACGC-3′
Reverse 2:
5′-GGTTGGGTTTAGTTTTTGTTTGG-3′

Resulting amplicons were sequenced using Illumina Nextera kit (150 cycle, paired-end). To determine the percentage of non-target C mutations in mtDNA, we first identified all C/G nucleotides with adequate sequencing coverage (>1000×) in both the reference and experimental sample. Then, for each of the 4 experimental samples, we identified positions for which sequencing reads in the experimental sample corresponded to G>A/C>T mutations. We further filtered the resulting list of mutations to retain only those with a heteroplasmy over 2%, and removed mutations that were also present in control samples. Finally, the non-target percentage was calculated as the fraction of total possible C/G positions that were mutated.

### Sample Preparation for MS Analysis

Cells were lysed in a buffer containing 4% SDS in 100 mM Tris-HCl pH 7.5 and 55 mM iodoacetamide. Samples were then prepared as previously described in²³ with minor modifications. Alkylated proteins were digested first with Endoproteinase Lys-C(1:33 enzyme:lysate) for 1 hr, followed by an overnight digestion with trypsin (1:33 enzyme:lysate). Digested peptides from each experimental condition and a pool sample were differentially labelled using TMT16-plex reagent (Thermo Scientific) as per the manufacturer's instructions. Fully labelled samples were mixed in equal amount and desalted using 100 mg Sep Pak C18 reverse phase solid-phase extraction cartridges (Waters). TMT-labelled peptides were fractionated using high pH reverse phase chromatography on a C18 column (150×2.1 mm i.d.—Kinetex EVO (5 μm, 100 Å)) on a HPLC system (LC 1260 Infinity II, Agilent). A two-step gradient was applied, 1% to 28% B (80% acetonitrile) over 42 min, then from 28% to 46% B over 13 min to obtain a total of 21 fractions for MS analysis

### UHPLC-MS/MS Analysis

Peptides were separated by nanoscale C18 reverse-phase liquid chromatography using an EASY-nLC II 1200 (Thermo Scientific) coupled to an Orbitrap Fusion Lumos mass spectrometer (Thermo Scientific). Elution was carried out using a binary gradient with buffer A (water) and B (80% acetonitrile), both containing 0.1% formic acid. Samples were loaded with 6 μl of buffer A into a 50 cm fused silica emitter (New Objective) packed in-house with ReproSil-Pur C18-AQ, 1.9 μm resin (Dr Maisch GmbH). Packed emitter was kept at 50° C. by means of a column oven (Sonation) integrated into the nanoelectrospray ion source (Thermo Scientific). Peptides were eluted at a flow rate of 300 nl/min using different gradients optimised for three sets of fractions: 1-7, 8-15, and 16-21²³. Each fraction was acquired for a duration of 185 minutes. Eluting peptides were electrosprayed into the mass spectrometer using a nanoelectrospray ion source (Thermo Scientific). An Active Background Ion Reduction Device (ESI Source Solutions) was used to decrease air contaminants signal level. The Xcalibur software (Thermo Scientific) was used for data acquisition. A full scan over mass range of 350-1400 m/z was acquired at 60,000 resolution at 200 m/z, with a target value of 500,000 ions for a maximum injection time of 50 ms. Higher energy collisional dissociation fragmentation was performed on most intense ions during 3 sec cycle time, for a maximum injection time of 120 ms, or a target value of 100,000 ions. Peptide fragments were analysed in the Orbitrap at 50,000 resolution.

### Proteomics Data Analysis

The MS Raw data were processed with MaxQuant software²⁴ v.1.6.1.4 and searched with Andromeda search engine²⁵, querying SwissProt²⁶ Mus musculus (25,198 entries). First and main searches were performed with precursor mass tolerances of 20 ppm and 4.5 ppm, respectively, and MS/MS tolerance of 20 ppm. The minimum peptide length was set to six amino acids and specificity for trypsin cleavage was required, allowing up to two missed cleavage sites. MaxQuant was set to quantify on “Reporter ion MS2”, and TMT16plex was set as the Isobaric label. Interference between TMT channels was corrected by MaxQuant using the correction factors provided by the manufacturer. The “Filter by PIF” option was activated and a “Reporter ion tolerance” of 0.003 Da was used. Modification by iodoacetamide on cysteine residues (carbamidomethylation) was specified as variable, as well as methionine oxidation and N-terminal acetylation modifications. The peptide, protein, and site false discovery rate (FDR) was set to 1%. The MaxQuant output ProteinGroup.txt file was used for protein quantification analysis with Perseus software²⁷ version 1.6.13.0. The datasets were filtered to remove potential contaminant and reverse peptides that match the decoy database, and proteins only identified by site. Only proteins with at least one unique peptide and quantified in all replicates in at least one experimental group were used for analysis. Missing values were added separately for each column. The TMT corrected intensities of proteins were normalised first by the median of all intensities measured in each replicate, and then by using LIMMA plugin²⁸ in Perseus. Significantly regulated proteins between two groups were selected using a permutation-based Student's t-test with FDR set at 1%.

### Mitochondrial Membrane Potential and pH Gradient

Membrane potential and pH gradient were measured using multi-wavelength spectroscopy as described in²⁹⁻³⁰. Briefly, cultured cells were disassociated by gentle tapping and then spun down and resuspended at a density of 1×10⁷ cells/mL in FluroBrite supplemented with 2 mM glutamine in a temperature-controlled chamber. Changes in mitochondrial cytochrome oxidation states were then measured with multi-wavelength spectroscopy. The baseline oxidation state was measured by back-calculation using anoxia to fully reduce the cytochromes, and a combination of 4 μM FCCP and 1 μM rotenone to fully oxidize the cytochromes. The membrane potential was then calculated from the redox poise of the b-hemes of the bc₁ complex and the pH gradient measured from the turnover rate and redox span of the bc₁ complex using a model of turnover³⁰.

### Mitochondrial NADH Oxidation State

Changes in NAD(P)H fluorescence were measured simultaneously with mitochondrial membrane potential using 365 nm excitation. The resultant emission spectrum was then measured with multi-wavelength spectroscopy²⁹. The baseline oxidation state of the mitochondrial NADH pool was back calculated using anoxia to fully reduce, and 4 μM FCCP to fully oxidize the mitochondrial NADH pool, respectively, assuming the cytosolic NADH pool and NADPH pools did not change with these interventions and short time period.

### H&E Staining

Haematoxylin and Eosin (H&E) staining and slide scanning was performed as described in³¹.

scRNAseq Methodology

### 1-Preprocessing of Single-Cell RNA Transcriptomics Data, Batch Effect Correction, and Clustering

CellRanger (v.7.0.1) was used to map the reads in the FASTQ files to the mouse reference genome (GRCm39)³². Seurat (v.4.2.0) package in R (v.4.2.1) was used to handle the pre-processed gene counts matrix generated by cellRanger³³. As an initial quality control step, cells with fewer than 200 genes as well as genes expressed in less than 3 cells were filtered out. Cells with >5% mitochondrial counts, UMI counts >37000, and gene counts <500 were then filtered out. The filtered gene counts matrix (31647 genes and 127356 cells) was normalized using the NormalizeData function using the log(Normalization) method and scale.factor to 10000. The FindVariableFeatures function was used to identify 2000 highly variable genes for principal component analysis. The first 50 principal components were selected for downstream analysis. RunHarmony function from harmony package (v.0.1.0) with default parameters was used to correct batch effects³⁴. The RunUMAP function with the reduction from “harmony” was used to generate UMAPs for cluster analysis. FindClusters function was used with the resolution parameters set to 1.6.

### 2—Epithelial Score

Average gene expression from cytokeratins, Epcan, and Sfn were used to calculate epithelial score.

### 3—Single-Cell Copy Number Estimation

CopyKat (v.1.1.0) was used to estimate the copy number status of each cell¹⁴. Parameters were set as ngene.chr=5, win.size=25, KS.cut=0.1, genome=“mm10” and cells annotated as T cells or NK cells in the UMAP as diploid reference cells.

### 4—Identification of Differential Expressed Marker Genes

Top differentially expressed genes in each cluster were identified using the FindAllMarkers function in the Seurat R package. Parameters for expression difference were set to be at least 1.25 times of fold changes (log fc.threshold=1.25) and adjusted p-value <0.05 with gene expression detected in at least 10% of cells in each cluster (min.pct=0.1). The top 20 highly differentially expressed genes in each cluster ranked by average fold change were defined as marker genes.

### 5—Pathway Enrichment Analysis of Single-Cell Transcriptomics Data

For cells in each identified cluster in the UMAP, the wilcoxauc function from presto R package (version 1.0.0) was used to conduct wilcox rank-sum test to obtain the fold change and p-value for all genes between cells in the high heteroplasmy group for both mutations and control group³⁵. The genes were ranked in decreasing order according to the formula sign(log 2FC)*(−log₁₀(p-value)). This ranked gene list, and mouse hallmark pathways (mh.all.v2002.1.Mm.symbols.gmt) from the MSigDB database were used as inputs for gene set enrichment analysis using the fgsea function from fgsea R package (v.1.22.0) with parameters of eps=0, minSize=5, maxSize=500³⁶.

### REFERENCES

1. Gorelick, A. N. et al. Respiratory complex and tissue lineage drive recurrent mutations in tumour mtDNA. Nat. Metab. (2021) doi:10.1038/s42255-021-00378-8.
2. Hopkins, J. F. et al. Mitochondrial mutations drive prostate cancer aggression. Nat. Commun. (2017) doi:10.1038/s41467-017-00377-y.
3. Schöpf, B. et al. OXPHOS remodeling in high-grade prostate cancer involves mtDNA mutations and increased succinate oxidation. Nat. Commun. 11, (2020).
4. Mok, B. Y. et al. A bacterial cytidine deaminase toxin enables CRISPR-free mitochondrial base editing. Nature 583, 631-637 (2020).
5. Kim, M., Mahmood, M., Reznik, E. & Gammage, P. A. Mitochondrial DNA is a major source of driver mutations in cancer. Trends in Cancer 8, 1046-1059 (2022).
6. Gorman, G. S. et al. Mitochondrial diseases. Nat. Rev. Dis. Prim. (2016) doi:10.1038/nrdp.2016.80.
7. Yuan, Y. et al. Comprehensive molecular characterization of mitochondrial genomes in human cancers. Nat. Genet. 52, 342-352 (2020).
8. Guerrero-Castillo, S. et al. The Assembly Pathway of Mitochondrial Respiratory Chain Complex I. Cell Metab. (2017) doi:10.1016/j.cmet.2016.09.002.
9. Graf, L. H., Kaplan, P. & Silagi, S. Efficient DNA-mediated transfer of selectable genes and unselected sequences into differentiated and undifferentiated mouse melanoma clones. Somat. Cell Mol. Genet. (1984) doi:10.1007/BF01534903.
10. Titov, D. V. et al. Complementation of mitochondrial electron transport chain by manipulation of the NAD+/NADH ratio. Science (80-.). (2016) doi:10.1126/science.aad4017.
11. Gaude, E. et al. NADH Shuttling Couples Cytosolic Reductive Carboxylation of Glutamine with Glycolysis in Cells with Mitochondrial Dysfunction. Mol. Cell 69, 581-593.e7 (2018).
12. Wang, Y. et al. Saturation of the mitochondrial NADH shuttles drives aerobic glycolysis in proliferating cells. Mol. Cell 82, 3270-3283.e9 (2022).
13. Dong, J. et al. Single-cell RNA-seq analysis unveils a prevalent epithelial/mesenchymal hybrid state during mouse organogenesis. Genome Biol. (2018) doi:10.1186/s13059-018-1416-2.
14. Gao, R. et al. Delineating copy number and clonal substructure in human tumors from single-cell transcriptomes. Nat. Biotechnol. (2021) doi:10.1038/s41587-020-00795-2.
15. Coffelt, S. B., Wellenstein, M. D. & De Visser, K. E. Neutrophils in cancer: Neutral no more. Nat. Rev. Cancer 16, 431-446 (2016).
16. Bald, T. et al. Ultraviolet-radiation-induced inflammation promotes angiotropism and metastasis in melanoma. Nature 507, 109-113 (2014).
17. Riaz, N. et al. Tumor and Microenvironment Evolution during Immunotherapy with Nivolumab. Cell (2017) doi:10.1016/j.cell.2017.09.028.
18. DeBerardinis, R. J. & Chandel, N. S. We need to talk about the Warburg effect. Nat. Metab. 2, 127-129 (2020).
19. Gammage, P. A., Van Haute, L. & Minczuk, M. Engineered mtZFNs for manipulation of human mitochondrial DNA heteroplasmy. in Methods in Molecular Biology vol. 1351 145-162 (Humana Press Inc., 2016).
20. Fernandez-Vizarra, E. & Zeviani, M. Blue-Native Electrophoresis to Study the OXPHOS Complexes. in Methods in Molecular Biology (2021). doi:10.1007/978-1-0716-0834-0_20.
21. Villar, V. H. et al. Hepatic glutamine synthetase controls N 5-methylglutamine in homeostasis and cancer. Nat. Chem. Biol. (2022) doi:10.1038/s41589-022-01154-9.
22. Nacke, M. et al. An ARF GTPase module promoting invasion and metastasis through regulating phosphoinositide metabolism. Nat. Commun. (2021) doi:10.1038/s41467-021-21847-4.
23. Cao, X. et al. The mammalian cytosolic thioredoxin reductase pathway acts via a membrane protein to reduce ER-localised proteins. J. Cell Sci. 133, (2020).
24. Cox, J. & Mann, M. MaxQuant enables high peptide identification rates, individualized p.p.b.-range mass accuracies and proteome-wide protein quantification. Nat. Biotechnol. 26, (2008).
25. Cox, J. et al. Andromeda: A peptide search engine integrated into the MaxQuant environment. J. Proteome Res. 10, 1794-1805 (2011).
26. Apweiler, R. The Universal Protein Resource (UniProt) in 2010. Nucleic Acids Res. 38, (2010).
27. Tyanova, S. et al. The Perseus computational platform for comprehensive analysis of (prote)omics data. Nature Methods (2016) doi:10.1038/nmeth.3901.
28. Ritchie, M. E. et al. limma powers differential expression analyses for RNA-sequencing and microarray studies. Nucleic Acids Res. 43, e47 (2015).
29. Rocha, M. & Springett, R. Measuring the functionality of the mitochondrial pumping complexes with multi-wavelength spectroscopy. Biochim. Biophys. Acta-Bioenerg. 1860, 89-101 (2019).
30. Kim, N., Ripple, M. O. & Springett, R. Measurement of the mitochondrial membrane potential and pH gradient from the redox poise of the hemes of the bc 1 complex. Biophys. J. 102, 1194-1203 (2012).
31. Papalazarou, V., Drew, J., Juin, A., Spence, H. J. & Nixon, C. Collagen-VI expression is negatively mechanosensitive in pancreatic cancer cells and supports the metastatic niche. J. Cell Sci. 135, (2022).
32. Zheng, G. X. Y. et al. Massively parallel digital transcriptional profiling of single cells. Nat. Commun. 201781 8, 1-12 (2017).
33. Hao, Y. et al. Integrated analysis of multimodal single-cell data. Cell 184, 3573-3587.e29 (2021).
34. Korsunsky, I. et al. Fast, sensitive and accurate integration of single-cell data with Harmony. Nat. Methods 2019 1612 16, 1289-1296 (2019).
35. Korsunsky, I., Nathan, A., Millard, N. & Raychaudhuri, S. Presto scales Wilcoxon and auROC analyses to millions of observations. bioRxiv 653253 (2019).
36. Korotkevich, G. & Sukhov, V. Fast gene set enrichment analysis. bioRxiv 1-29 (2016).

### Example 2

### Influence of Tumour-Infiltrating Neutrophils on Response to Immune Checkpoint Inhibitors

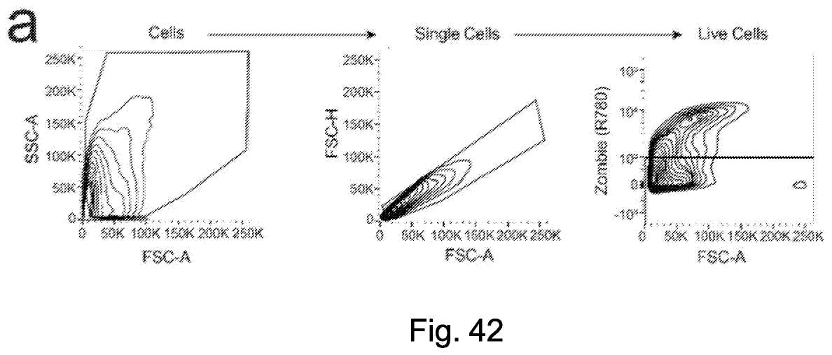

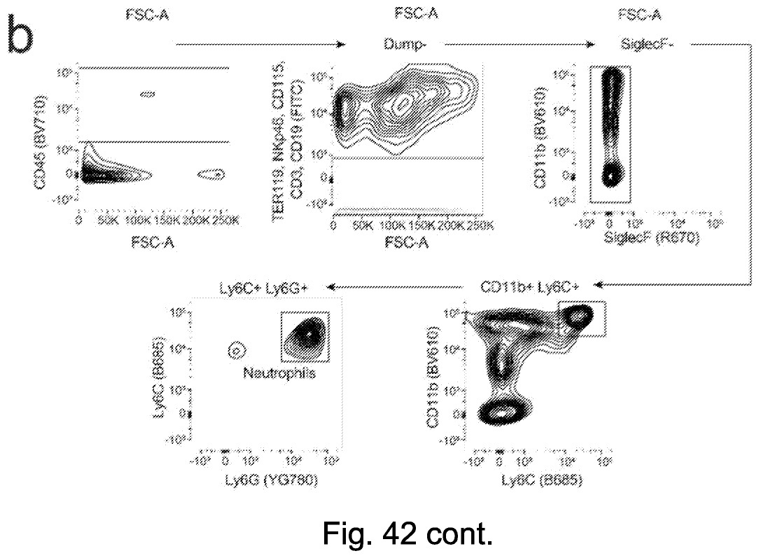

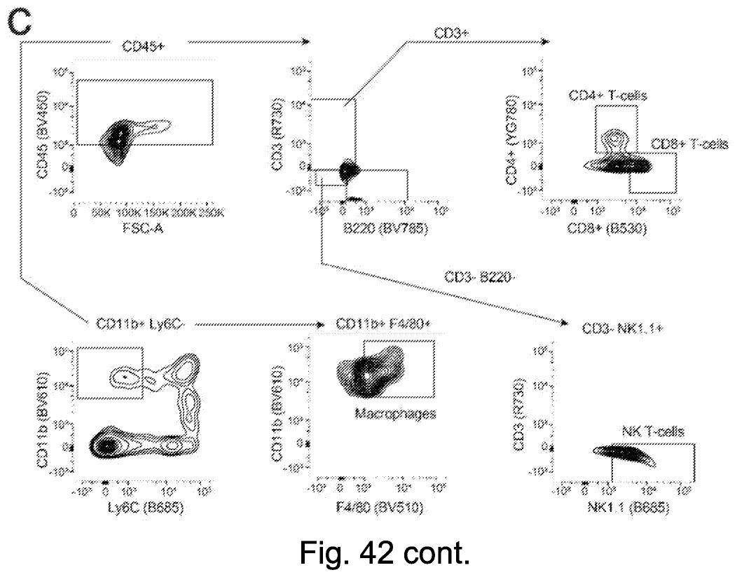

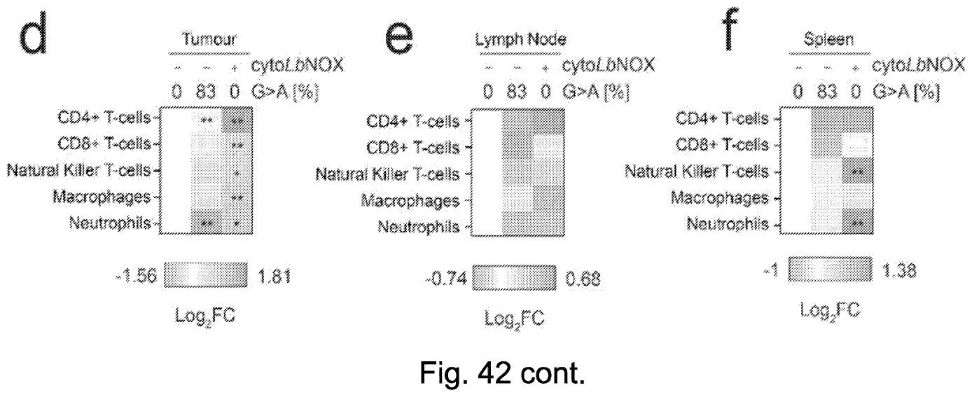

Tumour-associated neutrophils can function to suppress response to ICB in melanoma patients. Indeed, scRNAseq of B78-D14 mutant tumours revealed significant decreases in tumour neutrophil proportions (data not shown). This was further confirmed using flow cytometry (FIG. 42A-C), where the inventors observed decreased neutrophil infiltration in Hcmel12 m.12,43683% and cytoLbNOX tumours relative to wild-type (FIG. 42D). This was reflected in the tumour-draining lymph nodes (FIG. 42E). Interestingly, increases in CD4+ T-cells were observed in tumour and associated lymph nodes (FIG. 42D,E) whilst only increases in CD8+ T-cells, NK cells and macrophages were observed for cytoLbNOX tumours (FIG. 3.34D). Conversely, in the spleen, NK cells and neutrophils proportions were increased for mice allografted with mtDNA mutant and cytoLbNOX tumours compared to wild-type (FIG. 42F).

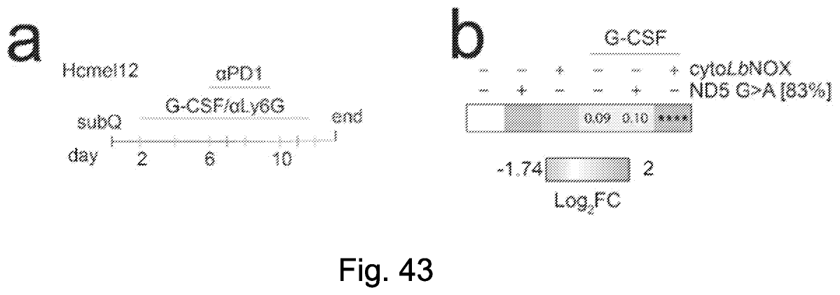

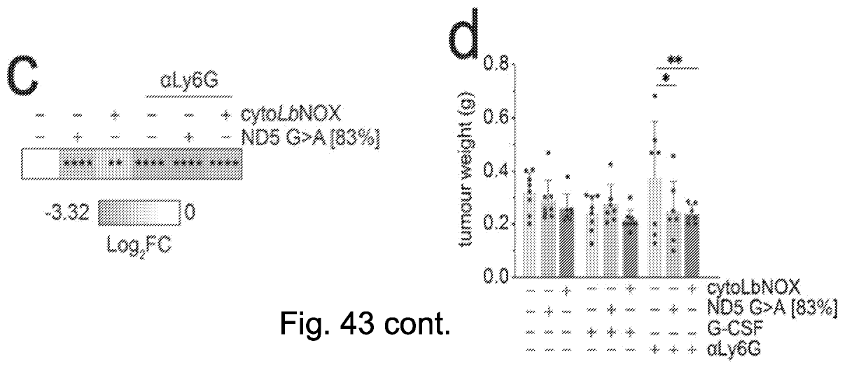

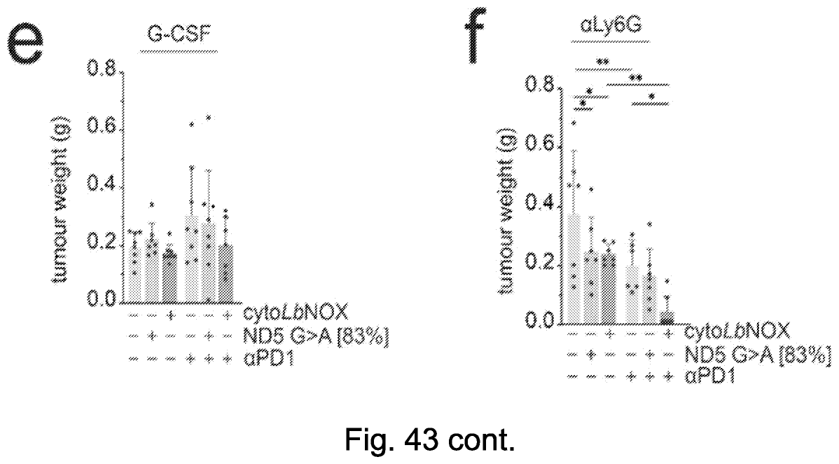

To determine if neutrophil depletions were necessary for observed sensitivity to ICB, the inventors chose to manipulate the proportion of tumour-associated neutrophils using G-CSF and anti-Ly6G treatments. Hcmel12 wild-type, m.12,436^{83%} and cytoLbNOX cells were allografted into C57BL/6 mice and were put on G-CSF or anti-Ly6G treatment with or without antiPD1 (FIG. 43A).

As expected, the inventors observed increases in tumour-associated neutrophils across genotypes using G-CSF whilst anti-Ly6G significantly reduced neutrophil proportions (FIG. 43 B,C). This did not affect tumour weight to untreated tumours when taken at the same end-point (FIG. 43D). Remarkably, G-CSF treatment abolished sensitivity of m.12,436^{83%} and cytoLbNOX tumours to anti-PD1 (FIG. 43E). Inversely, depletion of tumour-associated neutrophils rendered wild-type tumours sensitive to ICB treatment (FIG. 43F). Taken together, the inventors demonstrate that tumour-associated neutrophils coordinate and negatively regulate response to anti-PD1 therapy.

These results may justify the use of an agent that alters the lactate to glucose ratio (such as cytoLbNOX or another NADH oxidase) in combination with a tumour resident neutrophil depleting agent (such as anti-Ly6G antibody).

### Materials and Methods

### Use of Animal Models

Animal experiments were carried out in accordance with the UK Animals (Scientific Procedures) Act 1986 (P72BA642F) and by adhering to the ARRIVE guidelines with approval from the local Animal Welfare and Ethical Review Board of the University of Glasgow. Mice were housed in conventional cages in an animal room at a controlled temperature (19-23° C.) and humidity (55±10%) under a 12 hr light/dark cycle. Experiments only used male C57BL/6 mice at ~8 weeks of age which were injected subcutaneously with either 2.5×10⁵ B78 cells or 1×10⁴ HcMel12 cells, both prepared injected subcutaneously, prepared in PBS. Mice were culled at an endpoint of 15 mm tumour measurement.

For immunotherapy experiments, mice were put on a dosing regimen of 200 μg of anti-PD1 given intraperitoneally twice a week. The first dose was given 7 days post-injection and all mice were sacrificed at 21 or 13 days post-injection for B78 or HcMel12 cells respectively. Either 5 μg mouse recombinant G-CSF (Stemcell) or 100 μg anti-mouse Ly6G—clone 1A8 (2B Scientific) was given intraperitoneally every 2 days post-engraftment to mice for neutrophil depletion experiments.

### Example 3

### Response of Alternative Models of Melanoma to Immune Checkpoint Inhibitors

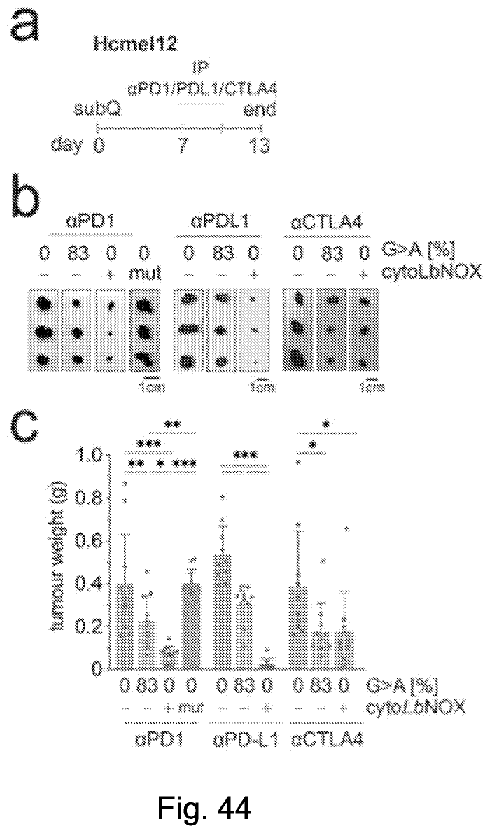

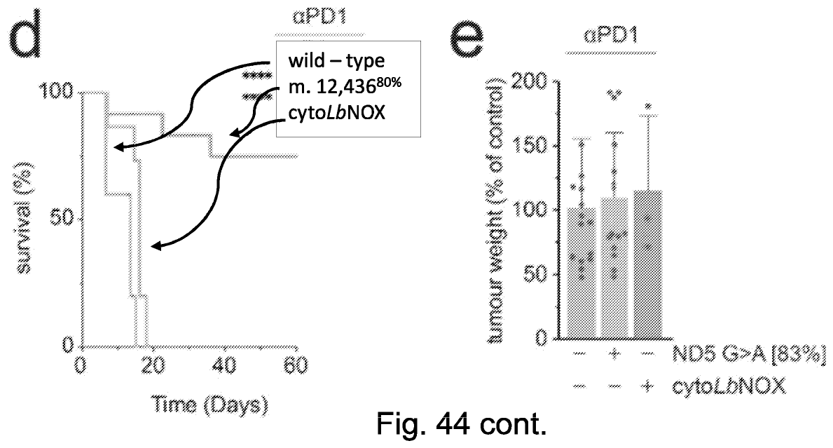

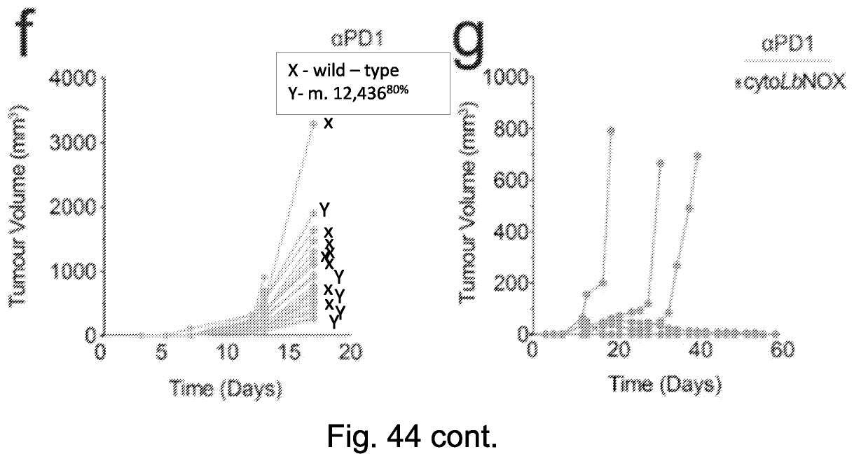

Remarkably, cytoLbNOX tumours were sensitive to anti-PD1 therapy whilst catalytic mutant tumours were not showing a role for redox dysfunction alone on immunotherapy. cytoLbNOX tumour weight was observed as <50% of mtDNA mutant tumours (FIG. 44A-C) which was reflected in anti-PDL1 treatments (FIG. 44A-C). Interestingly, anti-CTLA4 therapy, which regulated tumour growth through a spatially and temporally separate mechanism, lead to no differential reduction in tumour weight between mtDNA mutant and cytoLbNOX tumours (FIG. 44A-C). Further treatment of Hcmel12 wild-type, m.12436^{80%} and cytoLbNOX tumours with antiPD1 to an extended humane end-point demonstrated limited survival extension of mtDNA mutant tumour-bearing mice, whilst the majority of cytoLbNOX tumours demonstrated complete regression (FIG. 44D). Tumours that did reach end-points of 15 mm did not have differences in tumour weight or tumour volume (FIG. 44E-G).

Though both the immunogenic 4434 wild-type and m.12,436 tumours showed responses to anti-PD1 therapy, mtDNA mutant tumours completely regressed by day 20 whilst wildtype tumour weight remained measurable, maintaining our observations for differential response as in the non-immunogenic B78-D14 and Hcmel12 models (FIG. 45). Altogether, the data suggests that complex I truncations, irrespective of melanoma cell lineage, elicit a differential heteroplasmy dose-dependent response to immunotherapy, the mechanism for which may be mediated through alterations in redox balance, as observed through the use of cytoLbNOX-expressing Hcmel12 cells.

These results indicate that treatment with an agent that alters lactate to glucose ratio in a cancer or a pre-cancer may increase the sensitivity of already sensitive (at least to some extend) to immune checkpoint inhibitors cancers or pre-cancers.

### Example 4

### Sensitization of Contralateral WT Tumours to Checkpoint Inhibitors

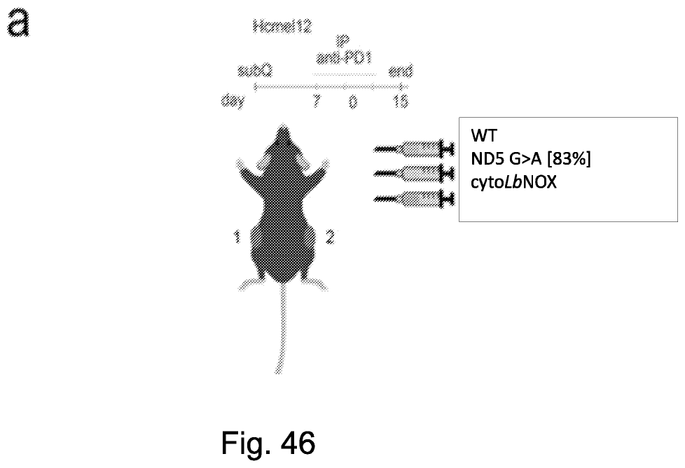

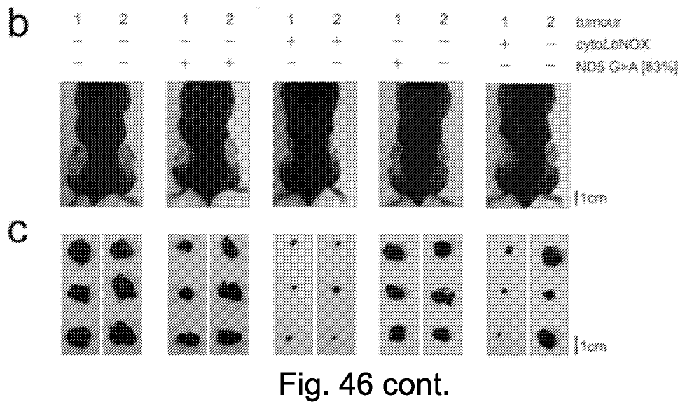

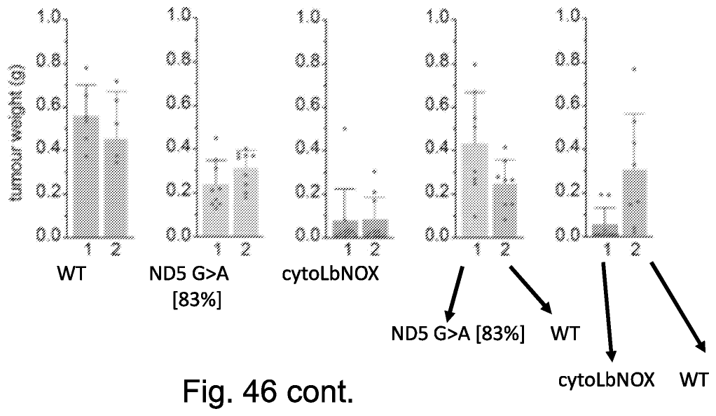

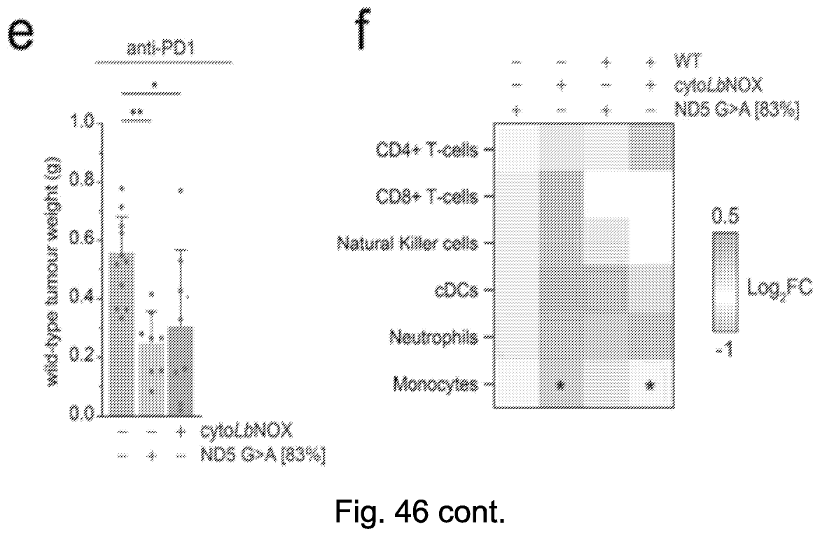

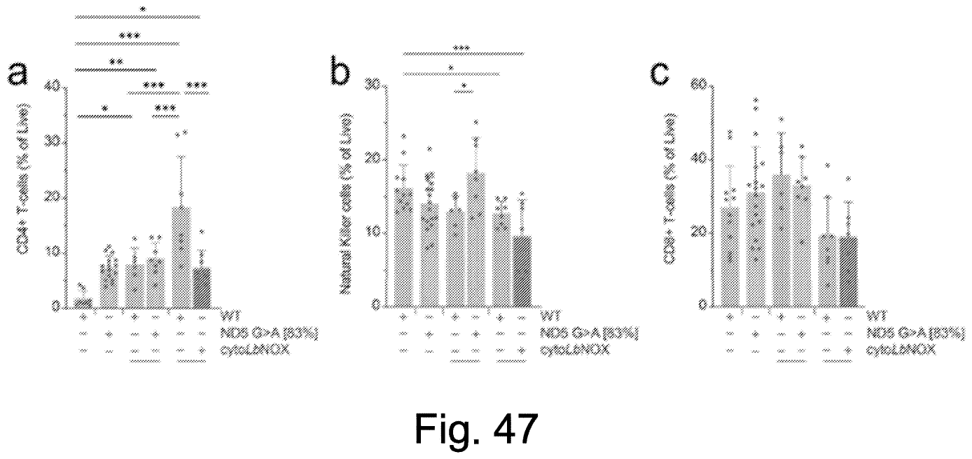

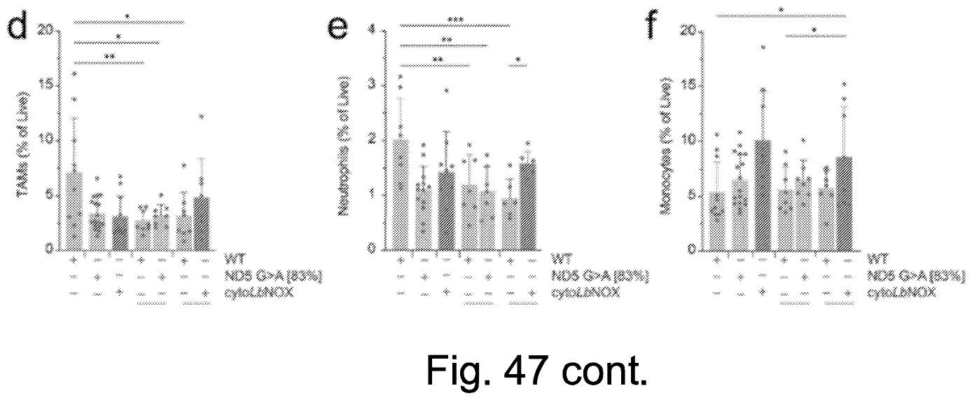

The inventors tested if the re-shaping of the immune environment extended beyond the tumour niche within their murine models of melanoma. Mice were subcutaneously injected on opposing flanks with Hcmel12 cells of either the same or different genotype and treated with anti-PD1 following the same regime as described previously (FIG. 46A). Mt.12,436^{83%} and cytoLbNOX tumours, when injected on each flank of the same mouse, responded to immunotherapy whilst wild-type tumours did not (FIG. 46B-D). Remarkably, however, wild-type tumours, when injected opposite to m.12,436^{83%} or cytoLbNOX tumours within the same mouse, were sensitised to anti-PD1 treatment (FIG. 46B-D). Comparison of wild-type tumour weights when injected opposite to m.12,436^{83%} or cytoLbNOX tumours reveal a ~50% decrease in tumour weight relative to wild-type tumours implanted opposite to wild-type (FIG. 46E). Interestingly, decreases in wildtype tumour weight showed no difference when injected opposite to either m.12,436^{83%} or cytoLbNOX suggesting both genotypes exert a similar effect on the systemic immune system (FIG. 46E).

Analysis of immune cell populations of circulating blood derived prior to end-point did not reveal any gross changes in cell proportions (FIG. 46F). However, further flow cytometry analysis of tumour immune populations revealed significant increases in CD4+ T-cells for m.12,436^{83%} and cytoLbNOX tumours as well as wild-type tumours when injected opposite to these genotypes (FIG. 3.47A). NK T-cells and CD8+ T-cells, conversely, revealed no significant changes in tumour-associated populations across samples (FIG. 46B,C). Due to the size of the cytoLbNOX tumours when injected on both the right and left flank, they could not be used for estimation of lymphoid cell populations.

Myeloid cell characterisation revealed decreases in TAMs and neutrophils and inverse increases in monocyte populations of responding tumours relative to non-responding wildtype, albeit not as pronounced as the changes observed for CD4+ T-cells (FIG. 46D-F).

Taken together, these data suggest the emergence of an abscopal effect following treatment with anti-PD1, indicating a systemic change in the immune environment as shown through parallel changes in immune cell proportions of wild-type tumours when injected opposite to m.12,436^{83%} and cytoLbNOX, but not wild-type.

### Example 5—mtDNA Mutations in Complex 4

Mt-Co1 is a mitochondrially encoded subunit of complex IV. The inventors made DddA-derived cytosine base editors (DdCBEs) to introduce a G>A point mutation in this protein at position m.6214 of the mouse mitochondrial genome.

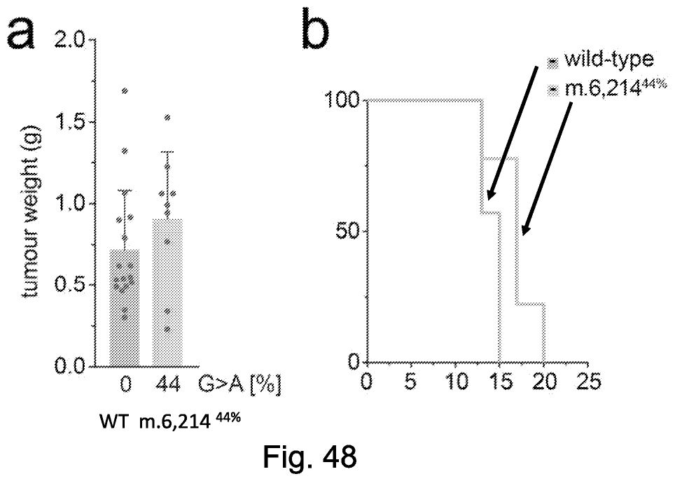

When implanted into B16 mice these tumours grew at comparable rates to wild-type, reaching comparable endpoint weight in similar time (FIG. 48A-B).

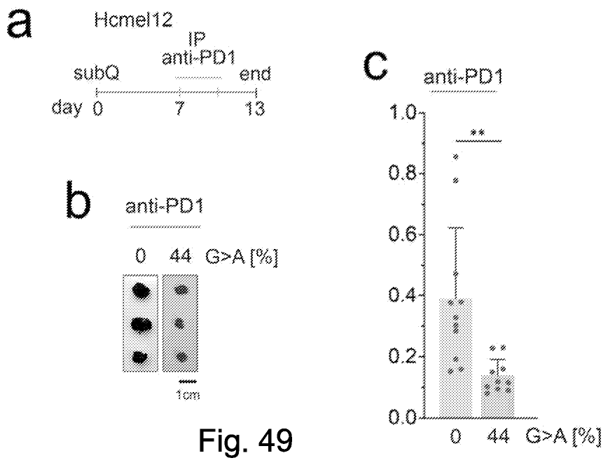

When challenged with anti-PD1, the Mt-Col mutant tumours showed a clear reduction in size at endpoint relative to wildtype tumours. This heteroplasmy is notably lower than that required for a robust immune response for Mt-Nd5 truncation—and this is likely due to the more profound effect on the respiratory chain that loss of complex IV will cause (FIG. 49A-C).

This results show that mutations in complexes other than complex I result in tumour sensitization to checkpoint inhibitors, further supporting that metabolic changes within the cells play an important role in increasing tumour sensitivity to checkpoint inhibitors.

### Example 6

### Increasing Sensitivity to Anti-CTLA4 Treatment and Anti-PD-L1 Treatment

As seen in FIG. 44B 12,436^{83%} tumors, and tumours that express cytoLbNOX were significantly smaller upon treatment with different types of immune checkpoint inhibitors (such as PD1 inhibitors, PD-L1 inhibitors, and CTLA4 inhibitors).

1.-2. (canceled)

3. A method of sensitising a subject having a cancer or a pre-cancer to an immune checkpoint inhibitor, comprising exposing the subject to an agent that alters the redox status in the cancer or pre-cancer.

4. A method of treating a cancer or a pre-cancer in a subject, comprising administering an immune checkpoint inhibitor to the subject, wherein the subject has been exposed to an agent that alters the redox status in the cancer or pre-cancer.

5. A method of treating a cancer or a pre-cancer in a subject, comprising:
(i) exposing the subject to an agent that alters the redox status in the cancer or pre-cancer; and
(ii) administering an immune checkpoint inhibitor to the subject.

6. The method according to claim 3, wherein the agent that alters the redox status alters the lactate to glucose ratio in the cancer or pre-cancer.

7. The method according to claim 6, wherein the agent increases the lactate to glucose ratio, optionally wherein the increase in lactate to glucose ratio is to above 3:1.

8. The method of claim 3, wherein a sample of the cancer or precancer has a deleterious mitochondrial DNA (mtDNA) mutation load of less than 50%.

9. The method of claim 8, wherein the deleterious mitochondrial DNA (mtDNA) mutation load is less than 40%, less than 30%, or less than 20%.

10. The method of claim 3, wherein the agent is selected from the group consisting of:
(a) a compound that drives glycolytic flux through MDH1, optionally wherein the compound is selected from the group consisting of: isocitrate, aconitate, citrate, oxaloacetate, NADH and NAD+ precursors;
(b) a compound that modulates NAD(H) redox handling via the malate-aspartate shuttle, optionally wherein the compound is selected from the group consisting of: isocitrate, aconitate, citrate, oxaloacetate, malate, fumarate, and argininosuccinate;
(c) lactate, pyruvate;
(d) a glucose metabolising enzyme and/or a lactate metabolising enzyme;
(e) an inhibitor of an enzyme that decreases glycolytic flux in cancer cells or pre-cancer cells, optionally wherein the enzyme is pyruvate dehydrogenase or pyruvate carboxylase, optionally wherein the inhibitor is a small molecule;
(f) an activator of an enzyme that increases glycolytic flux in cancer or pre-cancer cells;
(g) an activator of an enzyme that increases lactate efflux in cancer cells or pre-cancer cells, optionally wherein the enzyme is MDH1 or GAPDH, optionally wherein the activator is a small molecule;
(h) an inhibitor of an enzyme that decreases lactate efflux in cancer cells or pre-cancer cells;
(i) a small molecule inhibitor of an enzyme in the malate-aspartate shuttle, optionally wherein the enzyme is selected from the group consisting of GOT1, GOT2, MDH1, MDH2, Glutamate-Aspartate carrier, and a-ketoglutarate-malate carrier;
(j) a small molecule activator of an enzyme in the malate-aspartate shuttle, optionally wherein the enzyme is selected from the group consisting of GOT1, GOT2, MDH1, MDH2, Glutamate-Aspartate carrier, and a-ketoglutarate-malate carrier;
(k) an inhibitor of complex I, complex II, complex III or complex IV;
(l) a compound that increases a deleterious mtDNA mutation load in the cancer or pre-cancer, optionally wherein the compound induces a deleterious mtDNA mutation; and
(m) a compound that decreases neutrophils in the subject, and/or decreases neutrophils in the cancer or pre-cancer, optionally wherein the neutrophils are tumour infiltrating neutrophils (TANs).

11. The method of claim 3, wherein the agent is the enzyme NADH oxidase or a nucleic acid that encodes said enzyme, optionally wherein the enzyme is from Lactobacillus brevis, further optionally wherein the enzyme is selected from the group consisting of: cytoLbNOX and mitoLbNOX.

12. The method of claim 3, wherein the cancer or pre-cancer is selected from the group consisting of: a childhood cancer, haematological cancer, and a myeloid cancer.

13. The method of claim 12, wherein the childhood cancer is selected from the group consisting of: leukemia, brain cancer, spinal cord cancer, neuroblastoma, Wilms tumor, lymphoma, rhabdomyosarcoma, retinoblastoma, and bone cancer.

14. The method of claim 3, wherein the immune checkpoint inhibitor is selected from the group consisting of_a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and KIR inhibitor, optionally wherein the immune checkpoint inhibitor is selected from the group consisting of a PD-1 inhibitor, a PD-L1 inhibitor, and CTLA4 inhibitor, further optionally wherein the PD-1 inhibitor is nivolumab.

15. The method according to claim 10, wherein the compound that increases a deleterious mtDNA mutation load in the cancer or pre-cancer is selected from the group consisting of a mitochondrial base editing enzyme and a mitochondrial heteroplasmy manipulating enzyme.

16. The method of claim 8, wherein the deleterious mtDNA mutation is selected from the group consisting of:
(i) a tRNA mutation having a MitoTIP RAW score of at least 12.6, or at least 16.25;
(ii) a rRNA mutation;
(iii) a truncation mutation in a mtDNA gene;
(iv) a missense mutation in a mtDNA gene, wherein the missense mutation has an Apogee score of more than 0.5, optionally wherein the missense mutation is selected from a frameshift mutation, an insertion mutation or a deletion mutation; and
(v) a mutation in a mtDNA D-loop region selected from the group consisting of: the H-strand promoter (545-567), MT-HV2 (hypervariable segment 2) m.57-372, and MT-HV1 (hypervariable segment 1)—m.16024-16390.

17. The method of claim 16, wherein the deleterious mtDNA mutation is in a gene selected from the group consisting of: MT-ND5, MT-NDI, MT-ND2, MT-ND3, MT-ND4, MT-ND4L, MT-ND6, MT-CO1, MT-CO2, MT-CO3, MT-CYB, MT-ATP6, MT-ATP8, MT-TL1, MT-TA, MT-TC, MT-TD, MT-TE, MT-TF, MT-TG, MT-TH, MT-TI, MT-TK, MT-TL2, MT-TM, MT-TN, MT-TP, MT-TQ, MT-TR, MT-TS1, MT-TS2, MT-TT, MT-TV, MT-TW, MT-TY, MT-RNR1 and MT-RNR2.

18. The method of claim 17, wherein the MT-ND5 deleterious mtDNA mutation is a truncating mutation that is in a region selected from: m.12418-12425:A indel or m.12385-12390:C indel.

19. The method of claim 8, wherein the deleterious mtDNA mutation is a truncation, missense, insertion, or frameshift mutation.
